SEQUENCE LISTING (24 sequences)

SEQ ID NO: 1 - protein, 518 aa, Homo sapiens
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNCSGVQVGDNNYLTMQQTTALPTWGLAPSGKGRGLQHPPPVGSQEGPKDPEAWSRPQGWYNHSGK

SEQ ID NO: 2 - protein, 515 aa, Artificial sequence
  Synthetic
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNCSAAAADNNYLTMQQTTALPTWGPSGKGRGLQHPPPVGSQEGPKDPEAWSRPQGWYNHSGK

SEQ ID NO: 3 - protein, 671 aa, Artificial sequence
  Synthetic
MQPDMSLNVIKMKSSDFLESAELDSGGFGKVSLCFHRTQGLMIMKTVYKGPNCIEHNEALLEEAKMMNRLRHSRVVKLLGVIIEEGKYSLVMEYMEKGNLMHVLKAEMSTPLSVKGRIILEIIEGMCYLHGKGVIHKDLKPENILVDNDFHIKIADLGLASFKMWSKLNNEEHNELREVDGTAKKNGGTLYYMAPEHLNDVNAKPTEKSDVYSFAVVLWAIFANKEPYENAICEQQLIMCIKSGNRPDVDDITEYCPREIISLMKLCWEANPEARPTFPGIEEKFRPFYLSQLEESVEEDVKSLKKEYSNENAVVKRMQSLQLDCVAVPSSRSNSATEQPGSLHSSQGLGMGPVEESWFAPSLEHPQEENEPSLQSKLQDEANYHLYGSRMDRQTKQQPRQNVAYNREEERRRRVSHDPFAQQRPYENFQNTEGKGTAYSSAASHGNAVHQPSGLTSQPQVLYQNNGLYSSHGFGTRPLDPGTAGPRVWYRPIPSHMPSLHNIPVPETNYLGNTPTMPFSSLPPTDESIKYTIYNSTGIQIGAYNYMEIGGTSSSLLDSTNTNFKEEPAAKYQAIFDNTTSLTDKHLDPIRENLGKHWKNCARKLGFTQSQIDEIDHDYERDGLKEKVYQMLQKWVMREGIKGATVGKLAQALHQCSRIDLLSSLIYVSQN

SEQ ID NO: 4 - protein, 4 aa, Artificial sequence
  Synthetic
IQIG

SEQ ID NO: 5 - protein, 4 aa, Artificial sequence
  Synthetic
VQVG

SEQ ID NO: 6 - protein, 113 aa, Artificial sequence
  Synthetic
MASRGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLETS

SEQ ID NO: 7 - protein, 99 aa, Artificial sequence
  Synthetic
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKTS

SEQ ID NO: 8 - protein, 631 aa, Artificial sequence
  Synthetic
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNCSGVQVGDNNYLTMQQTTALPTWGLAPSGKGRGLQHPPPVGSQEGPKDPEAWSRPQGWYNHSGKVASRGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLETS

SEQ ID NO: 9 - protein, 671 aa, Artificial sequence
  Synthetic
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLETS

SEQ ID NO: 10 - protein, 784 aa, Artificial sequence
  Synthetic
MQPDMSLNVIKMKSSDFLESAELDSGGFGKVSLCFHRTQGLMIMKTVYKGPNCIEHNEALLEEAKMMNRLRHSRVVKLLGVIIEEGKYSLVMEYMEKGNLMHVLKAEMSTPLSVKGRIILEIIEGMCYLHGKGVIHKDLKPENILVDNDFHIKIADLGLASFKMWSKLNNEEHNELREVDGTAKKNGGTLYYMAPEHLNDVNAKPTEKSDVYSFAVVLWAIFANKEPYENAICEQQLIMCIKSGNRPDVDDITEYCPREIISLMKLCWEANPEARPTFPGIEEKFRPFYLSQLEESVEEDVKSLKKEYSNENAVVKRMQSLQLDCVAVPSSRSNSATEQPGSLHSSQGLGMGPVEESWFAPSLEHPQEENEPSLQSKLQDEANYHLYGSRMDRQTKQQPRQNVAYNREEERRRRVSHDPFAQQRPYENFQNTEGKGTAYSSAASHGNAVHQPSGLTSQPQVLYQNNGLYSSHGFGTRPLDPGTAGPRVWYRPIPSHMPSLHNIPVPETNYLGNTPTMPFSSLPPTDESIKYTIYNSTGIQIGAYNYMEIGGTSSSLLDSTNTNFKEEPAAKYQAIFDNTTSLTDKHLDPIRENLGKHWKNCARKLGFTQSQIDEIDHDYERDGLKEKVYQMLQKWVMREGIKGATVGKLAQALHQCSRIDLLSSLIYVSQNMASRGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLETS

SEQ ID NO: 11 - DNA, 1557 bp, Homo sapiens
atgtcgtgcgtcaagttatggcccagcggtgcccccgcccccttggtgtccatcgaggaactggagaaccaggagctcgtcggcaaaggcgggttcggcacagtgttccgggcgcaacataggaagtggggctacgatgtggcggtcaagatcgtaaactcgaaggcgatatccagggaggtcaaggccatggcaagtctggataacgaattcgtgctgcgcctagaaggggttatcgagaaggtgaactgggaccaagatcccaagccggctctggtgactaaattcatggagaacggctccttgtcggggctgctgcagtcccagtgccctcggccctggccgctcctttgccgcctgctgaaagaagtggtgcttgggatgttttacctgcacgaccagaacccggtgctcctgcaccgggacctcaagccatccaacgtcctgctggacccagagctgcacgtcaagctggcagattttggcctgtccacatttcagggaggctcacagtcagggacagggtccggggagccagggggcaccctgggctacttggccccagaactgtttgttaacgtaaaccggaaggcctccacagccagtgacgtctacagcttcgggatcctaatgtgggcagtgcttgctggaagagaagttgagttgccaaccgaaccatcactcgtgtacgaagcagtgtgcaacaggcagaaccggccttcattggctgagctgccccaagccgggcctgagactcccggcttagaaggactgaaggagctaatgcagctctgctggagcagtgagcccaaggacagaccctccttccaggaatgcctaccaaaaactgatgaagtcttccagatggtggagaacaatatgaatgctgctgtctccacggtaaaggatttcctgtctcagctcaggagcagcaataggagattttctatcccagagtcaggccaaggagggacagaaatggatggctttaggagaaccatagaaaaccagcactctcgtaatgatgtcatggtttctgagtggctaaacaaactgaatctagaggagcctcccagctctgttcctaaaaaatgcccgagccttaccaagaggagcagggcacaagaggagcaggttccacaagcctggacagcaggcacatcttcagattcgatggcccaacctccccagactccagagacctcaactttcagaaaccagatgcccagccctacctcaactggaacaccaagtcctggaccccgagggaatcagggggctgagagacaaggcatgaactggtcctgcaggaccccggagccaaatccagtaacagggcgaccgctcgttaacatatacaactgctctggggtgcaagttggagacaacaactacttgactatgcaacagacaactgccttgcccacatggggcttggcaccttcgggcaaggggaggggcttgcagcaccccccaccagtaggttcgcaagaaggccctaaagatcctgaagcctggagcaggccacagggttggtataatcatagcgggaaataa

SEQ ID NO: 12 - DNA, 1359 bp, Artificial sequence
  Synthetic
atgtcgtgcgtcaagttatggcccagcggtgcccccgcccccttggtgtccatcgaggaactggagaaccaggagctcgtcggcaaaggcgggttcggcacagtgttccgggcgcaacataggaagtggggctacgatgtggcggtcaagatcgtaaactcgaaggcgatatccagggaggtcaaggccatggcaagtctggataacgaattcgtgctgcgcctagaaggggttatcgagaaggtgaactgggaccaagatcccaagccggctctggtgactaaattcatggagaacggctccttgtcggggctgctgcagtcccagtgccctcggccctggccgctcctttgccgcctgctgaaagaagtggtgcttgggatgttttacctgcacgaccagaacccggtgctcctgcaccgggacctcaagccatccaacgtcctgctggacccagagctgcacgtcaagctggcagattttggcctgtccacatttcagggaggctcacagtcagggacagggtccggggagccagggggcaccctgggctacttggccccagaactgtttgttaacgtaaaccggaaggcctccacagccagtgacgtctacagcttcgggatcctaatgtgggcagtgcttgctggaagagaagttgagttgccaaccgaaccatcactcgtgtacgaagcagtgtgcaacaggcagaaccggccttcattggctgagctgccccaagccgggcctgagactcccggcttagaaggactgaaggagctaatgcagctctgctggagcagtgagcccaaggacagaccctccttccaggaatgcctaccaaaaactgatgaagtcttccagatggtggagaacaatatgaatgctgctgtctccacggtaaaggatttcctgtctcagctcaggagcagcaataggagattttctatcccagagtcaggccaaggagggacagaaatggatggctttaggagaaccatagaaaaccagcactctcgtaatgatgtcatggtttctgagtggctaaacaaactgaatctagaggagcctcccagctctgttcctaaaaaatgcccgagccttaccaagaggagcagggcacaagaggagcaggttccacaagcctggacagcaggcacatcttcagattcgatggcccaacctccccagactccagagacctcaactttcagaaaccagatgcccagccctacctcaactggaacaccaagtcctggaccccgagggaatcagggggctgagagacaaggcatgaactggtcctgcaggaccccggagccaaatccagtaacagggcgaccgctcgttaacatatac

SEQ ID NO: 13 - DNA, 2016 bp, Artificial sequence
  Synthetic
atgcaaccagacatgtccttgaatgtcattaagatgaaatccagtgacttcctggagagtgcagaactggacagcggaggctttgggaaggtgtctctgtgtttccacagaacccagggactcatgatcatgaaaacagtgtacaaggggcccaactgcattgagcacaacgaggccctcttggaggaggcgaagatgatgaacagactgagacacagccgggtggtgaagctcctgggcgtcatcatagaggaagggaagtactccctggtgatggagtacatggagaagggcaacctgatgcacgtgctgaaagccgagatgagtactccgctttctgtaaaaggaaggataattttggaaatcattgaaggaatgtgctacttacatggaaaaggcgtgatacacaaggacctgaagcctgaaaatatccttgttgataatgacttccacattaagatcgcagacctcggccttgcctcctttaagatgtggagcaaactgaataatgaagagcacaatgagctgagggaagtggacggcaccgctaagaagaatggcggcaccctctactacatggcgcccgagcacctgaatgacgtcaacgcaaagcccacagagaagtcggatgtgtacagctttgctgtagtactctgggcgatatttgcaaataaggagccatatgaaaatgctatctgtgagcagcagttgataatgtgcataaaatctgggaacaggccagatgtggatgacatcactgagtactgcccaagagaaattatcagtctcatgaagctctgctgggaagcgaatccggaagctcggccgacatttcctggcattgaagaaaaatttaggcctttttatttaagtcaattagaagaaagtgtagaagaggacgtgaagagtttaaagaaagagtattcaaacgaaaatgcagttgtgaagagaatgcagtctcttcaacttgattgtgtggcagtaccttcaagccggtcaaattcagccacagaacagcctggttcactgcacagttcccagggacttgggatgggtcctgtggaggagtcctggtttgctccttccctggagcacccacaagaagagaatgagcccagcctgcagagtaaactccaagacgaagccaactaccatctttatggcagccgcatggacaggcagacgaaacagcagcccagacagaatgtggcttacaacagagaggaggaaaggagacgcagggtctcccatgacccttttgcacagcaaagaccttacgagaattttcagaatacagagggaaaaggcactgcttattccagtgcagccagtcatggtaatgcagtgcaccagccctcagggctcaccagccaacctcaagtactgtatcagaacaatggattatatagctcacatggctttggaacaagaccactggatccaggaacagcaggtcccagagtttggtacaggccaattccaagtcatatgcctagtctgcataatatcccagtgcctgagaccaactatctaggaaatacacccaccatgccattcagctccttgccaccaacagatgaatctataaaatataccatatacaatagtactggcattcagattggagcctacaattatatggagattggtgggacgagttcatcactactagacagcacaaatacgaacttcaaagaagagccagctgctaagtaccaagctatctttgataataccactagtctgacggataaacacctggacccaatcagggaaaatctgggaaagcactggaaaaactgtgcccgtaaactgggcttcacacagtctcagattgatgaaattgaccatgactatgagcgagatggactgaaagaaaaggtttaccagatgctccaaaagtgggtgatgagggaaggcataaagggagccacggtggggaagctggcccaggcgctccaccagtgttccaggatcgaccttctgagcagcttgatttacgtcagccagaactaa

SEQ ID NO: 14 - DNA, 12 bp, Artificial sequence
  Synthetic
gtgcaagttgga

SEQ ID NO: 15 - DNA, 12 bp, Artificial sequence
  Synthetic
attcagattgga

SEQ ID NO: 16 - DNA, 339 bp, Artificial sequence
  Synthetic
atggcttctagaggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagt

SEQ ID NO: 17 - DNA, 324 bp, Artificial sequence
  Synthetic
atggcttctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagttatccgtacgacgtaccagactacgca

SEQ ID NO: 18 - DNA, 1893 bp, Artificial sequence
  Synthetic
atgtcgtgcgtcaagttatggcccagcggtgcccccgcccccttggtgtccatcgaggaactggagaaccaggagctcgtcggcaaaggcgggttcggcacagtgttccgggcgcaacataggaagtggggctacgatgtggcggtcaagatcgtaaactcgaaggcgatatccagggaggtcaaggccatggcaagtctggataacgaattcgtgctgcgcctagaaggggttatcgagaaggtgaactgggaccaagatcccaagccggctctggtgactaaattcatggagaacggctccttgtcggggctgctgcagtcccagtgccctcggccctggccgctcctttgccgcctgctgaaagaagtggtgcttgggatgttttacctgcacgaccagaacccggtgctcctgcaccgggacctcaagccatccaacgtcctgctggacccagagctgcacgtcaagctggcagattttggcctgtccacatttcagggaggctcacagtcagggacagggtccggggagccagggggcaccctgggctacttggccccagaactgtttgttaacgtaaaccggaaggcctccacagccagtgacgtctacagcttcgggatcctaatgtgggcagtgcttgctggaagagaagttgagttgccaaccgaaccatcactcgtgtacgaagcagtgtgcaacaggcagaaccggccttcattggctgagctgccccaagccgggcctgagactcccggcttagaaggactgaaggagctaatgcagctctgctggagcagtgagcccaaggacagaccctccttccaggaatgcctaccaaaaactgatgaagtcttccagatggtggagaacaatatgaatgctgctgtctccacggtaaaggatttcctgtctcagctcaggagcagcaataggagattttctatcccagagtcaggccaaggagggacagaaatggatggctttaggagaaccatagaaaaccagcactctcgtaatgatgtcatggtttctgagtggctaaacaaactgaatctagaggagcctcccagctctgttcctaaaaaatgcccgagccttaccaagaggagcagggcacaagaggagcaggttccacaagcctggacagcaggcacatcttcagattcgatggcccaacctccccagactccagagacctcaactttcagaaaccagatgcccagccctacctcaactggaacaccaagtcctggaccccgagggaatcagggggctgagagacaaggcatgaactggtcctgcaggaccccggagccaaatccagtaacagggcgaccgctcgttaacatatacaactgctctggggtgcaagttggagacaacaactacttgactatgcaacagacaactgccttgcccacatggggcttggcaccttcgggcaaggggaggggcttgcagcaccccccaccagtaggttcgcaagaaggccctaaagatcctgaagcctggagcaggccacagggttggtataatcatagcgggaaagtggcttctagaggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagt

SEQ ID NO: 19 - DNA, 2016 bp, Artificial sequence
  Synthetic
atgtcgtgcgtcaagttatggcccagcggtgcccccgcccccttggtgtccatcgaggaactggagaaccaggagctcgtcggcaaaggcgggttcggcacagtgttccgggcgcaacataggaagtggggctacgatgtggcggtcaagatcgtaaactcgaaggcgatatccagggaggtcaaggccatggcaagtctggataacgaattcgtgctgcgcctagaaggggttatcgagaaggtgaactgggaccaagatcccaagccggctctggtgactaaattcatggagaacggctccttgtcggggctgctgcagtcccagtgccctcggccctggccgctcctttgccgcctgctgaaagaagtggtgcttgggatgttttacctgcacgaccagaacccggtgctcctgcaccgggacctcaagccatccaacgtcctgctggacccagagctgcacgtcaagctggcagattttggcctgtccacatttcagggaggctcacagtcagggacagggtccggggagccagggggcaccctgggctacttggccccagaactgtttgttaacgtaaaccggaaggcctccacagccagtgacgtctacagcttcgggatcctaatgtgggcagtgcttgctggaagagaagttgagttgccaaccgaaccatcactcgtgtacgaagcagtgtgcaacaggcagaaccggccttcattggctgagctgccccaagccgggcctgagactcccggcttagaaggactgaaggagctaatgcagctctgctggagcagtgagcccaaggacagaccctccttccaggaatgcctaccaaaaactgatgaagtcttccagatggtggagaacaatatgaatgctgctgtctccacggtaaaggatttcctgtctcagctcaggagcagcaataggagattttctatcccagagtcaggccaaggagggacagaaatggatggctttaggagaaccatagaaaaccagcactctcgtaatgatgtcatggtttctgagtggctaaacaaactgaatctagaggagcctcccagctctgttcctaaaaaatgcccgagccttaccaagaggagcagggcacaagaggagcaggttccacaagcctggacagcaggcacatcttcagattcgatggcccaacctccccagactccagagacctcaactttcagaaaccagatgcccagccctacctcaactggaacaccaagtcctggaccccgagggaatcagggggctgagagacaaggcatgaactggtcctgcaggaccccggagccaaatccagtaacagggcgaccgctcgttaacatatacggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgagactagaggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagttaa

SEQ ID NO: 20 - DNA, 2355 bp, Artificial sequence
  Synthetic
atgcaaccagacatgtccttgaatgtcattaagatgaaatccagtgacttcctggagagtgcagaactggacagcggaggctttgggaaggtgtctctgtgtttccacagaacccagggactcatgatcatgaaaacagtgtacaaggggcccaactgcattgagcacaacgaggccctcttggaggaggcgaagatgatgaacagactgagacacagccgggtggtgaagctcctgggcgtcatcatagaggaagggaagtactccctggtgatggagtacatggagaagggcaacctgatgcacgtgctgaaagccgagatgagtactccgctttctgtaaaaggaaggataattttggaaatcattgaaggaatgtgctacttacatggaaaaggcgtgatacacaaggacctgaagcctgaaaatatccttgttgataatgacttccacattaagatcgcagacctcggccttgcctcctttaagatgtggagcaaactgaataatgaagagcacaatgagctgagggaagtggacggcaccgctaagaagaatggcggcaccctctactacatggcgcccgagcacctgaatgacgtcaacgcaaagcccacagagaagtcggatgtgtacagctttgctgtagtactctgggcgatatttgcaaataaggagccatatgaaaatgctatctgtgagcagcagttgataatgtgcataaaatctgggaacaggccagatgtggatgacatcactgagtactgcccaagagaaattatcagtctcatgaagctctgctgggaagcgaatccggaagctcggccgacatttcctggcattgaagaaaaatttaggcctttttatttaagtcaattagaagaaagtgtagaagaggacgtgaagagtttaaagaaagagtattcaaacgaaaatgcagttgtgaagagaatgcagtctcttcaacttgattgtgtggcagtaccttcaagccggtcaaattcagccacagaacagcctggttcactgcacagttcccagggacttgggatgggtcctgtggaggagtcctggtttgctccttccctggagcacccacaagaagagaatgagcccagcctgcagagtaaactccaagacgaagccaactaccatctttatggcagccgcatggacaggcagacgaaacagcagcccagacagaatgtggcttacaacagagaggaggaaaggagacgcagggtctcccatgacccttttgcacagcaaagaccttacgagaattttcagaatacagagggaaaaggcactgcttattccagtgcagccagtcatggtaatgcagtgcaccagccctcagggctcaccagccaacctcaagtactgtatcagaacaatggattatatagctcacatggctttggaacaagaccactggatccaggaacagcaggtcccagagtttggtacaggccaattccaagtcatatgcctagtctgcataatatcccagtgcctgagaccaactatctaggaaatacacccaccatgccattcagctccttgccaccaacagatgaatctataaaatataccatatacaatagtactggcattcagattggagcctacaattatatggagattggtgggacgagttcatcactactagacagcacaaatacgaacttcaaagaagagccagctgctaagtaccaagctatctttgataataccactagtctgacggataaacacctggacccaatcagggaaaatctgggaaagcactggaaaaactgtgcccgtaaactgggcttcacacagtctcagattgatgaaattgaccatgactatgagcgagatggactgaaagaaaaggtttaccagatgctccaaaagtgggtgatgagggaaggcataaagggagccacggtggggaagctggcccaggcgctccaccagtgttccaggatcgaccttctgagcagcttgatttacgtcagccagaacatggcttctagaggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagttaa

SEQ ID NO: 21 - protein, 222 aa, Homo sapiens [sequence omitted]

SEQ ID NO: 22 - DNA, 1231 bp, Artificial sequence
  Synthetic
ctagaattcagcggccgctttttttctagaattcagcgccgctgaattccacgcgggagggagagcagtgttctgctggagccgatgccaaaaaccatgcatttcttattcagattcattgttttcttttatctgtggggcctttttactgctcagagacaaaagaaagaggagagcaccgaagaagtgaaaatagaagttttgcatcgtccagaaaactgctctaagacaagcaagaagggagacctactaaatgcccattatgacggctacctggctaaagacggctcgaaattctactgcagccggacacaaaatgaaggccaccccaaatggtttgttcttggtgttgggcaagtcataaaaggcctagacattgctatgacagatatgtgccctggagaaaagcgaaaagtagttatacccccttcatttgcatacggaaaggaaggccatgcagaaggcaagattccaccggatgctacattgatttttgagattgaactttatgctgtgaccaaaggaccacggagcattgagacatttaaacaaatagacatggacaatgacaggcagctctctaaagccgagataaacctctacttgcaaagggaatttgaaaaagatgagaagccacgtgacaagtcatatcaggatgcagttttagaagatatttttaagaagaatgaccatgatggtgatggcttcatttctcccaaggaatacaatgtataccaacacgatgaactatagcatatttgtatttctactttttttttttagctatttactgtactttatgtataaaacaaagtcacttttctccaagttgtatttgctatttttcccctatgagaagatattttgatctccccaatacattgattttggtataataaatgtgaggctgttttgcaaacttaacttgcaggaatggtatcgactcgtgtttcctactgctttattctgtaaacaagaattgtagcaccatgaaacagacctctgggtcccagtgggcattttttcccctttcaggatgtaggaggacatgtatagtatgtcaaaaactgcaagcttttcccaactttaaccttaccagcatgttaatatccagtttttttatagtttaaaagttaaagtgcctcatattttgaaaatatccattaaggacccaggaattagcatttcacttgtttatacatttttataacattatgaagacgatataaaa

SEQ ID NO: 23 - protein, 456 aa, Artificial sequence
  Synthetic
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNCS

SEQ ID NO: 24 - protein, 628 aa, Artificial sequence
  Synthetic
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNCSAAAADNNYLTMQQTTALPTWGPSGKGRGLQHPPPVGSQEGPKDPEAWSRPQGWYNHSGKMASRGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLETS

(19) United States
(12) Patent Application Publication
(10) Pub. No.: US 2016/0160189 A1
(43) Pub. Date: Jun. 9, 2016

(54) Truncated Constructs of RIPK3 and Related Uses

(71) Applicant: ST. JUDE CHILDREN'S RESEARCH HOSPITAL, Memphis, TN (US)

(72) Inventors: Douglas Green, Germantown, TN (US); Andrew Oberst, Seattle, WA (US)

(73) Assignee: ST. JUDE CHILDREN'S RESEARCH HOSPITAL, Memphis, TN (US)

(21) Appl. No.: 14/888,336

(22) PCT Filed: Apr. 30, 2014
(86) PCT No.: PCT/US14/36196

§ 371 Date: Oct. 30, 2015

Related U.S. Application Data
(60) Provisional application No. 61/818,233, filed on May 1, 2013.

(51) Int. Cl. C12N 9/12; C07K 16/28; C07K 16/44

(52) U.S. Cl. CPC C12N 9/12; C07K 16/44; C07K 16/28; A61K 38/00

(57) ABSTRACT

The invention provides methods and compositions for inducing necroptosis in target cells, including for example cancer cells. This invention provides compositions and methods for the controlled expression and formation of full length RDPK3 homodimers, truncated RTPK3 oligomers and/or full length RIPK3/RIPK1 heterodimers in target cells both in vitro and in vivo therapeutic and research applications.

### FIELD OF INVENTION

The present invention relates generally to compositions and methods for inducing necroptosis in a target cell. In particular, necroptosis is induced using compositions including oligomers comprising RIPK3 proteins and RIPK1 proteins including, but not limited to, full length RIPK3 homodimers, truncated RIPK3 oligomers and/or full length RIPK3/RIPK1 heterodimers. More specifically, the present invention relates to methods of inducing necroptosis in a target cell, including for example the cells of a tumor, upon the controlled formation of such homodimers, oligomers and/or heterodimers in vitro and in vivo.

### BACKGROUND

Cell death is crucial for normal homeostasis and defects in this process underlie many human diseases. Necrotic cell death results from the interplay between several signaling pathways, and while the molecular mechanisms by which apoptosis and necrosis direct programmed cell death have been widely studied, including their implications for oncogenesis, the ability to direct cells towards a desired outcome remains elusive.

What is need is the ability to induce a cell (or cells), for example cancer cells or cells that will become cancerous, to proceed through the necrotic pathway.

### SUMMARY OF THE INVENTION

The present invention relates generally to compositions and methods for inducing necroptosis in a target cell. In particular, necroptosis is induced using compositions including oligomers comprising RIPK3 proteins and RIPK1 proteins including, but not limited to, full length RIPK3 homodimers, truncated RIPK3 oligomers and/or full length RIPK3/RIPK1 heterodimers. More specifically, the present invention relates to methods of inducing necroptosis in a target cell, including for example the cells of a tumor, upon the controlled formation of such homodimers, oligomers and/or heterodimers in vitro and in vivo. In one embodiment, the present invention contemplates an isolated protein comprising a truncated RIPK3. In one embodiment, the truncated RIPK3 lacks a RHIM domain. In one embodiment the truncated RIPK3 lacks a C-terminal domain. In one embodiment, the amino acid sequence of said truncated RIPK3 is SEQ ID NO: 2. In one embodiment, the amino acid sequence of said RHIM domain is selected from the group consisting of SEQ ID NO: 4 and SEQ ID NO: 5.

In one embodiment, the present invention contemplates, a fusion protein comprising a truncated RIPK3 and at least two binding proteins. In one embodiment, the truncated RIPK3 lacks a RHIM domain. In one embodiment, the amino acid sequence of said RHIM domain is selected from the group consisting of SEQ ID NO:4 and SEQ ID NO: 5. In one embodiment, the truncated RIPK3 lacks a C-terminal domain. In one embodiment, one of said at least two binding proteins comprises an Fv domain. In one embodiment, one said binding protein is selected from the group consisting of an FK506 binding protein and an FRB binding protein. In one embodiment, the amino acid sequence of the Fv domain is SEQ ID NO: 6. In one embodiment, the amino acid sequence of the FK506 binding protein is SEQ ID NO: 21. In one embodiment, the amino acid sequence of the FRB binding protein is SEQ ID NO: 7. In one embodiment, the fusion protein comprises RIPK3-Fv. In one embodiment, the amino acid sequence of the RIPK3-Fv fusion protein is SEQ ID NO: 8. In one embodiment, the fusion protein comprises RTPK3-2xFv. In one embodiment, the amino acid sequence of the RIPK3-2xFv fusion protein is SEQ ID NO: 9.

In one embodiment, the present invention contemplates an oligomer comprising at least one fusion protein comprising a truncated RIPK3 and at least two binding proteins. In one embodiment, at least three of said fusion proteins are attached at said binding proteins. In one embodiment, at least four of said fusion proteins are attached via said binding proteins. In one embodiment, at least five of said fusion proteins are attached via said binding proteins. In one embodiment, at least six fusion proteins are attached via said binding proteins. In one embodiment, the oligomer comprises a homodimer. In one embodiment, the oligomer comprises a heterodimer. In one embodiment, the heterodimer comprises a first fusion protein comprising a truncated RIPK3 and a first binding protein, and a second fusion protein comprising RIPK1 and a second binding protein. In one embodiment, the first binding protein is a FK506 binding protein and said second binding protein is a FRB binding protein. In one embodiment, the amino acid sequence of said truncated RIPK3 is SEQ ID NO:2 and the amino acid sequence of said RIPK1 is SEQ ID NO: 3. In one embodiment, the first fusion protein is attached to said second fusion protein via said first and second binding proteins. In one embodiment, the amino acid sequence of the FK506 binding protein is SEQ ID NO: 21. In one embodiment, the amino acid sequence of the FRB binding protein is SEQ ID NO:7. In one embodiment, the oligomer further comprises an RIPK1-Fv fusion protein. In one embodiment, the amino acid sequence of the RIPK1-Fv fusion protein is SEQ ID NO:10.

In one embodiment, the present invention contemplates a method of generating a truncated RIPK3 protein oligomer, comprising: a) providing: i) a fusion protein mixture, wherein said fusion proteins comprise a truncated RIPK3 protein and at least two binding proteins, and ii) a dimerizing agent, and b) adding said dimerizing agent to said fusion protein mixture under conditions such that a truncated RIPK3 protein oligomer is produced. In one embodiment, the truncated RIPK3 protein lacks an RHIM domain. In one embodiment, the amino acid sequence of said RHIM domain is selected from the group consisting of SEQ ID NO:4 and SEQ ID NO:5. In one embodiment, the amino acid sequence of said truncated RIPK3 protein lacks a C-terminal domain. In one embodiment, the oligomer comprises at least three of said fusion proteins. In one embodiment, the oligomer comprises at least four of said fusion proteins. In one embodiment, the oligomer comprises at least five of said fusion proteins. In one embodiment, the fusion proteins are attached via said binding proteins. In one embodiment, the dimerizing agent is rapamycin or a derivative thereof. In one embodiment, one of said at least two binding proteins is selected from the group consisting of an Fv domain, a FK506 binding protein and an FRB binding protein. In one embodiment, the amino acid sequence of the truncated RIPK3 protein is SEQ ID NO: 2. In one embodiment, the amino acid sequence of the Fv domain is SEQ ID NO: 6. In one embodiment, the amino acid sequence of the FK506 binding protein is SEQ ID NO: 21. In one embodiment, the amino acid sequence of the FRB binding protein is SEQ ID NO: 7. In one embodiment, the oligomer further comprises an RIPK1-Fv fusion protein. In one embodiment, the amino acid sequence of the RIPK1-Fv fusion protein is SEQ ID NO:10.

In one embodiment, the present invention contemplates a method of generating a truncated RIPK3 fusion protein comprising, a) providing: i) a first vector comprising a first nucleic acid sequence encoding a truncated RIPK3 protein, ii) a second vector comprising a second nucleic acid sequence encoding at least two binding proteins, iii) a dimerizing agent; and iv) a biological cell; wherein said first and second vectors are the same or different; b) introducing said first and second vectors into said biological cell; c) inducing expression of said truncated RIPK3 protein and said at least two binding proteins within said biological cell; and d) adding said dimerizing agent to said biological cells under conditions such that a truncated RIPK3 fusion protein comprising said truncated RIPK3 protein and said at least two binding proteins is generated. In one embodiment, the method further comprises step (e) creating a truncated RIPK3 oligomer wherein said truncated RIPK3 fusion proteins are attached by said at least two binding proteins. In one embodiment, the truncated RIPK3 oligomer comprises at least three of said truncated RIPK3 fusion proteins. In one embodiment, the truncated RIPK3 oligomer comprises at least four of said fusion proteins. In one embodiment, the truncated RIPK3 oligomer comprises at least five of said fusion proteins. In one embodiment, the oligomer comprises a heterodimer. In one embodiment, the oligomer comprises a homodimer. In one embodiment, one of said at least two binding domains is selected from the group consisting of an Fv domain, a FK506 binding protein and an FRB binding protein. In one embodiment, the amino acid sequence of said truncated RIPK3 protein comprises SEQ ID NO:2. In one embodiment, the dimerizing agent is rapamycin or a derivative thereof. In one embodiment, the nucleic acid sequence encoding the truncation RIPK3 protein is SEQ ID NO: 12. In one embodiment, the nucleic acid sequence encoding the Fv domain is SEQ ID NO: 16. In one embodiment, the nucleic acid sequence encoding the FK506 binding protein is SEQ ID NO: 22. In one embodiment, the nucleic acid encoding the FRB binding protein is SEQ ID NO: 17. In one embodiment, the first vector is selected from the group consisting of a pBabe-Puro retroviral vector and a pRRL lentiviral vector. In one embodiment, the second vector is selected from the group consisting of a pBabe-Puro retroviral vector and a pRRL lentiviral vector. In one embodiment, the oligomer further comprises an RIPK1-Fv fusion protein. In one embodiment, the amino acid sequence of the RIPK1-Fv fusion protein is SEQ ID NO:10.

In one embodiment, the present invention contemplates, a method of inducing necroptosis, comprising: a) providing: i) a biological cell, ii) a vector comprising a nucleic acid sequence encoding a fusion protein comprising a truncated RIPK3 and at least two binding proteins, and iii) a dimerizing agent; b) introducing said vector into said biological cell under conditions such that said fusion protein is expressed; and c) contacting said biological cell with said dimerizing agent under conditions such that said expressed fusion proteins form a truncated RIPK3 oligomer; d) inducing necroptosis in said biological cell with said truncated RIPK3 oligomer. In one embodiment, the truncated RIPK3 lacks a RHIM domain. In one embodiment, the truncated RIPK3 lacks a C-terminal domain. In one embodiment, the amino acid sequence of said truncated RIPK3 is SEQ ID NO: 2. In one embodiment, the amino acid sequence of said RHIM domain is selected from the group consisting of SEQ ID NO:4 and SEQ ID NO:5. In one embodiment, the truncated RIPK3 oligomer comprises a homodimer. In one embodiment, the truncated RIPK3 oligomer comprises a heterodimer. In one embodiment, one said at least two binding proteins are selected from the group consisting of an Fv domain, an FK506 binding protein and an FRB binding protein. In one embodiment, the dimerizing agent is rapamycin or a derivative thereof. In one embodiment, the biological cell is a tumor cell. In one embodiment, the tumor cell is derived from a patient diagnosed with cancer. In one embodiment, the introducing comprises administering said vector into said patient. In one embodiment, the administering is selected from the group consisting of an intravenous injection, an intramuscular injection, a subcutaneous injection and an intratumoral injection. In one embodiment, the contacting comprises administering said dimerizing agent to said patient. In one embodiment, the nucleic acid sequence encoding the truncation RIPK3 protein is SEQ ID NO: 12. In one embodiment, the nucleic acid sequence encoding the Fv domain is SEQ ID NO: 16. In one embodiment, the nucleic acid sequence encoding the FK506 binding protein is SEQ ID NO: 22. In one embodiment, the nucleic acid encoding the FRB binding protein is SEQ ID NO: 17. In one embodiment, the vector is selected from the group consisting of a pBabe-Puro retroviral vector and a pRRL lentiviral vector. In one embodiment, the oligomer further comprises an RIPK1-Fv fusion protein. In one embodiment, the amino acid sequence of the RTPK1-Fv fusion protein is SEQ ID NO:10.

In one embodiment, the present invention contemplates a method of treating cancer, comprising: a) providing: i) a cancerous cell within a patient, ii) a composition comprising a truncated RIPK3 oligomer comprising a fusion protein comprising a truncated RIPK3 and at least two binding proteins, and c) administering said composition to said patient; d) inducing necroptosis in said cancerous cell with said administered composition. In one embodiment, the truncated RIPK3 lacks a RHIM domain. In one embodiment, the truncated RIPK3 lacks a C-terminal domain. In one embodiment, the amino acid sequence of said truncated RIPK3 is SEQ ID NO: 2. In one embodiment, the amino acid sequence of said RHIM domain is selected from the group consisting of SEQ ID NO:4 and SEQ ID NO:5. In one embodiment, the truncated RIPK3 oligomer comprises a homodimer. In one embodiment, the truncated RIPK3 oligomer comprises a heterodimer. In one embodiment, one said at least two binding proteins are selected from the group consisting of an Fv domain, an FK506 binding protein and an FRB binding protein. In one embodiment, the composition comprises a liposome, a microbubble, a nanobubble, a microparticle and a nanoparticle. In one embodiment, the cancerous cell is a tumor cell. In one embodiment, the patient is diagnosed with cancer. In one embodiment, the administering is selected from the group consisting of an intravenous injection, an intramuscular injection, a subcutaneous injection and an intratumoral injection. In one embodiment, the amino acid sequence of the Fv domain is SEQ ID NO: 6. In one embodiment, the amino acid sequence of the FK506 binding protein is SEQ ID NO: 21. In one embodiment, the amino acid sequence of the FRB binding protein is SEQ ID NO: 7.

In one embodiment, the present invention contemplates a vector comprising a nucleic acid sequence encoding a fusion protein comprising a truncated RIPK3 and at least two binding proteins. In one embodiment, the truncated RIPK3 lacks a RHIM domain. In one embodiment, the truncated RIPK3 lacks a C-terminal domain. In one embodiment, the nucleic acid sequence of said RHIM domain is selected from the group consisting of SEQ ID NO: 14 and SEQ ID NO: 15. In one embodiment, one said at least two binding proteins are selected from the group consisting of an Fv domain, an FK506 binding protein and an FRB binding protein. In one embodiment, the nucleic acid sequence encoding the truncation RIPK3 protein is SEQ ID NO: 12. In one embodiment, the nucleic acid sequence encoding the Fv domain is SEQ ID NO: 16. In one embodiment, the nucleic acid sequence encoding the FK506 binding protein is SEQ ID NO: 22. In one embodiment, the nucleic acid encoding the FRB binding protein is SEQ ID NO: 17. In one embodiment, the vector is selected from the group consisting of a pBabe-Puro retroviral vector and a pRRL lentiviral vector.

In one embodiment, the present invention contemplates a mammalian cell comprising: a) a chimeric antigen receptor; b) a necrosis antigen having affinity for said chimeric antigen receptor; c) a suicide gene comprising a first nucleic acid sequence encoding a truncated RIPK3 protein. In one embodiment, the suicide gene further comprises a second nucleic acid sequence encoding an RIPK1 protein. In one embodiment, the suicide gene is inducible. In one embodiment, the truncated RIPK3 protein comprises an RIPK3^{ΔRHIM} protein. In one embodiment, the truncated RIPK3 protein comprises an. RIPK3^{ΔC} protein. In one embodiment, the truncated RIPK3 nucleic acid sequence is a truncated RIPK3 fusion protein. In one embodiment, the truncated RIPK3 fusion protein is an RIPK3^{ΔRHIM}-2xFv protein. In one embodiment, the truncated RIPK3 fusion protein is an RIPK3^{ΔC}-2xFv protein. In one embodiment, the necrosis antigen is encoded by a third nucleic acid. In one embodiment, the chimeric antigen receptor is encoded by a fourth nucleic acid. In one embodiment, the mammalian cell is stably transfected with said first, second, third and fourth nucleic acids.

In one embodiment, the present invention contemplates a method, comprising: a) providing; i) a mammalian cell comprising at least one chimeric antigen receptor and a suicide gene comprising a nucleic acid encoding a truncated RIPK3 protein; ii) a plurality of necrosis antigens; b) contacting at least one of the plurality of necrosis antigens with the at least one chimeric antigen receptor under conditions such that said suicide gene is expressed; and c) inducing necroptosis in the mammalian cell. In one embodiment, the suicide gene further comprises a second nucleic acid sequence encoding an RIPK1 protein. In one embodiment, the suicide gene is inducible. In one embodiment, the truncated RIPK3 protein comprises an RIPK3^{ΔRHIM} protein. In one embodiment, the truncated RIPK3 protein comprises an RIPK3^{ΔC} protein. In one embodiment, the truncated RIPK3 nucleic acid sequence is a truncated RIPK3 fusion protein. In one embodiment, the truncated RIPK3 fusion protein is an RIPK3^{ΔRHIM}-2xFv protein. In one embodiment, the truncated RIPK3 fusion protein is an RIPK3^{ΔC}-2xFv protein. In one embodiment, the necrosis antigen is encoded by a third nucleic acid. In one embodiment, the chimeric antigen receptor is encoded by a fourth nucleic acid. In one embodiment, the mammalian cell is stably transfected with said first, second, third and fourth nucleic acids.

In one embodiment, the present invention contemplates a method, comprising: a) providing; i) a tumor cell within a patient comprising a plurality of necrosis antigens; ii) a vector comprising a nucleic acid encoding at least one chimeric antigen receptor having affinity for at least one of the plurality of necrosis antigens and a suicide gene encoding a truncated RIPK3 protein; b) stably transfecting the tumor cell with said vector; and c) contacting the chimeric antigen receptor with the necrosis antigens under conditions such the said suicide gene is expressed; and d) inducing necroptosis in the tumor cell upon the expression of the suicide gene. In one embodiment, the suicide gene further encodes an RIPK1 protein. In one embodiment, the suicide gene is inducible. In one embodiment, the truncated RIPK3 protein comprises an RIPK3^{ΔRHIM} protein. In one embodiment, the truncated RIPK3 protein comprises an RIPK3^{ΔC} protein. In one embodiment, the truncated RIPK3 nucleic acid sequence is a truncated RIPK3 fusion protein. In one embodiment, the truncated RIPK3 fusion protein is an RIPK3^{ΔRHIM}-2xFv protein. In one embodiment, the truncated RIPK3 fusion protein is an RIPK3^{ΔC}-2xFv protein.

### DEFINITIONS

To facilitate the understanding of this invention a number of terms are defined below. Terms defined herein (unless otherwise specified) have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, terms defined in the singular are intended to include those terms defined in the plural and vice versa.

As used herein, absent an express indication to the contrary, the term “or” when used in the expression “A or B,” where A and B may refer to a composition, object, disease, product, etc., means one or the other (“exclusive OR”), or both (“inclusive OR”). As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the than “comprising” when placed before the recitation of steps in a method does not (although it may) require sequential performance of the listed steps, unless the context dictates otherwise. For example, a method comprising steps a, b, and c encompasses, for example, a method of performing steps in the order of steps a, c, and b, the order of steps c, b, and a, and the order of steps c, a, and b, etc.

Unless otherwise indicated, all numbers expressing quantities in the specification and claims are to be understood as being modified in all instances by the term “about”. Accordingly, unless indicated to the contrary, the numerical parameters in the specification and claims are approximations that may vary depending upon the desired properties sought to be obtained in a particular embodiment of the present invention. At the very least, and without limiting the application of the doctrine of equivalents to the scope of the claims, each numerical parameter should be construed in light of the number of reported significant digits and by applying ordinary rounding techniques. Any numerical value, however, inherently contains deviations that necessarily result from the errors found in the numerical value's testing measurements.

As used herein, the term “autophagy” refers to a survival response to cellular stress-associated damage or nutrient deprivation. Its primary function is to recycle proteins from engulfed cytoplasm or damaged organelles. Autophagy is recognized by the formation of autophagosomes, double membrane autophagic vacuoles that eventually fuse with lysosomes to form autolysosomes. Engulfed contents and the inner membrane of the autophagosome are subsequently degraded by lysosomal hydrolases. Various forms of environmental stress induce autophagy, which eventually results in either caspase-dependent or caspase-independent cell death.

As used herein, the term “necrosis” refers to a generally non-specific and unregulated destruction of tissues. Necrosis is generally caused by factors external to the cell or tissue, such as infection, toxins, or trauma and results in the release of intracellular contents.

As used herein, the term “apoptosis” refers to a form of programmed cell death in multicellular organisms orchestrated by a series of biochemical events that lead to a variety of morphological alterations, including blebbing, changes to the cell membrane (including loss of membrane asymmetry and attachment), cell shrinkage, nuclear fragmentation, chromatin condensation and chromosomal DNA fragmentation. Apoptosis is the major cell death pathway used to remove unwanted and harmful cells in a “clean or silent” manner during embryonic development, tissue homeostasis and immune regulation. An evolutionarily conserved family of cystein proteases, called caspases, is responsible for most of the observed morphological changes during apoptosis. Two distinct pathways initiate apoptosis: the extrinsic apoptotic pathway starting with the aggregation of death receptors, and the intrinsic apoptotic pathway starting with the release of mitochondrial factors in response to various stimuli, such as growth factor withdrawal, UV irradiation and cytotoxic drugs. Defective apoptotic processes have been implicated in an extensive variety of diseases; for example, defects in the apoptotic pathway have been implicated in diseases associated with uncontrolled cell proliferations, such as cancer.

As used herein, the term “necroptosis” refers to programmed cell death that is histologically similar to necrosis but distinct from apoptosis. For example, necroptosis characteristically involves cellular swelling and rupture, thereby releasing the intracellular contents. Unlike, apoptosis the cells do not shrink where the intracellular contents remain membrane-bound.

As used herein, the term “knockdown” refers to a method of selectively preventing the expression of a gene in a subject, patient or individual.

As used herein, the term “knockout”, “KO” or “knockout mice” refers to a genetically engineered mouse in which one or more genes (i.e. double and triple knockouts) have been inactivated via genetic manipulation. Knockout mice are important for studying the role of genes with a known sequence but unknown function. Knockout mice are widely used in investigating the genetics relating to human physiology.

As used herein, “Cre-Lox recombination” refers to a site-specific recombinase technology used to carry out in vivo site-specific recombination events, including deletions, insertions, translocations and inversions, in the genomic DNA. The cyclic recombinase (Cre) enzyme and the original Lox site called the LoxP sequence are derived from a bacteriophage P1. Cre-Lox recombination allows the DNA modification to be targeted to a specific cell type or be triggered by a specific external stimulus in both in eukaryotic and prokaryotic systems and is commonly used to circumvent embryonic lethality that often occurs following the systemic inactivation of one or more genes. The recombination event is mediated by Cre-recombinase, a site-specific enzyme that catalyzes the recombination of DNA between a pair of short target sequences called the LoxP sequences. These sequences contain specific binding sites for Cre that surround a directional core sequence where recombination can occur without inserting any extra supporting proteins or sequences. The result of the recombination event depends on the orientation of the loxP sites. For two lox sites on the same chromosome arm, inverted loxP sites will cause an inversion of the intervening DNA, while a direct repeat of loxP sites will cause a deletion event. If loxP sites are on different chromosomes it is possible for translocation events to be catalysed by Cre induced recombination. Placing Lox sequences appropriately will allow genes to be activated, repressed, or exchanged for other genes. The activity of the Cre enzyme can be controlled so that it is expressed in a particular cell type or triggered by an external stimulus, including for example, a chemical signal or a heat shock. In one embodiment, these targeted DNA changes are useful in cell lineage tracing and when mutants are lethal if expressed globally.

As used herein, the term “short interfering RNA” or “siRNA” refers to an intermediary molecule involved in triggering “RNA interference” (RNAi) in vertebrates and invertebrates and sequence-specific RNA degradation during posttranscriptional gene silencing in plants. In some embodiments siRNAs comprise a double-stranded (i.e. duplex) region of approximately 18-25 nucleotides and may contain approximately two to four unpaired nucleotides at the 3′ end of each strand. While not intending in any manner to limit the present invention to a particular mechanism, it is believed that at least one strand of the siRNA duplex region is substantially homologous (i.e. substantially complementary) to the target RNA molecule.

As used herein, the term “inflammatory response” refers to inflammation that occurs when tissues are injured by any number of causes, including for example, bacteria or virus infections, trauma, toxins and/or heat. Chemicals released by the damaged tissues (including cytokines, histamine, bradykinin and serotonin) cause blood vessels to leak fluid into the surrounding tissues resulting in local swelling. This helps isolate the foreign substance from further contact with body tissues. These chemicals also attract immune cells that function to clear microorganisms and dead or damaged cells by the process of phagocytosis.

As used herein, the term “cancer” relates to all forms of abnormal or improperly regulated reproduction of cells in a subject or patient. The growth and death of cancer cells is characteristically uncontrolled or inadequately controlled. Local accumulations of such cells may result in a “tumor”. A “malignant” tumor (as opposed to a “benign” tumor) comprises cells that migrate to nearby tissues, including cells that travel through the circulatory system to invade or colonize tissues or organs at considerable remove from their site of origin in the “primary tumor”. “Metastatic” cancer cells enter (“intravasate”) and exit (“extravasate”) blood vessel wells; tumors capable of releasing such cells are referred to as “metastatic.” For example, a metastatic breast cancer cell that has migrated to the lung is referred to as a “lung metastasis.” Metastatic cells may be identified herein by their respective sites of origin and destination, such as “breast-to-bone metastatic.” In the target tissue, a colony of metastatic cells may grow into a “secondary tumor”. Malignant tumors within the scope of the invention include, for example, carcinomas such as liver cancer, lung cancer, breast cancer, prostate cancer, cervical cancer, pancreatic cancer, colon cancer, ovarian cancer; stomach cancer, esophagus cancer, mouth cancer, tongue cancer, gum cancer, skin cancer (e.g., melanoma, basal cell carcinoma, Kaposi's sarcoma, etc.), muscle cancer, heart cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, and leukemia.

As used herein, the teens “patient” and “subject” refer to a human or animal who is ill or who is undergoing treatment for disease, but does not necessarily need to be hospitalized. For example, out-patients and persons in nursing homes are “patients”.

Agents that are useful in the invention's methods be administered to a subject by various routes including, for example, orally, intranasally, or parenterally, including intravenously, intramuscularly, subcutaneously, intraorbitally, intracapsularly, intrasynovially, intraperitoneally, intracisternally or by passive or facilitated absorption through the skin using, for example, a skin patch or transdermal iontophoresis. Furthermore, the agent can be administered by injection, intubation, via a suppository, orally or topically, the latter of which can be passive, for example, by direct application of an ointment or powder containing the agent, or active, for example, using a nasal spray or inhalant.

As used herein, the terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

In its broadest sense, the term “percent identical”, when used herein with respect to a nucleic acid molecule, means a nucleic acid molecule corresponding to a reference nucleotide sequence, wherein the corresponding nucleic acid molecule encodes a polypeptide having a substantially similar structure and function as the polypeptide encoded by the reference nucleotide sequence, e.g. where only changes in amino acids not affecting the polypeptide function occur. Desirably the substantially similar nucleic acid molecule encodes the polypeptide encoded by the reference nucleotide sequence. The term “substantially similar” is specifically intended to include nucleic acid molecules wherein the sequence has been modified to optimize expression in particular cells, e.g. in tumor cells. The percentage of identity between the substantially similar nucleic acid molecule and the reference nucleotide sequence desirably is at least 45%, more desirably at least 65%, more desirably at least 75%, preferably at least 85%, more preferably at least 90%, still more preferably at least 95%, yet still more preferably at least 99%. Preferably, the percentage of identity exists over a region of the sequences that is at least about 50 residues in length, more preferably over a region of at least about 100 residues, and most preferably the sequences are substantially similar over at least about 150 residues. In a most preferred embodiment, the sequences are substantially similar over the entire length of the coding regions. Sequence comparisons may be carried out using a Smith-Waterman sequence alignment algorithm and as described in more detail below (see e.g. Waterman, M. S. Introduction to Computational Biology: Maps, sequences and genomes. Chapman & Hall. London: 1995. ISBN 0-412-99391-0). The local S program, version 1.16, is used with following parameters: match: 1, mismatch penalty: 0.33, open-gap penalty: 2, extended-gap penalty: 2.

Another indication that a nucleic acid sequences is a substantially similar nucleic acid of the invention is that it hybridizes to a nucleic acid molecule of the invention under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0M Na ion, typically about 0.01 to 1.0M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially similar if the proteins that they encode are substantially similar. This occurs, e.g., when a copy of a nucleic acid is created using the maximum codon degeneracy permitted by the genetic code.

The term “percent identical”, when used herein with respect to a protein, means a protein corresponding to a reference protein, wherein the protein has a substantially similar structure and function as the reference protein, e.g. where only changes in amino acids sequence not affecting the polypeptide function occur. When used for a protein or an amino acid sequence the percentage of identity between the substantially similar and the reference protein or amino acid sequence desirably is at least 45% identity, more desirably at least 65%, more desirably at least 75%, preferably at least 85%, more preferably at least 90%, still more preferably at least 95%, yet still more preferably at least 99%, using default BLAST analysis parameters and as described in more detail below.

Optimal alignment of nucleic acid or protein sequences for comparison can be conducted as described above and, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

A further indication that two nucleic acid sequences or proteins are substantially similar is that the protein encoded by the first nucleic acid is immunologically cross reactive with, or specifically binds to, the protein encoded by the second nucleic acid. Thus, a protein is typically substantially similar to a second protein, for example, where the two proteins differ only by conservative substitutions.

As used herein, the term “nucleic acid” refers to a covalently linked sequence of nucleotides in which the 3′ position of the pentose of one nucleotide is joined by a phosphodiester group to the 5′ position of the pentose of the next, and in which the nucleotide residues (bases) are linked in specific sequence; i.e., a linear order of nucleotides. A “polynucleotide”, as used herein, is a nucleic acid containing a sequence that is greater than about 100 nucleotides in length. Nucleic acid molecules are said to have a “5′-terminus” (5′ end) and a “3′-terminus” (3′ end) because nucleic acid phosphodiester linkages occur to the 5′ carbon and 3′ carbon of the pentose ring of the substituent mononucleotides. The end of a nucleic acid at which a new linkage would be to a 5′ pentose carbon is its 5′ terminal nucleotide (by convention sequences are written, from right to left, in the 5′ to 3′ direction). The end of a nucleic acid at which a new linkage would be to a 3′ pentose carbon is its 3′ terminal nucleotide. A terminal nucleotide, as used herein, is the nucleotide at the end position of the 3′- or 5′-terminus DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring.

As used herein, the term “amino acid sequence” refers to an amino acid sequence of a protein molecule. “Amino acid sequence” and like terms, such as “polypeptide” or “protein”, are not meant to limit the amino acid sequence to the complete, native amino acid sequence associated with the recited protein molecule. Furthermore, an “amino acid sequence” can be deduced from the nucleic acid sequence encoding the protein.

### BRIEF DESCRIPTION OF THE DRAWINGS

The above and further advantages of the invention may be better understood by referring to the following description in conjunction with the accompanying drawings, in which:

FIG. 1 depicts a model of the regulation of cell death by the FADD-caspase-8-FLIP complex. In the presence of all three components, apoptosis does not proceed, and RIPK-dependent necrosis is blocked. In the absence of FADD or caspase-8, RIPK-dependent necrosis proceeds. If FLIP is absent, caspase-8-dependent apoptosis (as well as RIPK-dependent necrosis) proceed. If FLIP and RIPK3 are absent, only apoptosis proceeds (not shown). The model is consistent with our biochemical, cell based, and animal genetic experiments

FIG. 2 depicts the TNF-inducible complex of FADD, caspase-8, FLIP, RIPK1, and RIPK3. SVEC4-10 murine endothelial cells were treated with tumor necrosis factor (TNF) or zVAD as indicated for one hour. Cells were they lysed, and FADD was precipitated using the M19 anti-FADD antibody (Santa Cruz). Immune complexes, as well as whole cell lysate inputs, were resolved by western blotting using the indicated antibodies. * indicates the immunoglobluin light chain.

FIG. 3 shows exemplary data demonstrating that ablation of RIPK3 rescues development of FADD KO mice. Expected (Exp) and observed (Obs) frequencies of offspring of FADD^{+/-}RIPK3^{-/-}×FADD^{+/-}RIPK3^{-/-}.

FIG. 4 shows exemplary data demonstrating that RIPK3 ablation does not rescue e10.5 lethality in FLIP-null mice. Expected (Exp) and observed (Obs.) frequencies of embryos and offspring of FLIP^{+/-}RIPK3^{-/-}×FLIP^{+/-}RIPK3^{-/-}. * indicates clearly deformed but unabsorbed embryos (example on right).

FIG. 5 shows exemplary data demonstrating that FADD, FLIP, RIPK3 triple knockout (TKO) mice are viable. Expected (Exp) and observed (Obs.) frequencies of embryos and offspring of FLIP^{+/-}FADD^{-/-}RIPK3^{-/-}×FLIP^{+/-}FADD^{-/-}RIPK3^{-/-}.

FIG. 6 provides a schematic for inducible RIPK-necrosis. Two systems for inducible RIPK-necrosis. Fusion proteins RIPK1-FRB and RIPK3-FKBP are expressed in cells; heterodimerizer triggers death (FIG. 6A). RIPK3^{ΔC}-FKBPx2 is expressed in cells; homodimerizer triggers oligomerization of RIPK3 and death (FIG. 6B). Cell death in cells expressing RIPK1-FRB/RIPK3-FKBP and treated with heterodimerizer, and death was detected with annexin V-FITC. Death was inhibited by necrostatin-1 (not shown) (FIG. 6B). Cells expressing RIPK3ΔC-FKBPx2, treated with homodimerizer (1-100 nM); death was detected at 3 hrs by PI uptake. No effect of zVAD-fmk or necrostatin was observed (FIG. 6B).

FIG. 7 shows the results of an immunostaining of YFP in E10.5 embryos from endothelial/HSC reporter line. A pregnant LSL YFP reporter female×TIE2Cre transgenic male was perfused with formalin, and individual embryos were dissected. The embryos were embedded in paraffin and sectioned for staining with anti-YFP. YFP was observed in endothelium within the embryo and yolk sac (not shown) as well as in the endocardium (arrows). This may represent the developing endothelial/hematopoietic precursors. Control embryos lacking either TIE2Cre or LSL-YFP showed no staining with anti-YFP (not shown).

FIG. 8 depicts apoptosis in FLIP-RIPK3 double knockout (DKO) embryos. Mice were crossed to produce caspase-8 KO or FLIP-RIPK3 DKO embryos, taken at approx. e9.5. Genotyping was performed on yolk sac from individual embryos. Sections were stained with anti-cleaved caspase-3 (CM1) to detect cells undergoing apoptosis. While little staining was observed in the caspase-8 KO embryo, distinct regions of apoptosis were observed in the FLIP-RIPK3 DKO embryos (one of three is shown). Apoptosis was observed in endothelium as well as other tissues (red arrows) including the developing heart (upper arrow).

FIG. 9 provides a schematic depicting of the steps involved in generating the Venus-RIPK3 mouse. To generate the Venus-RIPK3 mouse, a targeting construct in which a LoxP-Frt-Neo-Frt cassette inserted in the 5′UTR of the RIPK3 gene (upstream of the CAP binding site), a second LoxP site inserted in intron 9 of the RIPK3 gene and a cDNA encoding the fluorescent protein Venus inserted in frame with the RIPK3 coding sequence will be used to replace the RIPK3 gene by homologous recombination in Embryomax (Millipore, CMTI-1) embryonic stem (ES) cells (129-svev background). Properly targeted ES cells will be injected into C57BL/6 blastocysts to produce chimeras Chimeras will next be crossed with C57BL/6 females to assess germ line transmission. The resulting allele (targeted allele) is inactive. To generate mice expressing Venus-RIPK3, mice bearing the targeted allele will be crossed with a FLP-deleter strain (B6(C3)-Tg(Pgk1-FLPo)10Sykr/J). The resulting allele (conditional allele) will express the Venus-RIPK3 fusion protein. To generate a null allele, mice bearing the conditional allele will be crossed with a CRE delete strain (B6.C-Tg(CMV-cre)1Cgn/J).

FIG. 10 provides a summary of the expression of proteins in caspase-8-deficient neuroblastoma (NB) lines. Summary of results from immunoblots with the indicated lines (NB1, NB2, etc; PCL-1691); actin serves as a control and (+/−) indicates detectable but low expression. CYLD appeared as a doublet in some lines, but only the higher form (potentially phosphorylated (81)) was observed in most. The “?” indicates that confirmation is required with phospho-CYLD-specific antibodies.

FIG. 11 provides a graph of tumor growth in Eμ-Myc+ animals treated with zVAD-fmk and anti-CD95 versus vehicle. EμMyc+p53^{ΔPP/wt} tumor-bearing animals were injected intravenously once/day for 5 days starting at P35 with 10 μg of the antagonist anti-CD95 Jot antibody and 200 μg of zVAD-fmk or vehicle control. Tumor size was monitored by ultrasound of the thymus, where volume was calculated from 3D reconstructions of the scanned tumors.

FIG. 12 presents exemplary data of RIPK3 dimerization seeding a RHIM-dependent necrosome complex. A) Schematic representation of the dimerizable and oligomerizable RIPK3 constructs used in this study. These constructs were cloned upstream of a T2A-GFP sequence, such that RIPK3 constructs contain both N-terminal FLAG and C-terminal 2A epitope tags. B&C) NIH-3T3 cells stably expressing RIPK3-1xFv were treated with indicated concentrations of AP1 (B), or with 30 nM AP1 in the presence or absence of 200 ng/ml TNFR1-Fc (C), and cell death was assessed over time using an IncuCyte imaging system. D) NIH-3T3 cells stably expressing RIPK3^{ΔRHIM}-1xFv were treated with increasing doses of dimerizer. E) NIH-3T3 cells stably expressing indicated constructs were treated as indicated, lysed, and necrosome complexes were covalently crosslinked using DSS. Resulting complexes were resolved by western blotting. Nec1 and zVAD were used at 30 μM and 50 μM, respectively, throughout.

FIG. 13 presents exemplary data showing: A) Jax cells, which express endogenous RIPK3, or NIH-3T3 cells, which do not, were treated with 10 ng/ml recombinant TNF along with inhibitors as indicated; B) Lysates from Jax cells, or NIH-3T3 cells stably expressing indicated constructs, were resolved by Western blot using the indicated antibodies. Note that the RIPK3 antibody used recognizes an epitope in the C-terminus, which is lacking in the RIPK3^{ΔC} construct; C&D) NIH-3T3 cells stably expressing RIPK3-1xFV were treated with recombinant TNF, 30 μM Nec1, 50 μM zVAD, or 200 ng/ml TNFR1-Fc as indicated. *p=0.0002; E-H) NIH-3T3 cells stably expressing catalytically inactive RIPK3^{K51A}-1xFV, phosphorylation site mutant RIPK3^{T231A,S232A}-1xFV, RHIM domain point mutant RIPK3^{ΔRHIM}-1xFV, or RHIM-truncated RIPK3^{ΔC}-1xFV were treated as indicated; I) NIH-3T3 cells stably expressing the indicated constructs were lysed and resolved by western blotting. Jax cells are included as a control for endogenous RIPK3 expression.

FIG. 14 presents exemplary data showing: A) NIH-3T3 cells were transfected with indicated siRNAs. Seventy-two hours later, lysates were collected and expression of indicated proteins was assessed by western blot; B) NIH-3T3 cells stably expressing RIPK3-1xFV were treated with 30 nM AP1, 50 μM zVAD and 200 ng/ml TNFR1-Fc as indicated; C) Densitometric analysis of the RIPK3 dimer and oligomer bands depicted in FIG. 2D; D) 313-NIH cells stably expressing RIPK3-1xFV were treated with AP1, then lysates were collected and subjected to DSS-mediated chemical crosslinking. These complexes were then resolved by western blotting using the indicated antibodies; E) RIPK1-associated immunocomplexes were purified as described in FIG. 1E, and co-precipitation of FADD was assessed by western blotting; F) NIH-3T3 cells stably expressing RIPK3^{ΔC}-2xFV were transfected with indicates siRNAs. Seventy-two hours later these cells were treated as indicated. All cell death measurements were performed using an IncuCyte bioimager as described.

FIG. 15 presents exemplary data that receptor-independent RIPK3 oligomerization is regulated by RIPK1 and caspase-8. A-C) NIH-3T3 cells stably expressing RIPK1-1xFv were treated with 30 nM AP1 and the indicated inhibitors, and cell death was assayed by IncuCyte. In B, cells were transfected with indicated siRNAs, then treated with AP1 72 hours later. D) NIH-3T3 cells stably expressing RIPK3-1xFv were treated with 30 nM AP1, as well as Nec1 or zVAD as indicated, and resulting complexes were resolved by western blotting. E) NIH-3T3 cells expressing the indicated constructs were treated as shown for 30 minutes, then lysed and subjected to immunoprecipitation using an antibody to the FLAG epitopes expressed on the RIPK3 constructs. Immune complexes were purified and resolved using TruBlot reagents to avoid aspecific signals from immunoglobulins, as described. Nec1 and zVAD were used at 30 μM and 50 μM, respectively, throughout.

FIG. 16 presents exemplary data of chemically-enforced RIPK3 oligomerization activates RIPK3 in the absence of the RHIM domain. A-D) NIH-3T3 cells expressing RIPK3-2xFv (A&C) or RIPK3^{ΔC}-2xFv (B&D) were treated as indicated, and cell death was assessed using an IncuCyte as described. E) NIH-3T3 cells stably expressing indicated constructs were treated as indicated, lysed, and necrosome complexes were covalently cross-linked using DSS. Resulting complexes were resolved by western blotting. F) NIH-3T3 cells expressing the indicated constructs were treated as shown for 30 minutes, then lysed and subjected to immunoprecipitation using an antibody to the FLAG epitopes expressed on the RIPK3 constructs. Immune complexes were purified and resolved using TruBlot reagents to avoid aspecific signals from immunoglobulins, as described. Nec1 and zVAD were used at 30 μM and 50 μM, respectively, throughout.

FIG. 17 presents exemplary data showing that RIPK1 inhibits spontaneous RIPK3 oligomerization and necroptosis. A) NIH-3T3 cells expressing RIPK3-1xFv were transfected with indicated siRNAs, then with 30 nM AP1 or 30 μM Nec1 72 h later. For scramble vs. RIPK1 siRNA treated with AP1, ****p<0.001, ***p=0.0056, **p=0.019, *p=0.037. B) NIH-3T3 cells stably expressing DD-RIPK3 were treated with 1 μM Shield drug for indicated times, then lysed and resolved by Western blot. GFP, which is translated from the same mRNA as DD-RIPK3 but is not destabilized, was also resolved. Jax cells expressing endogenous RIPK3 are included as a control. C, D & E) NIH-3T3 cells expressing DD-RIPK3 were transfected with indicated siRNAs, then treated 72 h later with 1 ng/ml recombinant TNF (C) or 1 μM Shield drug (D&E) and 30 μM Nec1 as indicated. F&G) Proposed model. F) Formation of a RIPK3 dimer via C-terminal dimerization is not sufficient to allow autophosphorylation and activation. Rather, dimerization seeds a RHIM-dependent oligomer that recruits additional molecules of RIPK3 to promote autophosphorylation, MLKL activation, and necroptosis. G) RIPK1 is recruited to the totaling RIPK3 oligomer via RHIM-RHIM interactions. This promotes recruitment of suppressive proteins including the caspase-8/FLIP complex. RIPK1 thereby exerts intrinsic suppression of RIPK3 oligomerization in the absence of receptor signals.

FIG. 18 presents exemplary data showing: A) Schematic representation of the destabilization domain (DD)-RIPK3 construct used. A DD-RIPK3 chimeric open reading frame was created by recombinant PCR, then cloned upstream of a T2A-GFP-T2A-PURO sequence. Of note, both DDRIPK3 and GFP protein include a C-terminal 2A epitope; B) NIH-3T3 cells stably expressing DD-RIPK3 were pre-treated with 1 μM Shield drug for 8 hours to stabilize RIPK3 expression, then treated with 1 ng/ml TNF and 50 μM zVAD as indicated. *P=0.0024; C) NIH-3T3 cells stably expressing DD-RIPK3 were treated with 1 μM Shield drug and 200 ng/ml TNFR-Fc as indicated. *p=0.0004; D) NIH-3T3 cells stably expressing catalytically inactive DD-RIPK3K51A were treated with 1 μM Shield drug as indicated. All cell death measurements were performed using an IncuCyte bioimager as described.

### DETAILED DESCRIPTION OF THE INVENTION

The present invention relates generally to compositions and methods for inducing necroptosis in a target cell. In particular, necroptosis is induced using compositions including oligomers comprising RIPK3 proteins and RIPK1 proteins including, but not limited to, full length RIPK3 homodimers, truncated RIPK3 oligomers and/or full length RIPK3/RIPK1 heterodimers. More specifically, the present invention relates to methods of inducing necroptosis in a target cell, including for example the cells of a tumor, upon the controlled formation of such homodimers, oligomers and/or heterodimers in vitro and in vivo.

The following is a listing of the exemplary sequences useful in the practice of the presently disclosed embodiments:

An amino acid sequence of a full length RIPK3 protein (Accession No. Q9Y572):

| (SEQ ID NO: 1) | |
|---|---|
| 1 | mscvklwpsg apaplvsiee lenqelvgkg gfgtvfraqh rkwgydvavk ivnskaisre |
| 61 | vkamasldne Fvlrlegvie kvnwdqdpkp alvtkfmeng slsgllqsqc prpwpllcrl |
| 121 | lkevvlgmfy lhdqnpvllh rdlkpsnvll dpelhvklad fglstfqggs qsgtgsgepg |
| 181 | gtlgylapel Fvnvnrkast asdvysfgil mwavlagrev elptepslvy eavcnrqnrp |
| 241 | slaelpqagp etpgleglke lmqlcwssep kdrpsfqecl pktdevfqmv ennmnaavst |
| 301 | vkdflsqlrs snrrfsipes gqggtemdgf rrtienqhsr ndvmvsewln klnleeppss |
| 361 | vpkkcpsltk rsraqeeqvp qawtagtssd smaqppqtpe tstfrnqmps ptstgtpspg |
| 421 | prgnqgaerq gmnwscrtpe pnpvtgrplv niyncsgvqv gdnnyltmqq ttalptwgla |
| 481 | psgkgrglqh pppvgsqegp kdpeawsrpq gwynhsgk |

(underlining = a RHIM domain).

An amino acid sequence of a truncated protein RIPK3^{ΔRHIM}:

| (SEQ ID NO: 2) | |
|---|---|
| 1 | mscvklwpsg apaplvsiee lenqelvgkg gfgtvfraqh rkwgydvavk ivnskaisre |
| 61 | vkamasldne Fvlrlegvie kvnwdqdpkp alvtkfmeng slsgllqsqc prpwpllcrl |
| 121 | lkevvlgmfy lhdqnpvllh rdlkpsnvll dpelhvklad fglstfqggs qsgtgsgepg |
| 181 | gtlgylapel Fvnvnrkast asdvysfgil mwavlagrev elptepslvy eavcnrqnrp |
| 241 | slaelpqagp etpgleglke lmqlcwssep kdrpsfqecl pktdevfqmv ennmnaavst |
| 301 | vkdflsqlrs snrrfsipes gqggtemdgf rrtienqhsr ndvmvsewln klnleeppss |
| 361 | vpkkcpsltk rsraqeeqvp qawtagtssd smaqppqtpe tstfrnqmps ptstgtpspg |
| 421 | prgnqgaerq gmnwscrtpe pnpvtgrplv niyncsaaa adnnyltmqq ttalptwg |
| 481 | psgkgrglqh pppvgsqegp kdpeawsrpq gwynhsgk |

(underlining = a mutated RHIM domain).

An amino acid sequence of a full length RIPK1 protein (Accession No. Q13546):

| (SEQ ID NO: 3) | |
|---|---|
| 1 | mqpdmslnvi kmkssdfles aeldsggfgk vslcfhrtqg lmimktvykg pnciehneal |
| 61 | leeakmmnrl rhsrvvkllg viieegkysl vmeymekgnl mhvlkaemst plsvkgriil |
| 121 | eiiegmcylh gkgvihkdlk penilvdndf hikiadlgla sfkmwsklnn eehnelrevd |
| 181 | gtakknggtl yymapehlnd vnakpteksd vysfavvlwa ifankepyen aiceqqlimc |
| 241 | iksgnrpdvd diteycprei islmklcwea npearptfpg ieekfrpfyl sqleesveed |
| 301 | vkslkkeysn enavvkrmqs lqldcvavps srsnsateqp gslhssqglg mgpveeswfa |
| 361 | pslehpqeen epslqsklqd eanyhlygsr mdrqtkqqpr qnvaynreee rrrrvshdpf |
| 421 | aqqrpyenfq ntegkgtays saashgnavh qpsgltsqpq vlyqnnglys shgfgtrpld |
| 481 | pgtagprvwy rpipshmpsl hnipvpetny lgntptmpfs slpptdesik ytiynstgiq |
| 541 | igaynymeig gtssslldst ntnfkeepaa kyqaifdntt sltdkhldpi renlgkhwkn |
| 601 | carklgftqs qideidhdye rdglkekvyq mlqkwvmreg ikgatvgkla qalhqcsrid |
| 661 | llssliyvsq n |

An amino acid sequence of a RHIM domain:

(SEQ ID NO: 4)

iqig

An amino acid sequence of a RHIM domain:

(SEQ ID NO: 5)

vqvg

An amino acid sequence of a modified FKBP protein (an Fv domain; F36V):

| (SEQ ID NO: 6) | |
|---|---|
| 001 | MASRGVQVET ISPGDGRTFP KRGQTCVVHY TGMLEDGKKV DSSRDRNKPF |
| 051 | KFMLGKQEVI RGWEEGVAQM SVGQRAKLTI SPDYAYGATG HPGIIPPHAT |
| 101 | LVFDVELLKL ETS* |

An amino acid sequence of an FRB protein:

| (SEQ ID NO: 7) | |
|---|---|
| 001 | MASRILWHEM WHEGLEEASR LYFGERNVKG MFEVLEPLHA MMERGPQTLK |
| 051 | ETSFNQAYGR DLMEAQEWCR KYMKSGNVKD LLQAWDLYYH VFRRISKTS* |

An amino acid sequence of a full length RIPK3 fusion protein (RIPK3-Fv):

| (SEQ ID NO: 8) | |
|---|---|
| 001 | MSCVKLWPSG APAPLVSIEE LENQELVGKG GFGTVFRAQH RKWGYDVAVK |
| 051 | IVNSKAISRE VKAMASLDNE FVLRLEGVIE KVNWDQDPKP ALVTKFMENG |
| 101 | SLSGLLQSQC PRPWPLLCRL LKEVVLGMFY LHDQNPVLLH RDLKPSNVLL |
| 151 | DPELHVKLAD FGLSTFQGGS QSGTGSGEPG GTLGYLAPEL FVNVNRKAST |
| 201 | ASDVYSFGIL MWAVLAGREV ELPTEPSLVY EAVCNRQNRP SLAELPQAGP |
| 251 | ETPGLEGLKE LMQLCWSSEP KDRPSFQECL PKTDEVFQMV ENNMNAAVST |
| 301 | VKDFLSQLRS SNRRFSIPES GQGGTEMDGF RRTIENQHSR NDVMVSEWLN |
| 351 | KLNLEEPPSS VPKKCPSLTK RSRAQEEQVP QAWTAGTSSD SMAQPPQTPE |
| 401 | TSTFRNQMPS PTSTGTPSPG PRGNQGAERQ GMNWSCRTPE PNPVTGRPLV |
| 451 | NIYNCSGVQVG DNNYLTMQQT TALPTWGLAP SGKGRGLQHP PPVGSQEGPK |
| 501 | DPEAWSRPQG WYNHSGKVAS RGVQVETISP GDGRTFPKRG QTCVVHYTGM |
| 551 | LEDGKKVDSS RDRNKPFKFM LGKQEVIRGW EEGVAQMSVG QRAKLTISPD |
| 601 | YAYGATGHPG IIPPHATLVF DVELLKLETS |

(underlining = RHIM domain; italics = Fv domain)

An amino acid sequence of a truncated fusion protein RIPK3^{ΔC}-2xFv:

| (SEQ ID NO: 9) | |
|---|---|
| 001 | MSCVKLWPSG APAPLVSIEE LENQELVGKG GFGTVFRAQH RKWGYDVAVK |
| 051 | IVNSKAISRE VKAMASLDNE FVLRLEGVIE KVNWDQDPKP ALVTKFMENG |
| 101 | SLSGLLQSQC PRPWPLLCRL LKEVVLGMFY LHDQNPVLLH RDLKPSNVLL |
| 151 | DPELHVKLAD FGLSTFQGGS QSGTGSGEPG GTLGYLAPEL FVNVNRKAST |
| 201 | ASDVYSFGIL MWAVLAGREV ELPTEPSLVY EAVCNRQNRP SLAELPQAGP |
| 251 | ETPGLEGLKE LMQLCWSSEP KDRPSFQECL PKTDEVFQMV ENNMNAAVST |
| 301 | VKDFLSQLRS SNRRFSIPES GQGGTEMDGF RRTIENQHSR NDVMVSEWLN |
| 351 | KLNLEEPPSS VPKKCPSLTK RSRAQEEQVP QAWTAGTSSD SMAQPPQTPE |
| 401 | TSTFRNQMPS PTSTGTPSPG PRGNQGAERQ GMNWSCRTPE PNPVTGRPLV |
| 451 | NIYGVQVETI SPGDGRTFPK RGQTCVVHYT GMLEDGKKVD SSRDRNKPFK |
| 501 | FMLGKQEVIR GWEEGVAQMS VGQRAKLTIS PDYAYGATGH PGIIPPHATL |
| 551 | VFDVELLKLE TRGVQVETIS PGDGRTFPKR GQTCVVHYTG MLEDGKKVDS |
| 601 | SRDRNKPFKF MLGKQEVIRG WEEGVAQMSV GQRAKLTISP DYAYGATGHP |
| 651 | GIIPPHATLV FDVELLKLET S* |

(italics = Fv domain)

An amino acid sequence of the full length RIPK1 fusion protein RIPK1-Fv:

(SEQ ID NO: 10)

MQPDMSLNVIKMKSSDFLESAELDSGGFGKVSLCFHRTQGLMIMKTVYKG
PNCIEHNEALLEEAKMMNRLRHSRVVKLLGVIIEEGKYSLVMEYMEKGNL
MHVLKAEMSTPLSVKGRIILEIIEGMCYLHGKGVIHKDLKPENILVDNDF
HIKIADLGLASFKMWSKLNNEEHNELREVDGTAKKNGGTLYYMAPEHLND
VNAKPTEKSDVYSFAVVLWAIFANKEPYENAICEQQLIMCIKSGNRPDVD
DITEYCPREIISLMKLCWEANPEARPTFPGIEEKFRPFYLSQLEESVEED
VKSLKKEYSNENAVVKRMQSLQLDCVAVPSSRSNSATEQPGSLHSSQGLG
MGPVEESWFAPSLEHPQEENEPSLQSKLQDEANYHLYGSRMDRQTKQQPR
QNVAYNREEERRRRVSHDPFAQQRPYENFQNTEGKGTAYSSAASHGNAVH
QPSGLTSQPQVLYQNNGLYSSHGFGTRPLDPGTAGPRVWYRPIPSHMPSL
HNIPVPETNYLGNTPTMPFSSLPPTDESIKYTIYNSTGIQIGAYNYMEIG
GTSSSLLDSTNTNFKEEPAAKYQAIFDNTTSLTDKHLDPIRENLGKHWKN
CARKLGFTQSQIDEIDHDYERDGLKEKVYQMLQKWVMREGIKGATVGKLA
QALHQCSRIDLLSSLIYVSQNMASRGVQVETISPGDGRTFPKRGQTCVVH
YTGMLEDGKKVDSSRDRNKPFKFMLGKQEVIRGWEEGVAQMSVGQRAKLT
ISPDYAYGATGHPGIIPPHATLVFDVELLKLETS*

A nucleic acid sequence of the full length RIPK3 polynucleotide:

(SEQ ID NO: 11)

ATGTCGTGCGTCAAGTTATGGCCCAGCGGTGCCCCCGCCCCCTTGGTGTC
CATCGAGGAACTGGAGAACCAGGAGCTCGTCGGCAAAGGCGGGTTCGGCA
CAGTGTTCCGGGCGCAACATAGGAAGTGGGGCTACGATGTGGCGGTCAAG
ATCGTAAACTCGAAGGCGATATCCAGGGAGGTCAAGGCCATGGCAAGTCT
GGATAACGAATTCGTGCTGCGCCTAGAAGGGGTTATCGAGAAGGTGAACT
GGGACCAAGATCCCAAGCCGGCTCTGGTGACTAAATTCATGGAGAACGGC
TCCTTGTCGGGGCTGCTGCAGTCCCAGTGCCCTCGGCCCTGGCCGCTCCT
TTGCCGCCTGCTGAAAGAAGTGGTGCTTGGGATGTTTTACCTGCACGACC
AGAACCCGGTGCTCCTGCACCGGGACCTCAAGCCATCCAACGTCCTGCTG
GACCCAGAGCTGCACGTCAAGCTGGCAGATTTTGGCCTGTCCACATTTCA
GGGAGGCTCACAGTCAGGGACAGGGTCCGGGGAGCCAGGGGGCACCCTGG
GCTACTTGGCCCCAGAACTGTTTGTTAACGTAAACCGGAAGGCCTCCACA
GCCAGTGACGTCTACAGCTTCGGGATCCTAATGTGGGCAGTGCTTGCTGG
AAGAGAAGTTGAGTTGCCAACCGAACCATCACTCGTGTACGAAGCAGTGT
GCAACAGGCAGAACCGGCCTTCATTGGCTGAGCTGCCCCAAGCCGGGCCT
GAGACTCCCGGCTTAGAAGGACTGAAGGAGCTAATGCAGCTCTGCTGGAG
CAGTGAGCCCAAGGACAGACCCTCCTTCCAGGAATGCCTACCAAAAACTG
ATGAAGTCTTCCAGATGGTGGAGAACAATATGAATGCTGCTGTCTCCACG
GTAAAGGATTTCCTGTCTCAGCTCAGGAGCAGCAATAGGAGATTTTCTAT
CCCAGAGTCAGGCCAAGGAGGGACAGAAATGGATGGCTTTAGGAGAACCA
TAGAAAACCAGCACTCTCGTAATGATGTCATGGTTTCTGAGTGGCTAAAC
AAACTGAATCTAGAGGAGCCTCCCAGCTCTGTTCCTAAAAAATGCCCGAG
CCTTACCAAGAGGAGCAGGGCACAAGAGGAGCAGGTTCCACAAGCCTGGA
CAGCAGGCACATCTTCAGATTCGATGGCCCAACCTCCCCAGACTCCAGAG
ACCTCAACTTTCAGAAACCAGATGCCCAGCCCTACCTCAACTGGAACACC
AAGTCCTGGACCCCGAGGGAATCAGGGGGCTGAGAGACAAGGCATGAACT
GGTCCTGCAGGACCCCGGAGCCAAATCCAGTAACAGGGCGACCGCTCGTT
AACATATACAACTGCTCTGGGGTGCAAGTTGGAGACAACAACTACTTGAC
TATGCAACAGACAACTGCCTTGCCCACATGGGGCTTGGCACCTTCGGGCA
AGGGGAGGGGCTTGCAGCACCCCCCACCAGTAGGTTCGCAAGAAGGCCCT
AAAGATCCTGAAGCCTGGAGCAGGCCACAGGGTTGGTATAATCATAGCGG
GAAATAA

A nucleic acid sequence of a truncated RIPK3^{ΔC} polynucleotide:

(SEQ ID NO: 12)

ATGTCGTGCGTCAAGTTATGGCCCAGCGGTGCCCCCGCCCCCTTGGTGTC
CATCGAGGAACTGGAGAACCAGGAGCTCGTCGGCAAAGGCGGGTTCGGCA
CAGTGTTCCGGGCGCAACATAGGAAGTGGGGCTACGATGTGGCGGTCAAG
ATCGTAAACTCGAAGGCGATATCCAGGGAGGTCAAGGCCATGGCAAGTCT
GGATAACGAATTCGTGCTGCGCCTAGAAGGGGTTATCGAGAAGGTGAACT
GGGACCAAGATCCCAAGCCGGCTCTGGTGACTAAATTCATGGAGAACGGC
TCCTTGTCGGGGCTGCTGCAGTCCCAGTGCCCTCGGCCCTGGCCGCTCCT
TTGCCGCCTGCTGAAAGAAGTGGTGCTTGGGATGTTTTACCTGCACGACC
AGAACCCGGTGCTCCTGCACCGGGACCTCAAGCCATCCAACGTCCTGCTG
GACCCAGAGCTGCACGTCAAGCTGGCAGATTTTGGCCTGTCCACATTTCA
GGGAGGCTCACAGTCAGGGACAGGGTCCGGGGAGCCAGGGGGCACCCTGG
GCTACTTGGCCCCAGAACTGTTTGTTAACGTAAACCGGAAGGCCTCCACA
GCCAGTGACGTCTACAGCTTCGGGATCCTAATGTGGGCAGTGCTTGCTGG
AAGAGAAGTTGAGTTGCCAACCGAACCATCACTCGTGTACGAAGCAGTGT
GCAACAGGCAGAACCGGCCTTCATTGGCTGAGCTGCCCCAAGCCGGGCCT
GAGACTCCCGGCTTAGAAGGACTGAAGGAGCTAATGCAGCTCTGCTGGAG
CAGTGAGCCCAAGGACAGACCCTCCTTCCAGGAATGCCTACCAAAAACTG
ATGAAGTCTTCCAGATGGTGGAGAACAATATGAATGCTGCTGTCTCCACG
GTAAAGGATTTCCTGTCTCAGCTCAGGAGCAGCAATAGGAGATTTTCTAT
CCCAGAGTCAGGCCAAGGAGGGACAGAAATGGATGGCTTTAGGAGAACCA
TAGAAAACCAGCACTCTCGTAATGATGTCATGGTTTCTGAGTGGCTAAAC
AAACTGAATCTAGAGGAGCCTCCCAGCTCTGTTCCTAAAAAATGCCCGAG
CCTTACCAAGAGGAGCAGGGCACAAGAGGAGCAGGTTCCACAAGCCTGGA
CAGCAGGCACATCTTCAGATTCGATGGCCCAACCTCCCCAGACTCCAGAG
ACCTCAACTTTCAGAAACCAGATGCCCAGCCCTACCTCAACTGGAACACC
AAGTCCTGGACCCCGAGGGAATCAGGGGGCTGAGAGACAAGGCATGAACT
GGTCCTGCAGGACCCCGGAGCCAAATCCAGTAACAGGGCGACCGCTCGTT
AACATATAC

A nucleic acid sequence of a full length RIPK1 polynucleotide:

(SEQ ID NO: 13)

ATGCAACCAGACATGTCCTTGAATGTCATTAAGATGAAATCCAGTGACTT
CCTGGAGAGTGCAGAACTGGACAGCGGAGGCTTTGGGAAGGTGTCTCTGT
GTTTCCACAGAACCCAGGGACTCATGATCATGAAAACAGTGTACAAGGGG
CCCAACTGCATTGAGCACAACGAGGCCCTCTTGGAGGAGGCGAAGATGAT
GAACAGACTGAGACACAGCCGGGTGGTGAAGCTCCTGGGCGTCATCATAG
AGGAAGGGAAGTACTCCCTGGTGATGGAGTACATGGAGAAGGGCAACCTG
ATGCACGTGCTGAAAGCCGAGATGAGTACTCCGCTTTCTGTAAAAGGAAG
GATAATTTTGGAAATCATTGAAGGAATGTGCTACTTACATGGAAAAGGCG
TGATACACAAGGACCTGAAGCCTGAAAATATCCTTGTTGATAATGACTTC
CACATTAAGATCGCAGACCTCGGCCTTGCCTCCTTTAAGATGTGGAGCAA
ACTGAATAATGAAGAGCACAATGAGCTGAGGGAAGTGGACGGCACCGCTA
AGAAGAATGGCGGCACCCTCTACTACATGGCGCCCGAGCACCTGAATGAC
GTCAACGCAAAGCCCACAGAGAAGTCGGATGTGTACAGCTTTGCTGTAGT
ACTCTGGGCGATATTTGCAAATAAGGAGCCATATGAAAATGCTATCTGTG
AGCAGCAGTTGATAATGTGCATAAAATCTGGGAACAGGCCAGATGTGGAT
GACATCACTGAGTACTGCCCAAGAGAAATTATCAGTCTCATGAAGCTCTG
CTGGGAAGCGAATCCGGAAGCTCGGCCGACATTTCCTGGCATTGAAGAAA
AATTTAGGCCTTTTTATTTAAGTCAATTAGAAGAAAGTGTAGAAGAGGAC
GTGAAGAGTTTAAAGAAAGAGTATTCAAACGAAAATGCAGTTGTGAAGAG
AATGCAGTCTCTTCAACTTGATTGTGTGGCAGTACCTTCAAGCCGGTCAA
ATTCAGCCACAGAACAGCCTGGTTCACTGCACAGTTCCCAGGGACTTGGG
ATGGGTCCTGTGGAGGAGTCCTGGTTTGCTCCTTCCCTGGAGCACCCACA
AGAAGAGAATGAGCCCAGCCTGCAGAGTAAACTCCAAGACGAAGCCAACT
ACCATCTTTATGGCAGCCGCATGGACAGGCAGACGAAACAGCAGCCCAGA
CAGAATGTGGCTTACAACAGAGAGGAGGAAAGGAGACGCAGGGTCTCCCA
TGACCCTTTTGCACAGCAAAGACCTTACGAGAATTTTCAGAATACAGAGG
GAAAAGGCACTGCTTATTCCAGTGCAGCCAGTCATGGTAATGCAGTGCAC
CAGCCCTCAGGGCTCACCAGCCAACCTCAAGTACTGTATCAGAACAATGG
ATTATATAGCTCACATGGCTTTGGAACAAGACCACTGGATCCAGGAACAG
CAGGTCCCAGAGTTTGGTACAGGCCAATTCCAAGTCATATGCCTAGTCTG
CATAATATCCCAGTGCCTGAGACCAACTATCTAGGAAATACACCCACCAT
GCCATTCAGCTCCTTGCCACCAACAGATGAATCTATAAAATATACCATAT
ACAATAGTACTGGCATTCAGATTGGAGCCTACAATTATATGGAGATTGGT
GGGACGAGTTCATCACTACTAGACAGCACAAATACGAACTTCAAAGAAGA
GCCAGCTGCTAAGTACCAAGCTATCTTTGATAATACCACTAGTCTGACGG
ATAAACACCTGGACCCAATCAGGGAAAATCTGGGAAAGCACTGGAAAAAC
TGTGCCCGTAAACTGGGCTTCACACAGTCTCAGATTGATGAAATTGACCA
TGACTATGAGCGAGATGGACTGAAAGAAAAGGTTTACCAGATGCTCCAAA
AGTGGGTGATGAGGGAAGGCATAAAGGGAGCCACGGTGGGGAAGCTGGCC
CAGGCGCTCCACCAGTGTTCCAGGATCGACCTTCTGAGCAGCTTGATTTA
CGTCAGCCAGAACTAA

A nucleic acid sequence of an RHIM domain:

(SEQ ID NO: 14)

GTGCAAGTTGGA

A nucleic acid sequence of an RHIM domain:

(SEQ ID NO: 15)

ATTCAGATTGGA

A nucleic acid sequence of an Fv polynucleotide:

(SEQ ID NO: 16)

ATGGCTTCTAGAGGAGTGCAGGTGGAGACTATCTCCCCAGGAGACGGGCG
CACCTTCCCCAAGCGCGGCCAGACCTGCGTGGTGCACTACACCGGGATGC
TTGAAGATGGAAAGAAAGTTGATTCCTCCCGGGACAGAAACAAGCCCTTT
AAGTTTATGCTAGGCAAGCAGGAGGTGATCCGAGGCTGGGAAGAAGGGGT
TGCCCAGATGAGTGTGGGTCAGAGAGCCAAACTGACTATATCTCCAGATT
ATGCCTATGGTGCCACTGGGCACCCAGGCATCATCCCACCACATGCCACT
CTCGTCTTCGATGTGGAGCTTCTAAAACTGGAAACTAGT

A nucleic acid sequence of an FRB polynucleotide:

(SEQ ID NO: 17)

ATGGCTTCTAGAATCCTCTGGCATGAGATGTGGCATGAAGGCCTGGAAGA
GGCATCTCGTTTGTACTTTGGGGAAAGGAACGTGAAAGGCATGTTTGAGG
TGCTGGAGCCCTTGCATGCTATGATGGAACGGGGCCCCCAGACTCTGAAG
GAAACATCCTTTAATCAGGCCTATGGTCGAGATTTAATGGAGGCCCAAGA
GTGGTGCAGGAAGTACATGAAATCAGGGAATGTCAAGGACCTCCTCCAAG
CCTGGGACCTCTATTATCATGTGTTCCGACGAATCTCAAAGACTAGTTAT
CCGTACGACGTACCAGACTACGCA

A nucleic acid sequence of a full length RIPK3-Fv polynucleotide:

(SEQ ID NO: 18)

ATGTCGTGCGTCAAGTTATGGCCCAGCGGTGCCCCCGCCCCCTTGGTGTC
CATCGAGGAACTGGAGAACCAGGAGCTCGTCGGCAAAGGCGGGTTCGGCA
CAGTGTTCCGGGCGCAACATAGGAAGTGGGGCTACGATGTGGCGGTCAAG
ATCGTAAACTCGAAGGCGATATCCAGGGAGGTCAAGGCCATGGCAAGTCT
GGATAACGAATTCGTGCTGCGCCTAGAAGGGGTTATCGAGAAGGTGAACT
GGGACCAAGATCCCAAGCCGGCTCTGGTGACTAAATTCATGGAGAACGGC
TCCTTGTCGGGGCTGCTGCAGTCCCAGTGCCCTCGGCCCTGGCCGCTCCT
TTGCCGCCTGCTGAAAGAAGTGGTGCTTGGGATGTTTTACCTGCACGACC
AGAACCCGGTGCTCCTGCACCGGGACCTCAAGCCATCCAACGTCCTGCTG
GACCCAGAGCTGCACGTCAAGCTGGCAGATTTTGGCCTGTCCACATTTCA
GGGAGGCTCACAGTCAGGGACAGGGTCCGGGGAGCCAGGGGGCACCCTGG
GCTACTTGGCCCCAGAACTGTTTGTTAACGTAAACCGGAAGGCCTCCACA
GCCAGTGACGTCTACAGCTTCGGGATCCTAATGTGGGCAGTGCTTGCTGG
AAGAGAAGTTGAGTTGCCAACCGAACCATCACTCGTGTACGAAGCAGTGT
GCAACAGGCAGAACCGGCCTTCATTGGCTGAGCTGCCCCAAGCCGGGCCT
GAGACTCCCGGCTTAGAAGGACTGAAGGAGCTAATGCAGCTCTGCTGGAG
CAGTGAGCCCAAGGACAGACCCTCCTTCCAGGAATGCCTACCAAAAACTG
ATGAAGTCTTCCAGATGGTGGAGAACAATATGAATGCTGCTGTCTCCACG
GTAAAGGATTTCCTGTCTCAGCTCAGGAGCAGCAATAGGAGATTTTCTAT
CCCAGAGTCAGGCCAAGGAGGGACAGAAATGGATGGCTTTAGGAGAACCA
TAGAAAACCAGCACTCTCGTAATGATGTCATGGTTTCTGAGTGGCTAAAC
AAACTGAATCTAGAGGAGCCTCCCAGCTCTGTTCCTAAAAAATGCCCGAG
CCTTACCAAGAGGAGCAGGGCACAAGAGGAGCAGGTTCCACAAGCCTGGA
CAGCAGGCACATCTTCAGATTCGATGGCCCAACCTCCCCAGACTCCAGAG
ACCTCAACTTTCAGAAACCAGATGCCCAGCCCTACCTCAACTGGAACACC
AAGTCCTGGACCCCGAGGGAATCAGGGGGCTGAGAGACAAGGCATGAACT
GGTCCTGCAGGACCCCGGAGCCAAATCCAGTAACAGGGCGACCGCTCGTT
AACATATACAACTGCTCTGGGGTGCAAGTTGGAGACAACAACTACTTGAC
TATGCAACAGACAACTGCCTTGCCCACATGGGGCTTGGCACCTTCGGGCA
AGGGGAGGGGCTTGCAGCACCCCCCACCAGTAGGTTCGCAAGAAGGCCCT
AAAGATCCTGAAGCCTGGAGCAGGCCACAGGGTTGGTATAATCATAGCGG
GAAAgTGGCTTCTAGAGGAGTGCAGGTGGAGACTATCTCCCCAGGAGACG
GGCGCACCTTCCCCAAGCGCGGCCAGACCTGCGTGGTGCACTACACCGGG
ATGCTTGAAGATGGAAAGAAAGTTGATTCCTCCCGGGACAGAAACAAGCC
CTTTAAGTTTATGCTAGGCAAGCAGGAGGTGATCCGAGGCTGGGAAGAAG
GGGTTGCCCAGATGAGTGTGGGTCAGAGAGCCAAACTGACTATATCTCCA
GATTATGCCTATGGTGCCACTGGGCACCCAGGCATCATCCCACCACATGC
CACTCTCGTCTTCGATGTGGAGCTTCTAAAACTGGAAACTAGT

A nucleic acid sequence of a truncated RIPK3^{ΔC}-2xFv polynucleotide:

(SEQ ID NO: 19)

ATGTCGTGCGTCAAGTTATGGCCCAGCGGTGCCCCCGCCCCCTTGGTGTC
CATCGAGGAACTGGAGAACCAGGAGCTCGTCGGCAAAGGCGGGTTCGGCA
CAGTGTTCCGGGCGCAACATAGGAAGTGGGGCTACGATGTGGCGGTCAAG
ATCGTAAACTCGAAGGCGATATCCAGGGAGGTCAAGGCCATGGCAAGTCT
GGATAACGAATTCGTGCTGCGCCTAGAAGGGGTTATCGAGAAGGTGAACT
GGGACCAAGATCCCAAGCCGGCTCTGGTGACTAAATTCATGGAGAACGGC
TCCTTGTCGGGGCTGCTGCAGTCCCAGTGCCCTCGGCCCTGGCCGCTCCT
TTGCCGCCTGCTGAAAGAAGTGGTGCTTGGGATGTTTTACCTGCACGACC
AGAACCCGGTGCTCCTGCACCGGGACCTCAAGCCATCCAACGTCCTGCTG
GACCCAGAGCTGCACGTCAAGCTGGCAGATTTTGGCCTGTCCACATTTCA
GGGAGGCTCACAGTCAGGGACAGGGTCCGGGGAGCCAGGGGGCACCCTGG
GCTACTTGGCCCCAGAACTGTTTGTTAACGTAAACCGGAAGGCCTCCACA
GCCAGTGACGTCTACAGCTTCGGGATCCTAATGTGGGCAGTGCTTGCTGG
AAGAGAAGTTGAGTTGCCAACCGAACCATCACTCGTGTACGAAGCAGTGT
GCAACAGGCAGAACCGGCCTTCATTGGCTGAGCTGCCCCAAGCCGGGCCT
GAGACTCCCGGCTTAGAAGGACTGAAGGAGCTAATGCAGCTCTGCTGGAG
CAGTGAGCCCAAGGACAGACCCTCCTTCCAGGAATGCCTACCAAAAACTG
ATGAAGTCTTCCAGATGGTGGAGAACAATATGAATGCTGCTGTCTCCACG
GTAAAGGATTTCCTGTCTCAGCTCAGGAGCAGCAATAGGAGATTTTCTAT
CCCAGAGTCAGGCCAAGGAGGGACAGAAATGGATGGCTTTAGGAGAACCA
TAGAAAACCAGCACTCTCGTAATGATGTCATGGTTTCTGAGTGGCTAAAC
AAACTGAATCTAGAGGAGCCTCCCAGCTCTGTTCCTAAAAAATGCCCGAG
CCTTACCAAGAGGAGCAGGGCACAAGAGGAGCAGGTTCCACAAGCCTGGA
CAGCAGGCACATCTTCAGATTCGATGGCCCAACCTCCCCAGACTCCAGAG
ACCTCAACTTTCAGAAACCAGATGCCCAGCCCTACCTCAACTGGAACACC
AAGTCCTGGACCCCGAGGGAATCAGGGGGCTGAGAGACAAGGCATGAACT
GGTCCTGCAGGACCCCGGAGCCAAATCCAGTAACAGGGCGACCGCTCGTT
AACATATACGGCGTCCAAGTCGAAACCATTAGTCCCGGCGATGGCAGAAC
ATTTCCTAAAAGGGGACAAACATGTGTCGTCCATTATACAGGCATGTTGG
AGGACGGCAAAAAGGTGGACAGTAGTAGAGATCGCAATAAACCTTTCAAA
TTCATGTTGGGAAAACAAGAAGTCATTAGGGGATGGGAGGAGGGCGTGGC
TCAAATGTCCGTCGGCCAACGCGCTAAGCTCACCATCAGCCCCGACTACG
CATACGGCGCTACCGGACATCCCGGAATTATTCCCCCTCACGCTACCTTG
GTGTTTGACGTCGAACTGTTGAAGCTCGAGACTAGAGGAGTGCAGGTGGA
GACTATCTCCCCAGGAGACGGGCGCACCTTCCCCAAGCGCGGCCAGACCT
GCGTGGTGCACTACACCGGGATGCTTGAAGATGGAAAGAAAGTTGATTCC
TCCCGGGACAGAAACAAGCCCTTTAAGTTTATGCTAGGCAAGCAGGAGGT
GATCCGAGGCTGGGAAGAAGGGGTTGCCCAGATGAGTGTGGGTCAGAGAG
CCAAACTGACTATATCTCCAGATTATGCCTATGGTGCCACTGGGCACCCA
GGCATCATCCCACCACATGCCACTCTCGTCTTCGATGTGGAGCTTCTAAA
ACTGGAAACTAGTTAA

A nucleic sequence of a full length RIPK1-Fv polynucleotide:

(SEQ ID NO: 20)

ATGCAACCAGACATGTCCTTGAATGTCATTAAGATGAAATCCAGTGACTT
CCTGGAGAGTGCAGAACTGGACAGCGGAGGCTTTGGGAAGGTGTCTCTGT
GTTTCCACAGAACCCAGGGACTCATGATCATGAAAACAGTGTACAAGGGG
CCCAACTGCATTGAGCACAACGAGGCCCTCTTGGAGGAGGCGAAGATGAT
GAACAGACTGAGACACAGCCGGGTGGTGAAGCTCCTGGGCGTCATCATAG
AGGAAGGGAAGTACTCCCTGGTGATGGAGTACATGGAGAAGGGCAACCTG
ATGCACGTGCTGAAAGCCGAGATGAGTACTCCGCTTTCTGTAAAAGGAAG
GATAATTTTGGAAATCATTGAAGGAATGTGCTACTTACATGGAAAAGGCG
TGATACACAAGGACCTGAAGCCTGAAAATATCCTTGTTGATAATGACTTC
CACATTAAGATCGCAGACCTCGGCCTTGCCTCCTTTAAGATGTGGAGCAA
ACTGAATAATGAAGAGCACAATGAGCTGAGGGAAGTGGACGGCACCGCTA
AGAAGAATGGCGGCACCCTCTACTACATGGCGCCCGAGCACCTGAATGAC
GTCAACGCAAAGCCCACAGAGAAGTCGGATGTGTACAGCTTTGCTGTAGT
ACTCTGGGCGATATTTGCAAATAAGGAGCCATATGAAAATGCTATCTGTG
AGCAGCAGTTGATAATGTGCATAAAATCTGGGAACAGGCCAGATGTGGAT
GACATCACTGAGTACTGCCCAAGAGAAATTATCAGTCTCATGAAGCTCTG
CTGGGAAGCGAATCCGGAAGCTCGGCCGACATTTCCTGGCATTGAAGAAA
AATTTAGGCCTTTTTATTTAAGTCAATTAGAAGAAAGTGTAGAAGAGGAC
GTGAAGAGTTTAAAGAAAGAGTATTCAAACGAAAATGCAGTTGTGAAGAG
AATGCAGTCTCTTCAACTTGATTGTGTGGCAGTACCTTCAAGCCGGTCAA
ATTCAGCCACAGAACAGCCTGGTTCACTGCACAGTTCCCAGGGACTTGGG
ATGGGTCCTGTGGAGGAGTCCTGGTTTGCTCCTTCCCTGGAGCACCCACA
AGAAGAGAATGAGCCCAGCCTGCAGAGTAAACTCCAAGACGAAGCCAACT
ACCATCTTTATGGCAGCCGCATGGACAGGCAGACGAAACAGCAGCCCAGA
CAGAATGTGGCTTACAACAGAGAGGAGGAAAGGAGACGCAGGGTCTCCCA
TGACCCTTTTGCACAGCAAAGACCTTACGAGAATTTTCAGAATACAGAGG
GAAAAGGCACTGCTTATTCCAGTGCAGCCAGTCATGGTAATGCAGTGCAC
CAGCCCTCAGGGCTCACCAGCCAACCTCAAGTACTGTATCAGAACAATGG
ATTATATAGCTCACATGGCTTTGGAACAAGACCACTGGATCCAGGAACAG
CAGGTCCCAGAGTTTGGTACAGGCCAATTCCAAGTCATATGCCTAGTCTG
CATAATATCCCAGTGCCTGAGACCAACTATCTAGGAAATACACCCACCAT
GCCATTCAGCTCCTTGCCACCAACAGATGAATCTATAAAATATACCATAT
ACAATAGTACTGGCATTCAGATTGGAGCCTACAATTATATGGAGATTGGT
GGGACGAGTTCATCACTACTAGACAGCACAAATACGAACTTCAAAGAAGA
GCCAGCTGCTAAGTACCAAGCTATCTTTGATAATACCACTAGTCTGACGG
ATAAACACCTGGACCCAATCAGGGAAAATCTGGGAAAGCACTGGAAAAAC
TGTGCCCGTAAACTGGGCTTCACACAGTCTCAGATTGATGAAATTGACCA
TGACTATGAGCGAGATGGACTGAAAGAAAAGGTTTACCAGATGCTCCAAA
AGTGGGTGATGAGGGAAGGCATAAAGGGAGCCACGGTGGGGAAGCTGGCC
CAGGCGCTCCACCAGTGTTCCAGGATCGACCTTCTGAGCAGCTTGATTTA
CGTCAGCCAGAACATGGCTTCTAGAGGAGTGCAGGTGGAGACTATCTCCC
CAGGAGACGGGCGCACCTTCCCCAAGCGCGGCCAGACCTGCGTGGTGCAC
TACACCGGGATGCTTGAAGATGGAAAGAAAGTTGATTCCTCCCGGGACAG
AAACAAGCCCTTTAAGTTTATGCTAGGCAAGCAGGAGGTGATCCGAGGCT
GGGAAGAAGGGGTTGCCCAGATGAGTGTGGGTCAGAGAGCCAAACTGACT
ATATCTCCAGATTATGCCTATGGTGCCACTGGGCACCCAGGCATCATCCC
ACCACATGCCACTCTCGTCTTCGATGTGGAGCTTCTAAAACTGGAAACTA
GTTAA

An amino acid sequence of an FK506 binding protein (Accession No. AAD40379):

| (SEQ ID NO: 21) | |
|---|---|
| 1 | mpktmhflfr fivffylwgl ftaqrqkkee steevkievl hrpencskts kkgdllnahy |
| 61 | dgylakdgsk fycsrtqneg hpkwFvlgvg qvikgldiam tdmcpgekrk vvippsfayg |
| 121 | keghaegkip pdatlifeie lyavtkgprs ietfkqidmd ndrqlskaei nlylqrefek |
| 181 | dekprdksyq davledifkk ndhdgdgfis pkeynvyqhd el |

An nucleic acid sequence of an FK506 binding protein (Accession No. AF092137):

| (SEQ ID NO: 22) | |
|---|---|
| 1 | ctagaattca gcggccgctt tttttctaga attcagcgcc gctgaattcc acgcgggagg |
| 61 | gagagcagtg ttctgctgga gccgatgcca aaaaccatgc atttcttatt cagattcatt |
| 121 | gttttctttt atctgtgggg cctttttact gctcagagac aaaagaaaga ggagagcacc |
| 181 | gaagaagtga aaatagaagt tttgcatcgt ccagaaaact gctctaagac aagcaagaag |
| 241 | ggagacctac taaatgccca ttatgacggc tacctggcta aagacggctc gaaattctac |
| 301 | tgcagccgga cacaaaatga aggccacccc aaatggtttg ttcttggtgt tgggcaagtc |
| 361 | ataaaaggcc tagacattgc tatgacagat atgtgccctg gagaaaagcg aaaagtagtt |
| 421 | ataccccctt catttgcata cggaaaggaa ggccatgcag aaggcaagat tccaccggat |
| 481 | gctacattga tttttgagat tgaactttat gctgtgacca aaggaccacg gagcattgag |
| 541 | acatttaaac aaatagacat ggacaatgac aggcagctct ctaaagccga gataaacctc |
| 601 | tacttgcaaa gggaatttga aaaagatgag aagccacgtg acaagtcata tcaggatgca |
| 661 | gttttagaag atatttttaa gaagaatgac catgatggtg atggcttcat ttctcccaag |
| 721 | gaatacaatg tataccaaca cgatgaacta tagcatattt gtatttctac tttttttttt |
| 781 | tagctattta ctgtacttta tgtataaaac aaagtcactt ttctccaagt tgtatttgct |
| 841 | atttttcccc tatgagaaga tattttgatc tccccaatac attgattttg gtataataaa |
| 901 | tgtgaggctg ttttgcaaac ttaacttgca ggaatggtat cgactcgtgt ttcctactgc |
| 961 | tttattctgt aaacaagaat tgtagcacca tgaaacagac ctctgggtcc cagtgggcat |
| 1021 | tttttcccct ttcaggatgt aggaggacat gtatagtatg tcaaaaactg caagcttttc |
| 1081 | ccaactttaa ccttaccagc atgttaatat ccagtttttt tatagtttaa aagttaaagt |
| 1141 | gcctcatatt ttgaaaatat ccattaagga cccaggaatt agcatttcac ttgtttatac |
| 1201 | atttttataa cattatgaag acgatataaa a |

An amino acid sequence of a truncated protein RIPK3^{ΔC}:

| (SEQ ID NO: 23) | |
|---|---|
| 1 | mscvklwpsg apaplvsiee lenqelvgkg gfgtvfraqh rkwgydvavk ivnskaisre |
| 61 | vkamasldne Fvlrlegvie kvnwdqdpkp alvtkfmeng slsgllqsqc prpwpllcrl |
| 121 | lkevvlgmfy lhdqnpvllh rdlkpsnvll dpelhvklad fglstfqggs qsgtgsgepg |
| 181 | gtlgylapel Fvnvnrkast asdvysfgil mwavlagrev elptepslvy eavcnrqnrp |
| 241 | slaelpqagp etpgleglke lmqlcwssep kdrpsfqecl pktdevfqmv ennmnaavst |
| 301 | vkdflsqlrs snrrfsipes gqggtemdgf rrtienqhsr ndvmvsewln klnleeppss |
| 361 | vpkkcpsltk rsraqeeqvp qawtagtssd smaqppqtpe tstfrnqmps ptstgtpspg |
| 421 | prgnqgaerq gmnwscrtpe pnpvtgrplv niyncs |

(Note: RHIM domain and C-terminal domain deleted)

An amino acid sequence of a truncated protein RIPK3^{ΔRHIM}-1xFv:

| (SEQ ID NO: 24) | |
|---|---|
| 1 | mscvklwpsg apaplvsiee lenqelvgkg gfgtvfraqh rkwgydvavk ivnskaisre |
| 61 | vkamasldne Fvlrlegvie kvnwdqdpkp alvtkfmeng slsgllqsqc prpwpllcrl |
| 121 | lkevvlgmfy lhdqnpvllh rdlkpsnvll dpelhvklad fglstfqggs qsgtgsgepg |
| 181 | gtlgylapel Fvnvnrkast asdvysfgil mwavlagrev elptepslvy eavcnrqnrp |
| 241 | slaelpqagp etpgleglke lmqlcwssep kdrpsfqecl pktdevfqmv ennmnaavst |
| 301 | vkdflsqlrs snrrfsipes gqggtemdgf rrtienqhsr ndvmvsewln klnleeppss |
| 361 | vpkkcpsltk rsraqeeqvp qawtagtssd smaqppqtpe tstfrnqmps ptstgtpspg |
| 421 | prgnqgaerq gmnwscrtpe pnpvtgrplv niyncsaaa adnnyltmqq ttalptwg |
| 481 | psgkgrglqh pppvgsqegp kdpeawsrpq gwynhsgk masrgvqvet ispgdgrtfp |
| 501 | krgqtcvvhy tgmledgkkv dssrdrnkpf kfmlgkqevi rgweegvaqm svgqraklti |
| 561 | spdyaygatg hpgiipphat lvfdvellkl ets |

(underlining: mutated RHIM domain; italics: Fv domain)

In one embodiment, the present invention contemplates using inducible protein interaction systems to induce necrotic apoptosis (necroptosis) by RIPK3 activation. For example, the formation of a RIPK3 dimer via a C-terminal dimerization domain, while not itself sufficient to activate RIPK3, is able to “seed” a RHIM-dependent complex whose propagation leads to RIPK3 activation. Although it is not necessary to understand the mechanism of an invention, it is believed that the stability of the RIPK3/RHIM-dependent complex, and by extension, the activation of RIPK3 and necroptosis may be controlled by caspase-8 and RIPK1. The data demonstrate that both caspase-8 and RIPK1 are recruited to facilitate RIPK3 oligomerization, as inhibition of caspase-8 potentiates RIPK3 oligomerization and necroptosis, while inhibition of RIPK1 inhibits RIPK3 oligomerization.

Surprisingly, however, while siRNA-mediated caspase-8 knockdown and chemical inhibition of caspase-8 has a similar effect on RIPK3 oligomerization and activation, siRNA-mediated RIPK1 knockdown and chemical inhibition of RIPK1 had opposing effects on RIPK3 oligomerization and activation. Specifically, RIPK1 inhibition reduced RIPK3 activation, while siRNA-mediated knockdown of RIPK1 notably potentiated it. In one embodiment, the present invention contemplates a method comprising inducing RIPK3 activation by modulating RIPK1 kinase activity. In one embodiment, the method further comprises inhibiting RIPK3 oligomer formation with RIPK1.

In one embodiment, the present invention contemplates a method of inducing necroptosis comprising dimerizing RIPK3 wherein an exposed RHIM domain recruits RIPK1 and/or RIPK3 into an amyloid-like oligomer. Although it is not necessary to understand the mechanism of an invention, it is believed that when RIPK1 is present in the oligomer, RIPK1 mediates the recruitment of caspase-8 in concert with cFLIPL. In one embodiment, the method further comprising destabilizing and/or inhibiting a necrosome. In one embodiment, the destabilizing and/or inhibiting may be mediated by interactions between a RIPK1 C-terminal death domain and an caspase-8 adapter (e.g., FADD). In one embodiment, an RIPK1 inhibitor (e.g., Nec1) promotes necrosome growth inhibition by effectively creating a catalytically inactive form of RIPK1 and recruiting inhibitory caspase-8. In one embodiment, an RIPK1 siRNA potentiates RIPK3 signaling by eliminating the suppressive bridging function.

Although it is not necessary to understand the mechanism of an invention, RHIM-dependent oligomerization of RIPK3 appears to play a role in RIPK3 activation that is further modulated by RIPK1 and caspase-8. It is also believed that chemically-enforced oligomerization potentiated RIPK3 activation and eliminated the ability of caspase-8 and RIPK1 to control this process.

### I. The Necrotic Pathway

In higher animals, necrosis is often viewed as an accidental and unregulated cellular event as compared to the developmental and homeostatic programmed cell death mediated by apoptosis. However, accumulating evidence suggests that necrosis, like apoptosis, can be executed by regulated mechanisms.

Apoptosis is defined by an ensemble of morphological features, including chromatin condensation and nuclear fragmentation, cell shrinkage, plasma membrane blebbing, and formation of apoptotic bodies. In contrast, necrosis fails to display a stereotyped morphology (except for the early rupture of plasma membranes) and has historically been regarded as an unregulated means of cell death that is induced by nonspecific and non-physiological stress (Kroemer et al., 2008).

Multiple lines of evidence now indicate the existence of a complex molecular pathway mediating programmed necrosis both in its occurrence and its mechanism:
1) necrotic cell death can contribute to embryonic development and adult tissue homeostasis,
2) necrotic cell death can be induced by ligands that bind to specific plasma membrane receptors, and
3) necrosis can be regulated by genetic, epigenetic, and pharmacological factors (Golstein and Kroemer, 2007).
   Moreover, the inactivation of caspases causes a shift from apoptosis either to, cell death morphologies with mixed necrotic and apoptotic features, or to full-blown necrosis (Kroemer et al., 2008). Such observations demonstrate that apoptotic and necrotic cell death modalities cross-regulate each other and substantiates the notion that necrosis is a cell death pathway that is unmasked when essential effectors of apoptosis are inhibited (Golstein and Kroemer, 2007).

The challenge of characterizing the precise mechanisms of programmed necrosis, as well as of the molecular switches between apoptosis and necrosis, has major therapeutic implications. In some instances, the selective inhibition of necrosis (and/or the facilitation of apoptotic cell death) may limit inflammation, and hence reduce secondary tissue damage. Conversely, it may be desirable to trigger the necrotic death of cancer cells that are resistant to apoptosis.

Necrotic cell death results from extensive crosstalk between several biochemical and molecular events at different cellular levels. Biochemica et Biosphysica Acta, 1757: 1371-2387 (2006). Recent data indicate that a serine/threonine kinase, RIPK1, which contains a death domain (DD), may act as a central initiator of necrotic cell death. Calcium and reactive oxygen species (ROS) are also players during the propagation and execution phases of necrotic cell death, directly or indirectly provoking damage to proteins, lipids and DNA, which culminates in disruption of organelle and cell integrity. Necrotically dying cells initiate pro-inflammatory signaling cascades by actively releasing inflammatory cytokines and by spilling their contents when they lyse.

While an understanding of the mechanism of the invention is not necessary, and without limiting the invention to any particular mechanism, it is known that death receptors belong to the TNF receptor superfamily. When they bind their extracellular ligands they aggregate and initiate a signaling pathway that results in either cell survival or death. Depending on the cellular context, cells die by apoptosis or necrosis. TNFα, a pleiotropic cytokine produced primarily by macrophages, induces apoptosis in many cells, but it can induce necrosis in the L929 mouse fibrosarcoma cell line. Addition of zVAD-fmk (a cell permeable pan-caspase inhibitor that irreversibly binds to the catalytic site of caspase proteases) or CrmA further sensitizes L929 cells to TNFα-induced necrotic cell death. Likewise, Fas ligand in the presence of zVAD-fmk leads to necrosis of this cell line. Similarly, the triggering of TNF-R1, Fas or TRAIL-R in Jurkat cells in the presence of zVAD-fmk or in Jurkat cells deficient in Fas-associated protein-containing death domain (FADD) or caspase-8 results in necrosis. In addition, TNFα in the presence of caspase inhibitors can induce caspase-independent cell death in murine embryonic fibroblasts (MEFs). In one embodiment, the present invention contemplates a method of inducing necrotic cell death following caspase inhibition and/or improper or partial activation of caspase-dependent pathways.

Apoptotic and necrotic signaling pathways have the FADD adaptor molecule in common. FADD contains both a death domain (DD) for initiating necrotic signaling, and a death effector domain (DED) that can propagate apoptotic cell death. In Fas and TRAIL-R-induced signaling, FADD may be recruited to the receptor and can initiate downstream signaling cascades, such as apoptosis and activation of NF-κB and MAPKs. Impeding caspase activation switches cell death from apoptosis to necrosis. In contrast to the triggering of TRAIL-R and Fas, engagement of TNF-R1 does not result in recruitment of FADD to the receptor. At the plasma membrane, formation of complex I, which consists of TNF-R1, TRAF2 and RIPK1, leads to rapid activation of NF-κB and MAPKs, such as p38 MAPK, JNK and ERK. Following receptor endocytosis, a second complex is formed, in which TRADD recruits FADD and procaspase-8 or -10. Endocytic vesicles fuse with trans-Golgi vesicles containing pro-acid-sphingomyelinase (pro-ASMase) and pre-pro-cathepsin D. This leads to formation of multivesicular endosomes in which acid-sphingomyelinase and cathepsin D are activated. If complex I does not succeed in inducing sufficient expression of antiapoptotic proteins, caspase-8 is activated, initiating apoptosis. However, if caspases are blocked, necrotic death ensues. The importance of FADD in TNFα-induced caspase-independent signaling is controversial: FADD seems necessary for TNFα-induced death in MEFs, but it is dispensable for TNFα-induced death of Jurkat cells. TNF-R2 is not essential for TNFα-induced necrosis but it seems to potentiate the process.

Studies in RIPK1^{-/-} Jurkat cells demonstrate that propagation of necrosis induced by triggering of Fas/TNF-R/TRAIL-R depends on the presence of RIPK1 kinase activity. Caspase-8 mediated cleavage of RIPK1 during TNF-R1, Fas and TRAIL-R-mediated apoptosis suppresses necrotic and anti-apoptotic pathways, also demonstrating that full-length RIPK1 is required for necrosis. Moreover, the C-terminal RIPK1 cleavage fragment containing the DD sensitizes cells to apoptosis by inhibiting NF-κB activation. Studies on the heat shock protein (Hsp) 90, a cytosolic chaperone for many kinases, including RIPK1, have also revealed the importance of RIPK1 in necrotic signaling. Fas- and TNF-R1-induced necrosis is inhibited by Hsp90 inhibitors geldanamycin (GA) and radicicol (RC), which are responsible for a strong down-regulation of RIPK1 levels. Moreover, knockdown of RIPK1 in L929 cells protects the cells against necrosis induced by TNFα/zVAD-fmk or FasL/zVAD-fmk.

Besides death receptor-induced necrosis, triggering of toll-like receptor (TLR) 4 and TLR3 can also lead to necrosis. Caspase-8 activation suppression by IETD-fmk, CrmA or zVAD-fmk, lipopolysaccharide (LPS) leads to an induction of an RIPK1-dependent, non-apoptotic death of macrophages. The presence of dsRNA in mammalian cells is a hallmark of viral infection, as most viruses produce dsRNA during their replication. Both viral and synthetic dsRNA were shown to kill cells, predominantly by FADD/caspase-8 mediated apoptosis. Synthetic dsRNA, however, induces necrosis in human Jurkat cells and murine L929 fibrosarcoma cells in a caspase-8 and FADD-independent way, and type I and II-interferons (IFNs) can sensitize for this necrosis.

Some pathophysiological processes, such as ischemia-reperfusion, inflammation, ROS-induced injury and glutamate excitotoxicity, are accompanied by poly-(ADP-ribose) polymerase-1 (PARP-1)-mediated cell death. Stimuli that directly or indirectly affect mitochondria, such as H₂O₂ and the DNA-alkylating agent N-methyl-N′-nitro-N-nitrosoguanidine (MNNG), also induce cell death mediated by PARP-1. Activation of PARP-1 catalyzes the hydrolysis of NAD⁺ into nicotinamide and poly-ADP ribose, causing depletion of NAD⁺. This results in cellular energy failure and caspase-independent death of different cell types. MNNG-induced cell death depends on RIPK1 and TRAF2, which function downstream of PARP-1 and are crucial for JNK activation. JNK in turn affects mitochondrial membrane integrity, with consequent release of proteins of the mitochondrial intermembrane space, and necrosis. It is not clear how JNK induces mitochondrial membrane depolarization, but it is plausible that it occurs through modifications of Bcl-2 family members (a family of regulatory proteins that regulate cell death), or via caspase-independent JNK-mediated processing of Bid. PARP mediated cell death induced by H₂O₂ also depends on a TRAF2/RIPK1/JNK-mediated signaling cascade.

Necroptosis is a form of programmed cell death that is both mechanistically and morphologically distinct from apoptosis (1a, 2a). While apoptosis is defined by the activation of the caspase proteases, necroptosis is triggered by receptor interacting protein kinase 1 (RIPK1) and RIPK3 (3a-7a). Morphologically, necroptosis resembles the unprogrammed process of necrosis, involving cellular swelling and rupture (8a). This morphology is distinct from apoptosis, in which dying cells shrink and their contents remain contained within membrane-bound bodies or vesicles. Necroptotic cell death thereby releases cellular contents that are contained during apoptosis; necroptosis is therefore thought to be an inflammatory form of cell death. Consistent with a proposed role in inflammation and immune responses, necroptosis can be triggered by TNF2, interferon or TLR signaling, as well as by viral infection via the DNA sensor DAI (9a-11a).

Necroptotic cell death plays a role in the host response to viral and bacterial infection, as well as the pathogenesis of TNF-induced sterile septic shock (12a-14a). The mechanism by which the necroptotic program is initiated has been studied principally in the context of TNFR1 activation, and it remains incompletely understood. Briefly, ligation of TNFR1 by TNF induces the assembly of a large receptor-proximal complex that includes RIPK1. Ubiquitination and phosphorylation events within this complex lead to activation of an NF-kB transcriptional program, and/or MAP kinase activation (15a).

Subsequently, RIPK1 is deubiquitinated and translocates into the cytosol, where it forms additional complexes that have been termed “necrosomes” or “ripoptosomes”; these scaffolds support RIPK3 activation, which in turn leads to phosphorylation of the downstream mediator MLKL and the process of necroptosis (16a-23a) Importantly, the cIAP ubiquitin ligases and the pro-apoptotic enzyme caspase-8, in concert with its paralog cFLIPL, can also be recruited to necrosome complexes, where they antagonize RIPK3 activation and necroptosis. (24a-26a)

The assembly and regulation of the RIPK1-RIPK3 necrosome is an open subject of investigation in the field. Recent structural analysis showed that RHIM domains of RIPK1 and RIPK3 form amyloid-like oligomers during RIPK3 activation. However, it remains unclear whether RIPK3 oligomerization, RHIM amyloid formation, or both are necessary and/or sufficient for RIPK3 activation. Furthermore, it is unclear how suppressors of necroptosis, such as caspase-8, interact with and regulate RIPK3 oligomers to determine cell fate.

Inducible protein interaction systems have provided fundamental insight into many cellular processes, including cell death. For example, versions of an FKBP-rapamycin interaction system has been used to create caspase proteases that could be induced to undergo homo- or heterodimerization by addition of specific drug ligands (27a-31a). Here, similar strategies were applied to determine the mechanism(s) of RIPK3 activation, with the goal of defining how its activation is regulated during cell life and in response to stress events that culminate in the induction of necroptosis. Using these systems, it was found that RIPK3 dimerization “seeds” RHIM-dependent oligomerization, the propagation of which is required for induction of necroptosis. This RHIM-dependent oligomerization is directly regulated by RIPK1 and caspase-8. Consistent with this model, the data presented herein demonstrate that chemically-enforced oligomerization of RIPK3 triggered potent cell death regardless of the presence of the RHIM domain, and that chemically-enforced oligomerization eliminated the ability of caspase-8 or RIPK1 to regulate RIPK3-dependent cell death.

Unexpectedly, the data demonstrate that while chemical inhibition of RIPK1 inhibited RHIM-dependent RIPK3 oligomerization and cell death, depletion of RIPK1 protein (e.g., by siRNA) in this system had an opposite effect. For example, in cells depleted in RIPK1, RIPK3 expression induced notably higher rates of spontaneous necroptosis. Together, these data indicate that RIPK3 oligomerization is both necessary and sufficient for the induction of necroptosis, and that RHIM-dependent oligomerization of RIPK3 recruits caspase-8 and RIPK1 for control of this process. Further, while RIPK1 is required for receptor-induced activation of RIPK3, it is shown herein that RIPK1 also exerts intrinsic suppression of RIPK3 oligomerization in the cytosol. In conclusion, the data thereby demonstrates that RIPK1 is a dual-function regulator of RIPK3, having implications for successful pharmacological targeting of these enzymes.

Preliminary results by others have used similar induced-interaction systems to define the minimal complex necessary for necroptotic signaling. Unlike the data presented herein, these preliminary results showed that chemically-enforced RIPK3 dimerization, even in the absence of a RHIM domain, was sufficient (but not necessary) to trigger RIPK3 autophosphorylation, MLKL activation and necroptosis. A likely explanation for this apparent discrepancy is that the dimerization domains were appended adjacent to the N-terminal kinase domain of RIPK3, instead of C-terminal dimerization domains in an effort to mimic the action of the C-terminal RHIM domain.

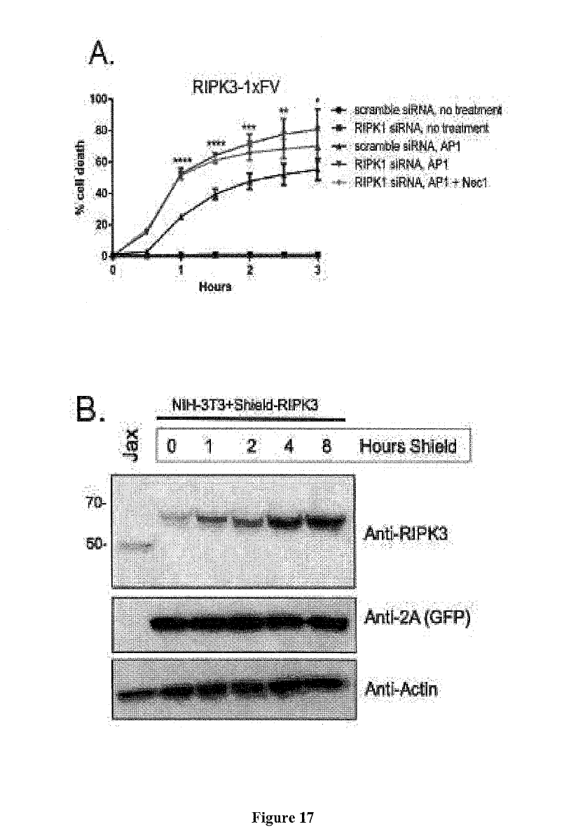

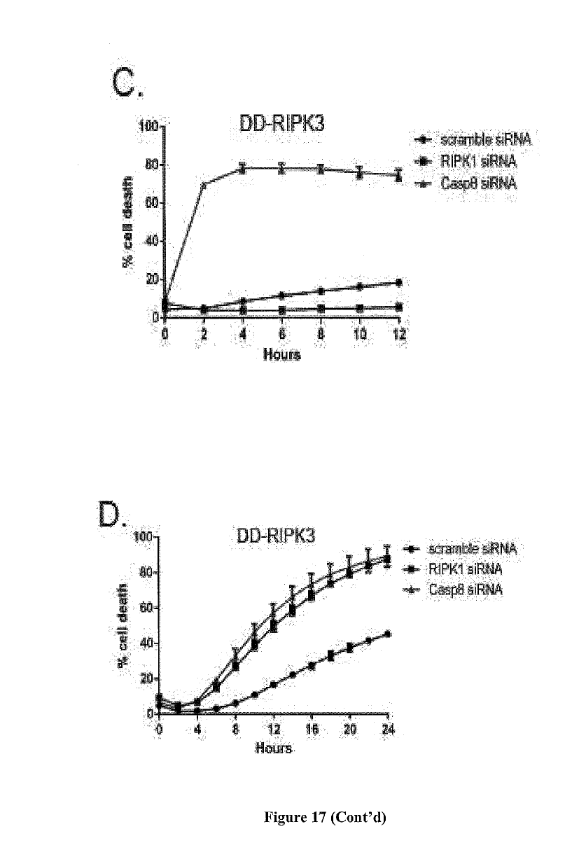

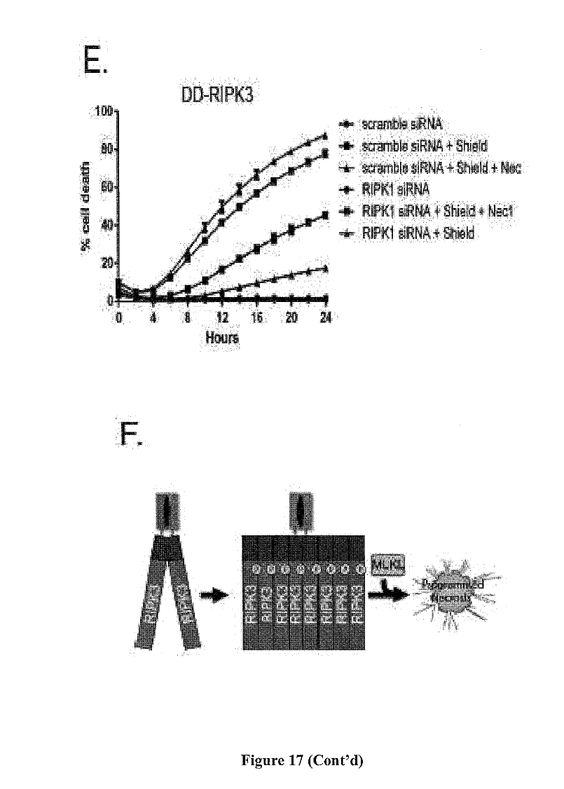

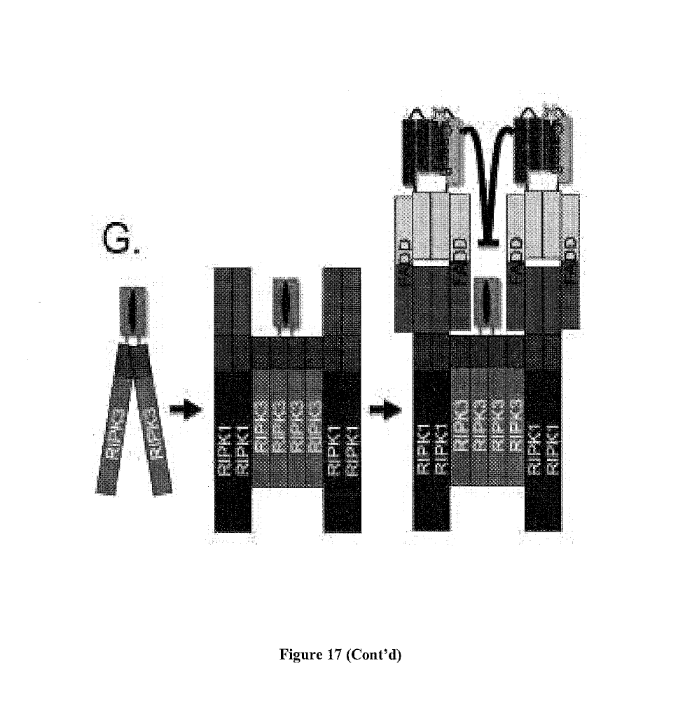

It is therefore likely that while forcing dimerization via the N-terminus of RIPK3 leads to proximity-induced autophosphorylation and MLKL binding, a lack of any structural constraint between the N-terminal kinase domain and the C-terminal RHIM means that C-terminal dimerization—as would occur with RHIM-RHIM interactions—does not result in proximity-induced autophosphorylation and MLKL binding. Taken together, these findings imply that RIPK3 autophosphorylation in the absence of oligomerization is sufficient to recruit and activate MLKL, but that RHIM-dependent oligomerization of RIPK3 is necessary to achieve sufficient kinase autoactivity to drive autophosphorylation during normal necrosome formation. See, FIGS. 17F and 17G.

When caspases are inhibited by pharmacological inhibitors, or under certain physiological conditions such as viral infections, RIPK1 and RIPK3 form the necrosome to initiate programmed necrosis (Cho et al., 2009; He et al., 2009; Zhang et al., 2009). Whereas it was originally thought to be associated with nonspecific cellular damages, genetic experiments in mice show that caspase-8-mediated cleavage and inactivation of RIPK1 and RIPK3 may be required for preventing extensive necrosis during embryonic development in order to ensure proper clonal expansion of lymphocytes and to prevent extensive necrosis and inflammation in skin and intestinal epithelium (Kaiser et al., 2011; Oberst et al., 2011; Welz et al., 2011; Zhang et al., 2011). In addition to caspase inhibition, assembly of the RIPK3/RIPK1 necrosome also involves intact RIPK1 and RIPK3 kinase activity (Cho et al., 2009). Both RIPK1 and RIPK3 contain Ser/Thr kinase domains (KDs) at their N-termini, and RIPK1 also has a death domain (DD) at its C terminus for recruitment to the TNF receptor signaling complex (Stanger et al., 1995; Sun et al., 1999; Yu et al., 1999).

Amyloids are fibrous protein aggregates composed of cross-13 structures and associated with many neurodegenerative (Chiti and Dobson, 2006) and infective prion diseases (Uptain and Lindquist, 2002). Amyloids can also perform normal cellular functions, such as host interaction, hazard protection, and memory storage (Chiti and Dobson, 2006). The discovery of cross-13 amyloid structures in protein complexation and signal transduction provides new insights into both the amyloid field and the signaling field. Unique segments of homologous sequences in RIPK1 and RIPK3 (RIP homotypic interaction motifs, RHIMs) mediate the assembly of heterodimeric filamentous amyloid structures (Sun et al., 2002) that activate RIPK3/RIPK1 kinase activity and serve as a functional signaling complex that mediates programmed necrosis (Cho et al., 2009).

The exact boundaries of RIPK RHIM domains are unclear, but the sequence conservation is centered around the I(V)QI(V)G motif. Whereas RIPK1 mutants flanking the RHIM domain does not show any defects in RIPK3 interaction and/or fibril formation, RIPK1 mutants near the center of the RHIM domain (i.e., for example, 1539D, I539P, Q540D, Q540P, I541D, I541P, G542D, G542P, A543D, Y544D, and/or N545D) result in RIPK3 interaction and/or fibril formation defects. In an inactive state, the RHIM domain may be hidden by long-range interactions within unstructured flanking sequences of RIPK1 and RIPK3, and/or possibly by RIPK1 ubiquitination. Kinase activation and resultant hyperphosphorylation may reduce this auto-inhibition, perhaps as a result of charge repulsion to expose the RHIM core, leading to enhanced complex formation. In turn, complex formation (i.e., for example, dimers and/or oligomers) further potentiates kinase activation through auto-phosphorylation and cross-phosphorylation, propagating the pronecrotic signal. Consequently, RHIM-mediated amyloidal RIPK3/RIPK1 fibrils may play a role in the activation of RIPK3/RIPK1 kinase activity and/or induction of programmed necrosis.

The role of the RHIM domain in activating RIPK3/RIPK1 kinase activity and mediating programmed necrosis may explain why full length RIPK3/RIPK1 heterodimers and full length RIPK3/RIPK3 are able to initiate necrosis while truncated RIPK3 homodimers are not. Dimerization (i.e., for example, RIPK3/RIPK1 heterodimers and/or RIPK3/RIPK3 homodimers) is thought to expose the RHIM domain to the cytosol, thereby permitting RHIM-dependent recruitment of more molecules of RIPK3 and/RIPK1, leading to the formation of oligomers (i.e. amyloids) and cell death.

Full-length RIPK1 and RIPK3 are present in normal cells and only induce death in response to the appropriate stimulus. In vivo, this stimulus is provided by the receptors that activate these proteins as described above. However, fusion proteins allow the formation of RIPK3/RIPK3 homodimers and RIPK3/RIPK1 heterodimers to be controlled by the introduction of a dimerizing agent (see below). Full length RIPK3/RIPK3 heterodimers and RIPK3/RIPK1 homodimers may form fusion proteins bearing a single Fv domain (i.e. RIPK3-Fv or RIPK1-Fv). The presence of an intact RHIM domain allows dimerization, that forms following addition of a dimerizing agent, to develop into an oligomer capable of inducing necrosis. However, modifications within the RHIM domain, such as a mutated RIPK3 (RIPK3^{ΔRHIM}) or a truncated RIPK3 (RIPK3^{ΔC}), are unable to recruit additional RIPK molecules and therefore require fusion proteins bearing multiple Fv domains (i.e. RIPK3^{ΔRHIM}-2xFv) to mediate oligomerization and necrosis. Although it is not necessary to understand the mechanism of an invention, it is believed that necrosis may be mediated by the formation of RIPK3 oligomers. It is further believed that while full length RIPK dimers can proceed to an oligomeric state on their own, truncated RIPK dimers may form oligomers upon the addition of Fv domains.

### II. Anti-Tumor Inflammatory Responses

Unlike cell death following apoptosis, necrosis results in the loss of cell membrane integrity and an uncontrolled release of products of cell death into the intracellular space. The rapid release of intracellular contents following cellular membrane damage is the cause of inflammation associated with necrosis (e.g., necroptosis). Externalization of phosphatidylserine (PS), the hallmark of apoptosis, is a very early feature of apoptotic death. In contrast, necrotizing cells are phagocytosed only after loss of membrane integrity by a macropinocytotic mechanism directed towards necrotic debris. This means that uptake is delayed and less efficient. The late uptake of necrotic cells allows the dying cells to activate pro-inflammatory and immune-stimulatory responses whereas apoptotic cell death is immunologically and inflammatorily silent.

Exposed or released intracellular components represent a potential source of autoantigens that might be processed and presented to initiate an autoimmune reaction. During cell death, several post-translational modifications occur, such as hyper-phosphorylation, (de)ubiquitination, methylation, citrullination, transglutaminase crosslinking and proteolytic cleavage. These modifications can increase the risk of an autoimmune response, especially when repeatedly presented to the immune system in a proinflammatory context. Spillage of the contents of necrotic cells into the surrounding tissue activates inflammatory signaling pathways. Depending on molecular signals from necrotic cells, diverse types of immune cells (neutrophils, macrophages, dendritic cells) become involved in the immune response. In contrast, apoptotic cells induce antigen presenting cells (APCs) to secrete cytokines that inhibit Th1 responses. Immature dendritic cells efficiently phagocytose a variety of apoptotic and necrotic tumor cells, but only the latter induce maturation and optimal presentation of tumor antigens. Besides the capacity of necrotically dying cells to induce an inflammatory response upon lysis and spillage of their contents, they can also actively release inflammatory cytokines due to the activation of NF-κB and MAPKs, a process that also involves RIPK1. of intracellular contents following cellular membrane damage is the cause of inflammation associated with necrosis

In one embodiment, the present invention contemplates a method for treating a tumor comprising inducing an RIPK-dependent necrosis, wherein tumor cell intracellular contents are released to create a potently immunogenic and inflammatory microenvironment, wherein an immune response is induced against the tumor cells. In one embodiment, the present invention contemplates inducing necrosis within (at least some of) the tumor cells within a patient such that an anti-tumor immune response is generated against the tumor. In one embodiment, the present invention contemplates that an anti-tumor immune response is generated in vitro (e.g. a tumor cell) and the resulting anti-tumor immune cells are introduced into a patient with a tumor such that the tumor is destroyed.

### III. Targeted Necroptic Genetic Engineering

In one embodiment, the present invention contemplates a method comprising transfecting a lymphocyte (e.g., a T-cell and/or a natural killer (NK) cell) comprising a chimeric antigen receptor with a vector comprising a nucleic acid sequence encoding a truncated RIPK3 fusion protein. In one embodiment, the expression of the vector induces necroptosis in a cell having an adverse reaction and/or uncontrolled growth.

A. Tumor-Specific T Cells

In one embodiment, the present invention contemplates methods of generating tumor-specific T cells using RIPK3/RIPK3 homodimers, RIPK3/RIPK1 heterodimers and/or truncated RIPK3 oligomers. Methods of generating tumor-specific T cells are known in the art, for example, immunotherapy with autologous tumor-reactive tumor infiltrating lymphocytes (TILs) immediately following a conditioning nonmyeloablative chemotherapy regimen have been shown to enhance clinical response rate in patients with metastatic melanoma; Journal of Immunotherapy, 28(1):53-62 (2005).

In one embodiment, the tumor-specific T cells are generated in vivo (i.e. within a tumor) by either 1) introducing the RIPK3/RIPK3 homodimer, RIPK3/RIPK1 heterodimer and/or truncated RIPK3 oligomer directly into the tumor (e.g. direct injection) or 2) introducing the nucleic acid molecules encoding the RIPK3/RIPK3 homodimer, RIPK3/RIPK1 heterodimer and/or truncated RIPK3 oligomer directly into the tumor (e.g. direct injection), such that RIPK proteins are expressed, followed by the introduction of the dimerizing agent such that dimers and/or oligomers are formed within the tumors. The necrosis that occurs in at least a portion of the cells of the tumor produce tumor-specific T cells that further attack and destroy the cells of the tumor.

In another embodiment, the tumor-specific T cells are generated in vitro (i.e. in cell culture, ex vivo) by either 1) introducing the RIPK3/RIPK3 homodimer, RIPK3/RIPK1 heterodimer and/or truncated RIPK3 oligomer directly into those cultured cells (e.g., for example, by electroporation) or 2) introducing the nucleic acid molecules encoding the RIPK3/RIPK3 homodimer, RIPK3/RIPK1 heterodimer and/or truncated RIPK3 oligomer into those cultured cells under conditions such that their respective proteins are expressed (e.g. electroporation or transformation with plasmids bearing heterodimer or homodimer fusion proteins as described below). Introduction of a dimerizing agent then facilitates necrosis in at least a portion of those cells. The breakdown products of necrotic cells may then be used to stimulate the generation of tumor-specific T cells either in vitro or in vivo using methods known in the art. For example, immune cell cultures may be exposed to the cellular breakdown products in vitro such that tumor-specific T cells are generated followed by the introduction of those T cells into a patient with a tumor. Alternatively, the cellular breakdown products may be administered directly to a patient such that tumor-specific T cells are generated within the patient.

B. A Suicide Gene Encoding a Truncated RIPK3 Fusion Protein

T lymphocytes (T cells) expressing a chimeric antigen receptor (CAR) can be adoptively transferred to target a range of human malignancies, including non-Hodgkin's and Hodgkin's lymphomas. CARs most commonly combine the antigen-binding specificity of a monoclonal antibody with an effector endodomain of a CD3/T-cell receptor complex (Z-chain), and redirects the specificity of T lymphocytes toward surface antigens expressed by tumor cells. CARs that target B-lineage-restricted antigens such as CD19, CD209 and the light chain of human immunoglobulins, or CD30 expressed by Reed-Sternberg cells, have been cloned and validated in preclinical lymphoma/leukemia models, and some are currently in phase I clinical trials. However, it is evident from both clinical trials and preclinical models that the expansion and persistence of CAR-modified T cells in vivo are hampered by the lack of costimulatory signals after engagement with target antigens, as many tumor cells do Wn-regulate their expression of the costimulatory molecules required for optimal and sustained T-cell function, proliferation and persistence.

In one embodiment, the present invention contemplates an isolated mammalian cell comprising: (a) a chimeric antigen receptor (CAR) that targets an necrosis antigen; (b) ectopic expression of a necrosis antigen (b) a suicide gene; and (c) a detectable gene product. In certain aspects, the chimeric antigen receptor further comprises a costimulatory endodomain, such as a CD28 costimulatory endodomain, a 4-IBB costimulatory endodomain, an OX40 costimulatory endodomain, or a combination thereof. In particular embodiments of the invention, the cell comprises a polynucleotide that expresses the chimeric antigen receptor, a polynucleotide that expresses the necrosis antigen, a polynucleotide that expresses a suicide gene, and/or a polynucleotide that expresses CAR, the necrosis antigen and the suicide gene. In certain embodiments, the suicide gene comprises a truncated RIPK3 gene. In one embodiment, the truncated RIPK3 gene is a truncated RIPK3 fusion gene. In one embodiment, the suicide gene comprises an RIPK1 gene. In one embodiment, the mammalian cell includes, but is not limited to, a T lymphocyte, a natural killer cell, a lymphokine-activated killer cell, and/or a tumor infiltrating lymphocyte.

In particular embodiments of the invention, CAR, a necrosis antigen gene, suicide gene, or a combination thereof are housed on a vector, such as a plasmid or viral vector, including a retroviral vector, adenoviral vector, adeno-associated viral vector, or lentiviral vector. In a vector nucleic acid construct employed in the present invention, a promoter, such as the LTR promoter of the ret roviral vector, is operably linked to a nucleic acid sequence encoding the particular moieties of the vector, including the chimeric antigen receptor of the present invention, the necrosis antigen and/or the suicide gene, i.e., they are positioned so as to promote transcription of the messenger RNA from the DNA encoding the gene product.

The LTR promoter can be substituted by a variety of promoters for use in T cells that are well-known in the art (e.g., the CD4 promoter disclosed by Marodon, et al. (2003) Blood 101 (9):341 6-23). The promoter can be constitutive or inducible, where induction is associated with the specific cell type or a specific level of maturation, for example. Alternatively, a number of well-known viral promoters are also suitable. Promoters of interest include the β-actin promoter, SV40 early and late promoters, immunoglobulin promoter, human cytomegalovirus promoter, and the Friend spleen focus-forming virus promoter. The promoters may or may not be associated with enhancers, wherein the enhancers may be naturally associated with the particular promoter or associated with a different promoter.

In one embodiment, the present invention contemplates a method in which engineered CAR-modifed T cells receive stimulation from expressed necrosis antigens expressed within the T cell and/or incorporated be release from nearby necrotic cells. Embodiments also include a suicide gene that can be pharmacologically activated to eliminate cells comprising the necrosis antigens.

In embodiments of the invention, there are at least nucleic acids, polypeptides, vectors, and/or cells that concern recombinantly engineered compositions having at least an inducible suicide gene and a necrosis antigen. In addition, a chimeric antigen receptor (CAR) and/or a detectable gene product may be included in the composition. In some embodiments that include a vector, the CAR may be provided on a vector separate from a vector that harbors the inducible suicide gene and the necrosis antigen. In embodiments for the detectable gene product, the cells that harbor the polynucleotide that encodes the detectable gene product are identifiable, such as by standard means in the art, including flow cytometry, spectrophotometry, or fluorescence, for example.

In some embodiments of the invention polynucleotides harboring the cytokine, inducible gene product, and CAR and/or detectable gene product are integrated into the genome of a mammalian cell, although in some embodiments of the invention the polynucleotides are not integrated into the genome.

In some embodiments of the invention, the chimeric antigen receptor (CAR) comprises a fusion of single-chain variable fragments (scFv). In one embodiment, the CAR may comprise a CD28 costimulatory endodomain and a CD3-Zeta endodomain. The exodomain of the CAR may be considered an antigen recognition region and can be anything that binds a given target antigen with high affinity. The CAR may be of any kind, but in specific embodiments the CAR targets necrosis antigens. For example, the CARs may target any type of necrosis antigen derived from a tumor cell including, but not limited to B-cell-derived malignancies, such as lymphoma and leukemia, lung cancer, liver cancer, prostate cancer, pancreatic cancer, colon cancer, skin cancer, ovarian cancer, breast cancer, brain cancer, stomach cancer, kidney cancer, spleen cancer, thyroid cancer, cervical cancer, testicular cancer, and/or esophageal cancer

In one embodiment, the present invention contemplates a CAR comprising an intracellular receptor signaling domain including, but not limited to, a Zeta chain of CD3, an Fey RIII costimulatory signaling domain, CD28, DAP10, CD2, alone or in combination with CD3/Zeta, for example. In other embodiments, the intracellular domain (which may be referred to as the cytoplasmic domain) comprises part or all of one or more of domains including, but not limited to a TCR Zeta chain, CD28, OX40/CD134, 4-1BB/CD137, FceRIy, ICOS/CD278, ILRB/CD122, IL-2RG/CD132, and/or CD40. One or multiple cytoplasmic domains may be employed, as so-called third generation CARs have at least 2 or 3 signaling domains fused together for additive or synergistic effect, for example.

### IV. RIPK3/RIPK1 Dimerization

Methods for producing fusion proteins, including for example RIPK3 and/or RIPK1 homodimers, heterodimers and oligomers are known in the art (e.g., ARGENT™ Regulated Homodimerization Kit and ARGENT™ Regulated Heterodimerization Kit; Ariad Pharmaceuticals, Cambridge Mass.). Many cellular processes are triggered by the induced interaction, or “dimerization”, of signaling proteins. Examples include the clustering of cell surface receptors by extracellular growth factors, and the subsequent stepwise recruitment and activation of intracellular signaling proteins. A chemical inducer of dimerization, or “dimerizer”, is a cell-permeable organic molecule with two separate motifs that each bind with high affinity to a specific protein module. Any cellular process activated by protein-protein interactions can in principle be brought under dimerizer control by fusing the protein(s) of interest to the binding protein recognized by the dimerizer. Addition of the dimerizer then crosslinks the chimeric signaling protein thereby activating the cellular event that it controls. There are two classes of dimerizers: homodimerizers and heterodimerizers. Homodimerizers incorporate two identical binding motifs, and can therefore be used to induce self-association of a single signaling domain. Heterodimerizers have two different binding motifs, allowing the dimerization of two different signaling domains when fused to the two appropriate ligand binding domains.

Induced protein homodimerization is broadly applicable, and in addition to the methods described herein a large number of signaling proteins have been brought under homodimerizer control. Examples include, but are not limited to, transmembrane signaling receptors (such as Fas, gp130, and the receptors for Epo, Tpo, insulin, TGF-β PDGF, EGF and HGF); intracellular signaling molecules (such as Src, Sos, Vav, ZAP70, Raf, Bax, FADD, CED3, caspases 1, 3, 8 and 9, RIF′, IKKε, and T cell receptor zeta chain); and other cellular proteins with dimerization-based mechanisms, including integrins, cadherins and transcription factors.

Regulated dimerization has applications in many areas of functional genomics research and drug discovery. Inducible alleles of orphan receptors or other signaling proteins can be created with no knowledge of the natural ligand. These systems can be used for functional analysis of the signaling pathway in multiple cell types, potentially identifying downstream target proteins, or genes whose expression is modulated by the signaling event. Inducible animal models can be established of disease states associated with an activated signaling protein. In addition, cell lines in which a specific signal can be chemically induced may be useful in the configuration of targeted cell-based assays for small molecule drugs.

The reagents in the ARGENT Regulated Homodimerization Kit are based on the human protein FKBP12 (FKBP, for FK506 binding protein) and its small molecule ligands. FKBP is an abundant cytoplasmic protein that serves as the initial intracellular target for the natural product immunosuppressive drugs FK506 and rapamycin. Conventionally, a dimerizer was created by chemically linking two molecules of FK506 in a manner that eliminated immunosuppressive activity. The resulting molecule, called FK1012, was able to crosslink fusion proteins containing wild type FKBP domains. A second generation FKBP homodimerizer, AP1510, was subsequently developed that has the advantages of being completely synthetic, as well as being smaller and simpler than FK1012 and more potent in many applications.

The affinity and specificity of these molecules was further improved by eliminating their ability to bind to endogenous FKBP. These homodimerizers, AP1903 and AP20187, bind with subnanomolar affinity to FKBPs with a single amino acid substitution, Phe36Val (Fv), while binding with 1000-fold lower affinity to the wild type protein. The new system invariably provides more potent activation of homodimerization, and have pharmacologic properties suitable for in vivo use. AP20187 and Fv form the basis of the reagents provided in the ARGENT Regulated Homodimerization Kit (herein incorporated by reference).

It is also possible to use commercially available kits to induce heterodimerization by fusing two different signaling proteins to the same ligand binding domain, wherein the addition of the homodimerizer creates a mixture of homodimeric and heterodimeric complexes (e.g., ARGENT™ Regulated Heterodimerization Kit).

To control the activity of a signaling domain, the domain of interest is fused to one or more copies of an Fv domain and the dimerization state is controlled by administration of the dimerizer. The ARGENT™ Regulated Homodimerization Kit contains two plasmids, pC₄-Fv1E and pC₄M-Fv2E, and an aliquot of dimerizer (AP20187). In pC₄-Fv1E, a chimeric fusion protein containing a single copy of Fv (Fv1) followed by a carboxy-terminal epitope tag (E, from the influenza hemagglutinin [HA] gene) is expressed under control of the human CMV enhancer/promoter. The Fv domain is flanked by XbaI and SpeI sites. To fuse the protein of interest to a single Fv domain it is cloned into the adjacent XbaI or SpeI sites. Unless the domain fused to FKBP contains a signal that targets it to another location, fusion proteins should be localized to the cytoplasm by default as there is no targeting signal in this vector (the amino terminus of this fusion protein, upstream of the XbaI site, consists only of a methionine). In pC₄M-Fv2E, a chimeric fusion protein containing an amino-terminal myristoylation signal (M), two copies of Fv (Fv2), followed by a carboxy-terminal epitope tag (E, from the influenza hemagglutinin [HA] gene) is expressed under control of the human CMV enhancer/promoter. The two Fv domains are flanked by XbaI and SpeI sites. To fuse the protein of interest to two Fv domains it is cloned into the adjacent XbaI or SpeI sites. One of the Fv domains has changes in the codons used that do not change the amino acid sequence, but which significantly reduce the match between the Fv domains at the nucleotide level.

The creation of fusion proteins is based on a standard cloning strategy involving the stepwise addition of compatible XbaI-SpeI fragments. To do this, amplify the coding sequence of interest by PCR so that it contains the six nucleotides specifying an XbaI site immediately 5′ to the first codon (take care not to create an overlapping Dam methylation sequence, GATC, on either strand), and the six nucleotides specifying a SpeI site immediately 3′ to the last codon. Then, for example, to fuse the protein of interest amino terminal to two Fv domains, clone the XbaI-SpeI fragment into the XbaI site of pC₄M-Fv2E (XbaI and SpeI have compatible cohesive ends). If inserted in the proper orientation, the XbaI and SpeI sites, now flanking the new fusion protein, will be maintained, with the junction of the two peptides consisting of the two amino acids specified by the SpeI and XbaI sites that were fused. Alternatively, to fuse the XbaI-SpeI fragment carboxy-terminal to two Fv domains, insert it into the SpeI site of pC4M-Fv2E. In both cases, since the flanking XbaI and SpeI sites are maintained, additional fragments can still be fused at the amino- and carboxy-terminal ends if desired. This strategy can also be applied to create three tandem Fv domains. For example, the XbaI-SpeI fragment of pC4-Fv1E can be inserted into the SpeI site of pC₄M-Fv 2E (or vice versa).

The number of Fv domains bested suited for each application varies. Fusion to a single Fv domain is generally preferred if formation of dimers is sufficient to induce the desired signaling event. Fusion to two or more Fv domains may be preferred when induction of a signaling event requires the formation of higher order oligomers.

Commercially available dimerization kits can be used to control any signaling process that involves regulated protein-protein interactions, in particular, for creating specific interactions between two different proteins, especially where the directionality of dimerization is desired to be controlled. One dimerizer, AP21967, is suitable for in vivo use and has been used successfully in mice. Other reagents are based on the human protein FKBP12 (FKBP, for FK506 binding protein) and its small molecule ligands. FKBP is an abundant cytoplasmic protein that serves as the initial intracellular target for the natural product immunosuppressive drugs FK506 and rapamycin. Both these drugs naturally act as heterodimerizers, and both have been used as the basis for heterodimerization systems, as has FK-CsA, a cyclosporin-FK506 hybrid molecule. Rapamycin functions by binding with high affinity to FKBP, and then to the large PI3K homolog FRAP, thereby acting as a heterodimerizer to join the two proteins together. To use rapamycin to induce heterodimers between proteins of interest, one of the proteins is fused to FKBP domains, and the other to a 93 amino acid portion of FRAP, termed FRB, that is sufficient for binding the FKBP-rapamycin complex.

In some cases, the use of rapamycin as a heterodimerizing reagent may be compromised by its cell cycle inhibitory effects (the result of inhibiting FRAP kinase activity, which in T cells leads to immunosuppression). This limitation has been addressed by engineering the system to function with non-immunosuppressive analogs of rapamycin, herein referred to as rapalogs. These compounds have been chemically modified so that they no longer can bind to wild type endogenous FRAP, greatly reducing immunosuppressive activity. The compounds can however bind to a modified FRAP that contains a single designed amino acid change (T2098L). Incorporation of this mutation into the FRB domain used to make protein fusions allows a rapalog to be used to specifically heterodimerize engineered proteins without interfering with the activity of endogenous FRAP.

This rapamycin system forms the basis of the ARGENT Heterodimerization Kit, which provides constructs containing FKBP and the mutant FRB, and a non-immunosuppressive rapalog called AP21967. These and related reagents have been used to control the localization and activity of signaling domains as described above. Other, redesigned systems, retain the ability to respond to rapamycin itself, as well as AP21967. Therefore experiments can be carried out with either ligand, as appropriate.

To control the activity or localization of signaling domains, one of the domains of interest is fused to one or more copies of an FKBP domain and the other to a mutant FRB domain. This allows the dimerization state to be controlled by administration of the rapalog AP21967. Further, dimerization kits may also contain plasmids (e.g., for example, pC₄EN-F1, pC₄M-F2E, pC₄-RHE) where these plasmids provide an assortment of components (i.e. mutant FRB domain, multiple FKBP domains, an epitope tag and localization sequences) that can be manipulated to generate protein fusions whose activity and localization can be controlled by dimerizer.

The pC₄EN-F1 expression plasmid includes a chimeric fusion protein containing an amino terminal epitope tag (E, from the influenza hemagglutinin [HA] gene) and nuclear localization signal (from SV40 large T antigen), followed by a single copy of FKBP12, expressed under control of the human CMV enhancer/promoter. The FKBP domain is flanked by XbaI and SpeI sites. To fuse the protein of interest to a single FKBP domain the nucleic acid sequence encoding the protein of interest is cloned into the adjacent XbaI or SpeI sites as described below.

The pC₄M-F2E expression plasmid includes a chimeric fusion protein containing an amino-terminal myristoylation signal, two copies of FKBP, followed by a carboxy-terminal epitope tag (from the influenza hemagglutinin [HA] gene) expressed under control of the human CMV enhancer/promoter. The two FKBP domains are flanked by XbaI and SpeI sites. To fuse the protein of interest to two FKBP domains the nucleic acid sequence encoding the protein of interest is cloned into the adjacent XbaI or SpeI sites as described below. One of the FKBP domains has changes in the codons used that do not change the amino acid sequence, but which dramatically reduce the match between the FKBP domains at the nucleotide level.

The pC₄-R_{H}E expression plasmid includes a chimeric fusion protein containing a single copy of the modified FRB, followed by a carboxy-terminal epitope tag (from the influenza hemagglutinin [HA] gene) expressed under control of the human CMV enhancer/promoter. R_{H} consists of amino acids 2021-2113 of human FRAP in which the threonine at amino acid 2098 was mutated to leucine, to accommodate the chemical substitution that blocks AP21967 binding to wild type FRAP. The R_{H} domain is flanked by XbaI and SpeI sites. To fuse the protein of interest to a single R_{H}domain the nucleic acid sequence encoding the protein of interest is cloned into the adjacent XbaI or SpeI sites as described below.

The creation of fusion proteins is based on a standard cloning strategy involving the stepwise addition of compatible XbaI-SpeI fragments. This is achieved by first amplifying the coding sequence of interest by PCR so that it contains the six nucleotides specifying an XbaI site immediately 5′ to the first codon (take care not to create an overlapping Dam methylation sequence, GATC, on either strand), and the six nucleotides specifying a SpeI site immediately 3′ to the last codon. Then, for example, fusing the amino terminal end of the protein of interest to two FKBPs, and then cloning the XbaI-SpeI fragment into the XbaI site of pC₄M-F2E (XbaI and SpeI have compatible cohesive ends). If inserted in the proper orientation, the XbaI and SpeI sites, now flanking the new fusion protein, will be maintained, with the junction of the two peptides consisting of the two amino acids specified by the SpeI and XbaI sites that were fused. To fuse the XbaI-SpeI fragment carboxy-terminal end to two FKBPs, insert it into the SpeI site of pC₄M-F2E. In both cases, since the flanking XbaI and SpeI sites are maintained, additional fragments can still be fused at the amino- and carboxy-terminal ends if desired. If the sequence to be fused contains internal XbaI or SpeI sites, fusions can still be made either by using XbaI or SpeI at both ends, or by using NheI or AvrII which also generate ends that are compatible with. XbaI and SpeI. The sequence between the SpeI and BamHI sites of pC₄EN-F1, pC₄M-F2E and pC₄-R_{H}E contains an in-frame stop codon (in some cases proceeded by an HA epitope tag). Therefore, stop codons should not be included in the fused sequences. The number of FKBP and FRB domains bested suited for each application may vary. While fusing one FKBP domain and one FRB domain to each signaling protein may work well for some applications, the use of two FKBP domains may be preferable for other applications.

In some embodiments, the present invention contemplates the use of dimerizing agents such as rapamycin or derivatives thereof. AP21967 is a chemically modified derivative of rapamycin that can be used to induce heterodimerization of FKBP and FRB T2098L-containing fusion proteins. AP21967 is greater than 1000-fold less immunosuppressive than rapamycin as measured in an in vitro splenocyte proliferation assay. In some studies to date, AP21967 is non-toxic to cells at up to 1 μM concentrations, or mice at up to 30 mg/kg doses. AP21967 cannot be used to heterodimerize proteins containing a wild type FRB domain. Note, however, that the presence of the T2098L mutation in FRB has little or no detrimental effect on the binding of rapamycin. Therefore, as noted earlier, rapamycin can also be used to dimerize fusion proteins made using the reagents in this kit. Working concentrations of AP21967 can be obtained by adding compound directly from ethanol stocks, or by diluting serially in culture medium just before use. In the latter case we recommend that the highest concentration does not exceed 5 μM, to ensure complete solubility in the (aqueous) medium. In either case, the final concentration of ethanol in the medium added to mammalian cells should be kept below 0.5% (a 200-fold dilution of a 100% ethanol solution) to prevent detrimental effects of the solvent on the cells.

Rapamycin is available commercially from Sigma (cat #R0395) or Affinity BioReagents (cat # IR-022). Similarly, AP20187 is a synthetic dimerizer that can be used to induce homodimerization of Fv domain-containing fusion proteins. AP20187 has no immunosuppressive activity and is non-toxic to cells. AP20187 cannot be used to dimerize wild type FKBP domains. AP20187 has been successfully used in mice with maximal effects seen at doses in the range of 0.5-10 mg/kg delivered intravenously. The AP20187-based system has the advantages of working at lower concentrations, and AP20187 has better pharmacokinetic properties than AP1510, allowing it to be used in vivo.

### V. Caspases and Necrosis

While the role of caspases in apoptosis is well established, little is known about the role of these proteases in the process of programmed necrosis. The present application is based on the surprising finding that embryonic lethality as a result of ablation of caspase-8 or its adapter protein, FADD, is fully rescued by deletion of RIPK3, a kinase required for programmed necrosis. The present studies indicate that a complex of FADD, caspase-8, and FLIP (a caspase-like molecule that lacks a catalytic cysteine) protects against RIPK-dependent necrosis. This is further supported by findings that the FADD-FLIP-RIPK3 TKO mouse develops normally.

The following studies are designed to delineate the functions of these proteins in development and cancer:

1) Identifying the developmental target protected by the FADD-caspase-8-FLIP complex.

The phenotypes of caspase-8, FADD, and/or FLIPL knockouts all show embryonic lethality around e10.5, associated with a defect in yolk sac vascularization. In one embodiment, the studies presented herein are designed to verify that early progenitors of vascular endothelium and hematopoietic cells serve as the earliest and most important targets of this developmental defect. In so doing, additional targets of RIPK3-necrosis are identified and the signaling pathways engaged in such embryonic lethality are elucidated.

2) Regulation of RIPK-dependent necrosis in oncogenesis.

Caspase-8, which in humans is present on chromosome 2q33, is often silenced or deleted in human neuroblastoma, small cell lung carcinoma, and other cancers. This represents a paradox, however, as such loss in many cell types sensitizes cells to RIPK-dependent necrosis. In one embodiment, the studies presented herein explore how the loss of caspase-8 can fail to sensitize tumor lines to RIPK-dependent necrosis. These studies include how RIPK3 transcription is controlled in primary and transformed tissues and the role of RIPK1 and the tumor suppressor, CYLD, in controlling RIPK-dependent necrosis.

3) RIPK-dependent necrosis as an avenue for therapy.

Many approaches to cancer therapy seek to promote apoptosis, which may or may not promote ancillary anti-tumor immunity. Shifting signals to RIPK-necrosis may: a) prevent iatrogenic damage in tissues resistant to this forth of death (e.g., liver); while b) promoting an inflammatory mode of tumor cell death. “Pure” RIPK3-induced necrosis versus apoptosis is modeled to examine the anti-tumor consequences and to explore a counterintuitive approach to triggering RIPK-dependent necrosis in autochthonous and grafted tumors by death receptor ligation in vivo. The possibility that tumor neo-vasculature is targeted is also explored. The studies detailed herein represent the potential to greatly increase our understanding of the fundamental processes controlling cell life and death, both in normal development and in cancer.

A. RIPK-Dependent Necrosis in Development and Cancer

The elucidation of the core apoptotic pathways in animals was a major achievement of the 1990's. Towards the end of that decade the effects of genetic ablation in mice of many of the components of these pathways were elucidated, which in most cases resulted in developmental effects consistent with their roles in cell death, i.e., in the appearance of extra cells. These included deletion of an executioner caspase, caspase-3, components of the mitochondrial pathway (Bim, Bax and Bak, APAF1, caspase-9), and a death receptor, CD95 or its ligand. In striking contrast, deletion of either of two elements promoting apoptosis in the death receptor pathway, caspase-8 or its adapter, FADD, produced a different effect: embryonic lethality that could not be ascribed to a failure in apoptosis. Recently, it has been shown that this lethality is rescued upon ablation of RIPK1 or RIPK3, two kinases involved in a process of programmed necrosis. These findings along with additional studies led to the central hypothesis that a proteolytically active complex of FADD, caspase-8, and the caspase-8-like molecule, FLIP, inhibits RIPK-dependent necrosis without inducing apoptosis. A corollary is that the essential functions of these proteins in development reside in the control of apoptotic and necrotic cell death mediated by caspase-8 and RIPK3, respectively. If those proteins perform other indispensable non-apoptotic functions they must depend on RIPK3 as well.

This hypothesis led to an exploration of the regulation of RIPK3-dependent necrosis in developing and adult animals, thereby defining tissue types sensitive to this form of cell death. Since evasion of cell death is a hallmark of cancer, one embodiment of the present invention contemplates that such regulation is likely to play a role in the control of oncogenesis. A further embodiment contemplates that this role in the control of oncogenesis may be exploited therapeutically. The experimental strategies outlined below follow directly from these considerations.

Most physiological, and many pathological, cell deaths in the body proceed by apoptosis; an ordered process orchestrated by the caspase proteases (1). This involves the formation of “caspase activation platforms” involving adapter proteins that bind and thereby activate initiator caspases (such as caspases-8 and -9). These adapter proteins cleave and thereby activate executioner caspases (e.g., caspases-3 and -7) that in turn cleave many substrates to cause apoptosis. The delineation of the apoptotic pathways that coordinate caspase activation has provided fundamental insights into development, homeostasis, immune function, cancer and aging. The main pathways of apoptosis are: a) the mitochondrial pathway, in which BCL-2 proteins control mitochondrial permeabilization which results in caspase activation, and b) the death receptor pathway, in which an adapter protein, FADD, binds to and activates caspase-8 to precipitate apoptosis.

Germ line or conditional deletion of components of the mitochondrial pathway of apoptosis generally produces phenotypes consistent with the roles of these proteins in controlling cell death. In contrast, deletion of either the FADD adapter or caspase-8 (elements of the death receptor pathway) results in embryonic lethality around e10.5, a phenotype that cannot be ascribed to a failure to engage apoptosis (2, 3). Similarly, deletion or inhibition of caspase-8 or FADD in T lymphocytes does not block proliferation upon activation (4-6). Knockdown of caspase-8 affects differentiation of human villous trophoblast in vitro (7, 8). Such observations have contributed to extensive literature describing non-apoptotic effects of FADD and caspase-8 in cell adhesion, motility and migration, cell cycle progression, NF-kB activation and suppression of inflammation (9).

Meanwhile, in some cells it has been noted that engagement of death receptors in the presence of caspase inhibitions, rather than protecting the cells, promotes a necrotic cell death (10). This cell death depends on two kinases, RIPK1 (10-12) and RIPK3 (13-15), and can be blocked by the RIPK1 inhibitor, necrostatin-1 (nec1) (12). In particular, it is caspase-8 that antagonizes this RIPK-dependent necrosis (16, 17). Strikingly, it has been reported that the embryonic lethality of the caspase-8 null mouse is fully rescued by ablation of RIPK3 (18, 19). Similarly, the development of FADD KO mice is partially rescued by ablation of RIPK1 (20); although these animals die peri-natally (a phenotype of the RIPK1 KO (21), which might obscure other roles for FADD). The caspase-8 RIPK3 DKO mice are developmentally normal but over time develop a severe lympho-accumulative disorder resembling that of mice or humans lacking CD95 or its ligand (18, 19, 22). This is consistent with a loss of death receptor mediated apoptosis in these DKO animals. These findings along with additional results provided below outline a model that accounts for the regulation of RIPK1-RIPK3 function in development. The complexities of these signaling pathways have been reviewed in detail by the inventors and others (9, 23, 24).

Understanding the interplay of RIPK1-RIPK3 and FADD-caspase-8 (as well as FLIP) has implications for elucidating the control of cell death in development and homeostasis. Further, the role of RIPK3 in the response to viral infection (15, 25) and the function of viral inhibitors of this interaction (25) underscore the importance of these studies. Finally, delineation of the regulator interactions controlling RIPK-dependent necrosis will impact the design of strategies to confront cancer and inflammatory disease.

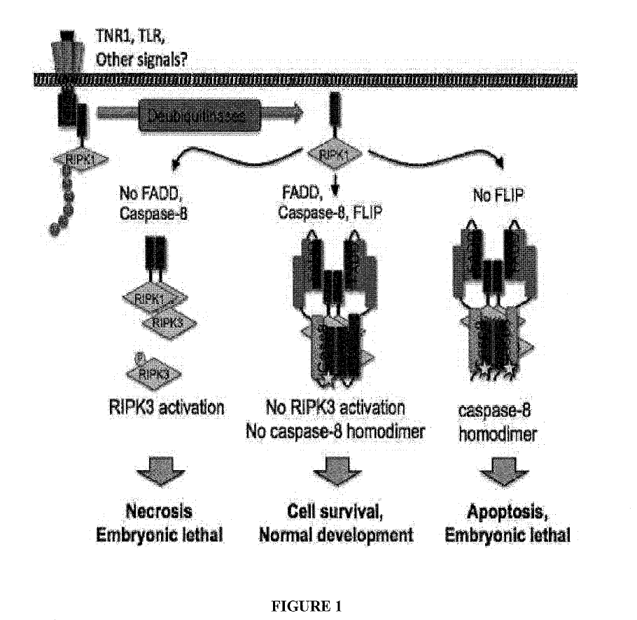

Previous work strongly suggests that a complex of FADD, caspase-8, and the caspase-like protein, FLIP (which lacks a catalytic cysteine), constitutes a catalytically active entity that does not promote apoptosis but antagonizes necrosis induced by RIPK1-RIPK3 interactions. (18, 26-28) Recent findings have led to a model as outlined in FIG. 1, wherein attached death receptors or other signals (e.g. TLR engagement of TRIF (26) promote the deubiquitination of RIPK1 (e.g. by CYLD (26, 30)) which recruits both RIPK3 and FADD. FADD recruits caspase-8 and FLIP, and the latter proteolytic heterodimer antagonizes RIPK3 activation. As a consequence, cells survive only if FADD, caspase-8, and FLIP are all present (FIG. 1).

At least four lines of evidence support a model of caspase-8-FLIP function in blocking RIPK-dependent necrosis where:
1) Caspase-8 that cannot be cleaved between the large and small subunits does not form stable homodimers and cannot effect apoptosis (27), but is proteolytically active when heterodimerized with FLIP (18, 28), yet transgenic expression of such a non-cleavable caspase-8 rescues development of the caspase-8 KO without permitting caspase-8-mediated apoptosis (29);
2) The pox virus serpin CrmA is a potent inhibitor of caspase-8 homodimers but does not efficiently block the catalytic activity of caspase-8-FLIP, whereas expression of CrmA in cells can block caspase-8 dependent apoptosis without sensitizing those cells for RIPK-dependent necrosis in response to TNF (18);
3) Cells expressing BCL-xL (a member of the Bcl-2 family of proteins) that are protected from TNF-induced apoptosis activate caspase-8 in the absence of FLIP, but this does not prevent RIPK-dependent necrosis (which does not occur if FLIP expression is sustained (18); and
4) Knockout of any of the three components of the proposed protective complex (FADD, caspase-8, or FLIP) produces the same phenotype (i.e. embryonic lethality at e10.5) with a failure in yolk sac vasculature formation (3, 30-32).
   Therefore, while earlier studies showed that FLIP blocks caspase-8-mediated apoptosis, it is now proposed that the resulting FADD-caspase-8-FLIP complex blocks RIPK1-RIPK3 signaling for necrosis. This also explains how caspase-8 can function to prevent necrosis without itself inducing apoptosis. It is not caspase-8 per se, but the catalytic activity of caspase-8-FLIP complex that functions in this survival role. This is further supported by the recent findings described herein.

B. FADD and FLIP and Developmental Function

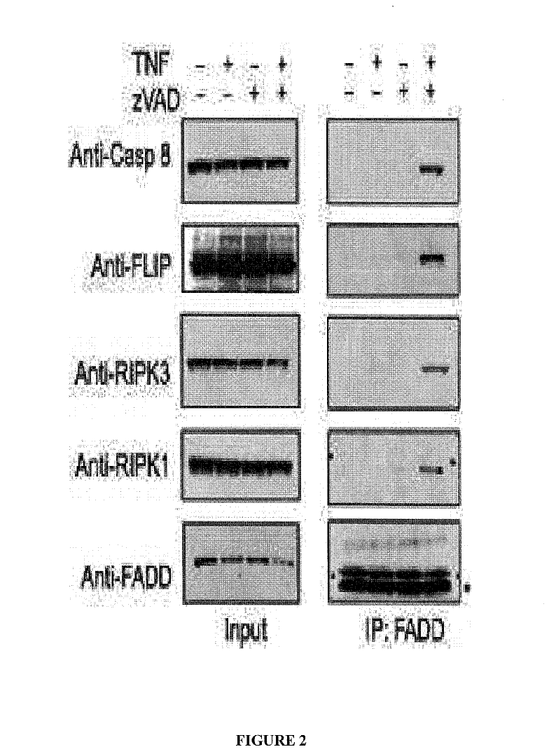

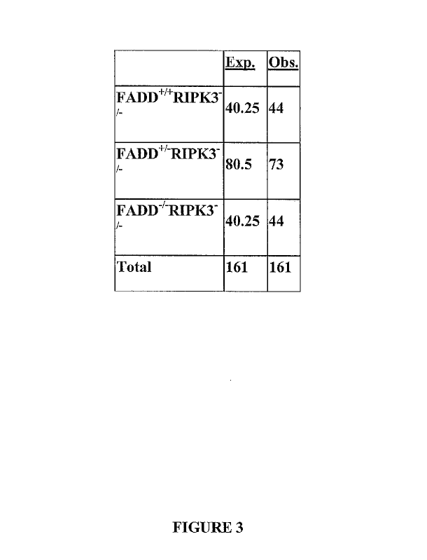

The data presented herein indicate that upon TNFR1 ligation in vitro, a complex comprising FADD, caspase-8, FLIP and RIPK1 induces RIPK3 formation, but that the caspase activity (blocked by zVAD-fmk) disrupts this complex (FIG. 2). It is proposed that the protective effect of caspase-8 in development is in the form of a FADD-caspase-8-FLIP complex that prevents RIPK-dependent necrosis but does not promote apoptosis. Further genetic analysis confirms this scenario, as follows.

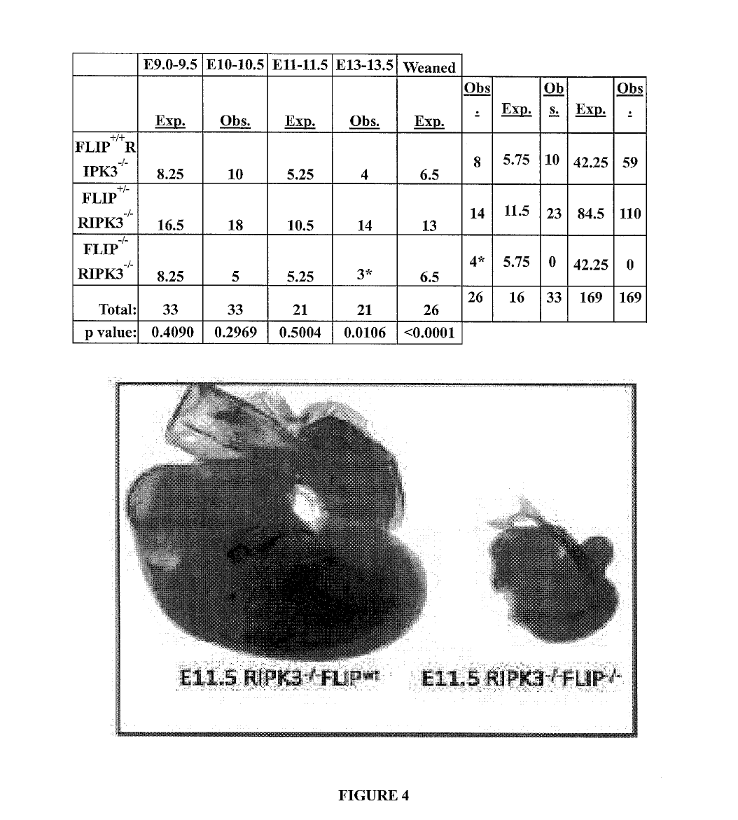

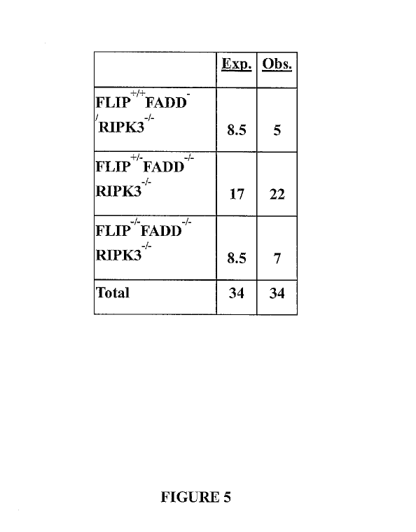

Like caspase-8, the knockout of FADD is lethal (3, 4), but the present results demonstrate that it is rescued by ablation of RIPK3 (FIG. 3). In contrast, deletion of FLIP, also lethal, is not rescued by ablation of RIPK3 and lethality occurs around e10.5 (FIG. 4). One embodiment contemplates that this is because in the absence of FLIP, FADD-caspase-8 promotes unconstrained apoptosis. To test this, FADD KO mice (lethal) were crossed to FLIP-RIPK3 DKO mice (lethal). Surprisingly, the resulting triple KO (TKO) mice are developmentally normal (FIG. 5). This result represents a situation in which the cross of two lethal KO genotypes (e.g., FADD KO and/or FLIP-RIPK3 DKO) produces a viable TKO mouse. This surprising result provides strong support for a model as outlined above (FIG. 1) and is formal evidence that embryonic survival depends on a complex of FADD, caspase-8, and FLIP that prevents RIPK3-dependent embryonic lethality. It should be noted that the combined KO of both caspase-8 and FLIP, which are separated by only 30 kB, cannot be performed by cross-breeding.

C. RIPK-Necrosis Induction Systems

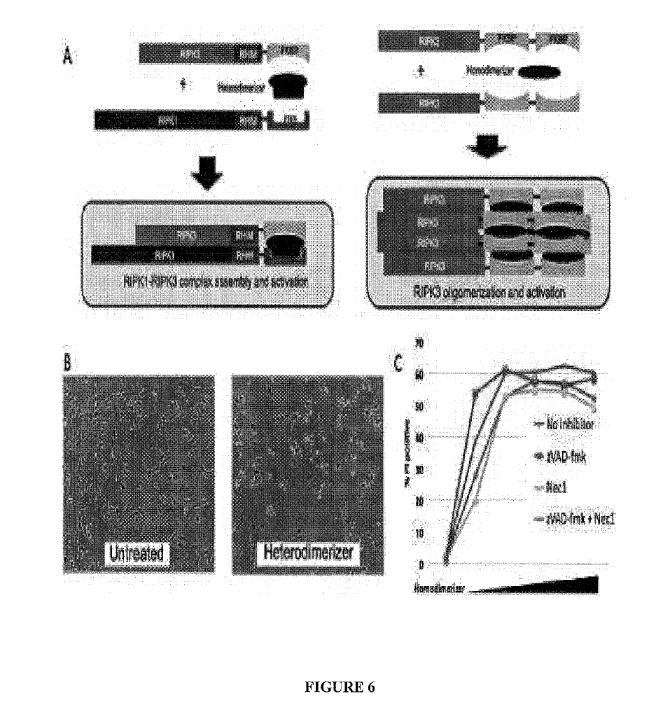

An inducible dimer system has been employed to trigger homodimerization of caspase-8 in cells (27). The data provided herein support methods to induce RIPK-dependent necrosis by related approaches (FIG. 6A). Heterodimerization of RIPK1 and RIPK3 results in necrosis (see FIG. 6B) which is enhanced by inhibition of caspases and blocked by inhibition of RIPK1 (with nec1, data not shown). In contrast, truncated RIPK3 (lacking the RHIM domain for interaction with RIPK1) can only be oligomerized when FKBP domains (e.g., for example, two domains) that allow multiple monomers to be brought together by the dimerizing agent. Addition of the homodimerization agent to cells expressing this construct induces extremely rapid necrosis that that depends on the kinase activity of RIPK3, but is not affected by inhibition of caspases or nec1 treatment (FIG. 6C).

D. Identification of the Developmental Target Protected by the FADD-Caspase-8-FLIP Complex

The phenotypes of the caspase-8, FADD, and FLIP knockouts all show embryonic lethality around e10.5, associated with a defect in yolk sac vascularization. The following studies examine whether early progenitors of vascular endothelium and hematopoietic cells serve as the earliest and most important targets of this developmental defect. In one embodiment, these studies are used to identify additional targets of RIPK3-necrosis and to investigate the signaling pathways engaged in this embryonic lethality.

1. The Developmental Consequences of Tissue-Specific Caspase-8 Ablation

The common lethal phenotype of the caspase-8, FADD, and FLIP single KO mice involves improper vascularization of the yolk sac and lethality around e10.5. The present studies strongly support the idea that the FADD-caspase-8-FLIP complex protects embryonic development from the lethal effects of RIPK3. An early study utilized blastocyst chimeras to map this embryonic defect to the developing heart (3), but subsequent experiments showed that conditional deletion of caspase-8 in cardiomyocytes produced no lethal effects. Instead, the endothelium was identified as the developmental target (30).

This apparent paradox can be resolved by considering that the earliest common precursors of endothelium and blood (hemangioblast/hemogenic endothelium) arise in the mesoderm as it migrates from the primitive streak to extraembryonic and intraembryonic sites. The progenitors “seed” vascular formation and then remodel it. This occurs in several waves creating subsets of endothelium and hematopoietic cells, some of which are transient, forming the embryonic vasculature and primitive blood and others that form the endocardium, major vasculature and the adult blood system. Hematopoietic cells arise in the endothelium of the aorta (around e8-9) and migrate to the yolk sac (33-38) where the progenitors “seed” vascular remodeling from within the vasculature (39). Without these cells the vasculature collapses. Mice lacking TIE2, one of the angiopoietin receptors, show a phenotype very similar to that of the caspase-8-deficient embryos (40, 41). Further, TIE2 marks both endothelium and some hematopoietic cells in adults and in yolk sac (42, 43). It is therefore possible that the cells that depend on TIE2 for differentiation/survival also depend on FADD-caspase8-FLIP, and are the population responsible for vascular remodeling.

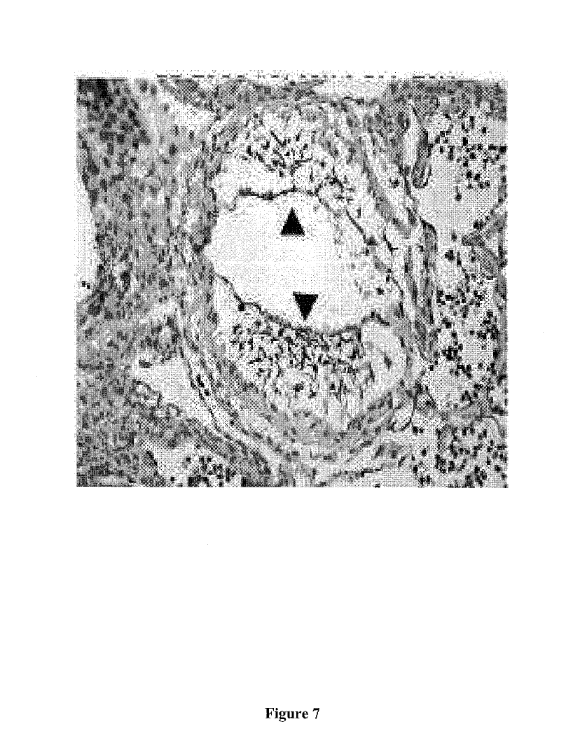

This model was examined in mice expressing CRE on the TIE-2 promoter (42) and a Rosa26p-driven lox-stoplox (LSL)-YFP transgene (43). Embryos (e10-12) obtained from these mice embryos were examined for yellow fluorescent protein (YFP) expression. As expected, expression of YFP was observed in vascular endothelium of the yolk sac (not shown) as well as the “endocardial cushions” of the heart (FIG. 7). The latter may represent the early precursors (34) which are examined in more detail by multicolor immunostaining sections with markers of these cells (CD41, cKit, and Ly6a) (37-39).

To date, crossing these mice to caspase-8^{flox/flox} mice have failed to produce homozygous fl/fl mice carrying TIE2-CRE. This cross may be continued until a result that is significantly different from expected frequency is observed. Staged embryos from these mice are then examined to determine the embryonic defects and condition of the yolk sac and intraembryonic vasculature (as done for FLIP-RIPK3 DKO mice), and the embryos are examined for loss of the YFP⁺ cells, as well as CD41⁺, cKit⁺, or Ly6a⁺ cells (37-39). The cross of TIE2-CRE, LSL-YFP, caspase-8^{fl/+} mice will yield 25% fl/fl embryos and 75% with at least one caspase-8 wild type allele; the latter will serve as controls for YFP expression. All embryos are typed after histology/whole embryo evaluation.) Alternatively, embryos may be disrupted to assess this population (YFP⁺, CD41⁺, cKit⁺, Ly6a⁺) by fluorescence-activated cell sorting (FACS).

Further, these mice are crossed to the RIPK3-null mice to confirm the ability to rescue any embryonic lethality since the caspase-8, RIPK3 DKO is viable. This also provides a valuable control for the analysis of the YFP⁺, CD41⁺, cKit⁺, Ly6a⁺ cells.

If TIE2-CRE does not fully phenocopy the effects seen in the germline deletions, two sets of crosses may be performed to investigate and extend the findings. If the YFP⁺ cell population is lost but vasculature is unaffected, RUNX1-CRE may be used since it has been shown to be expressed by the precursor population (33, 44) and TIE1-CRE, which has been shown to produce embryonic lethality in caspase-8^{flox/flox} mice (30). It should be noted, however, that a careful reading of the latter publication shows that the vascular defect in the TIE1-CRE caspase-8^{flox/flox} mouse is not as dramatic as that of the caspase-8-null. Indeed, TIE2-CRE was chosen for two reasons: a) deletion of TIE2, but not TIE1, (41) most completely phenocopies the caspase-8 and FLIP deletions, and b) TIE1 and TIE2 are co-expressed on some but not all cells (41).

2. Identifying Dying Cells in FLIP-RIPK3-Deficient Animals

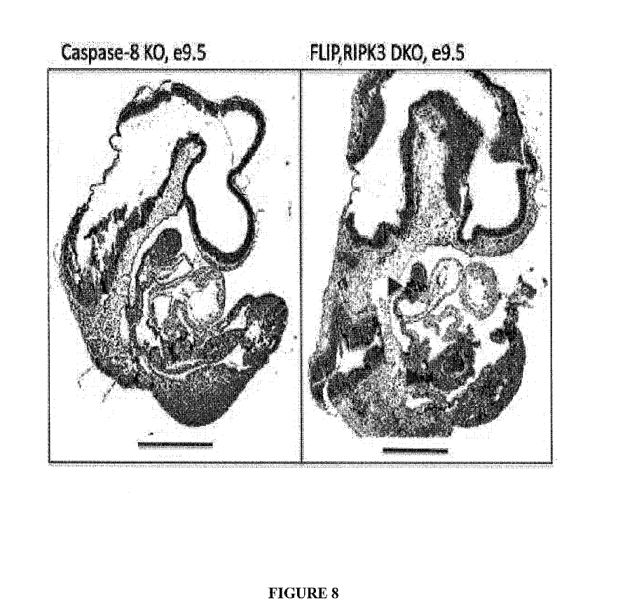

A present model (FIG. 1) strongly suggests that cells that die in caspase-8-null animals do so via RIPK-dependent necrosis. To date, such cell death is difficult to observe unambiguously as there are no specific markers for necrotic death. In contrast, however, the present findings also strongly suggest that the same cells die in FLIP-RIPK3 DKO mice via apoptosis as a consequence of unconstrained caspase-8 homodimer activity (32). This is strongly supported by the normal development of FADD-FLIP-RIPK3 TKO mice. Apoptosis is readily detectable with anti-cleaved caspase-3 (45) or with TUNEL staining. An examination of e9.5 caspase-8-null embryos indicated very little apoptosis (FIG. 8) consistent with published data (31) and therefore limits concerns that apoptosis as a secondary consequence of the developmental defects will obscure our results. However, to further refine this analysis, TIE2-CRE, LSL-YFP mice are crossed to FLIP^{fl/fl}, RIPK3 null mice and apoptosis is examined in the YFP⁺ cells. This comparison allows the identification of dying cells in the defective embryos, and also the extent of secondary apoptosis (as defined above). This determines what tissues are directly (or indirectly) affected by germline deletion of FLIP.

While deletion of FADD, caspase-8, or FLIP is invariably lethal around e10.5; deletion of caspase-8 in specific tissues does not necessarily result in this phenotype. Deletion of caspase-8 in liver (albumin-CRE) (30), heart (myh6-CRE), or neural crest (tyrosine hydoxylase-CRE) causes no developmental defects and deletion in skin (K14-CRE) (46), gut epithelium (villin-CRE) (47), myeloid cells (LysM-CRE) (30), or lymphocytes (CD19-CRE, 1ck-CRE) (2, 17, 48), while producing tissue specific effects, does not result in embryonic lethality. Therefore, widespread apoptosis in FLIP-RIPK3 DKO embryos is not expected. Preliminary studies have found that apoptosis in FLIP-RIPK3 DKO embryos is generally restricted to specific regions, including endothelium and a region of the heart (FIG. 8).

3. Tissue Specific Protection by FADD-Caspase-8-FLIP

Deletion of caspase-8 in several tissues does not necessarily result in severe embryonic defects, and to date, only deletion by TIE1-CRE (30) (and probably TIE2-CRE) causes embryonic lethality at e10.5. While informative this does not formally exclude the possibility that other tissues must be protected from RIPK3-dependent lethality by the FADD-caspase-8-FLIP complex. This is explored by generating animals in which RIPK3 can be conditionally ablated.

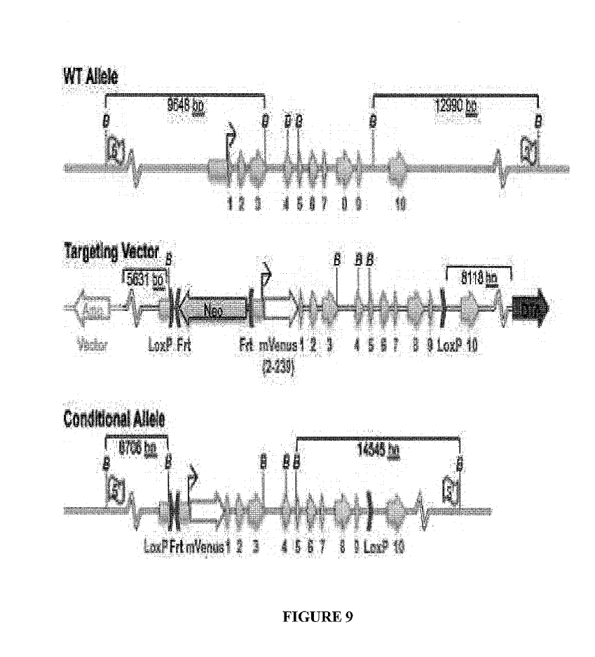

Using the construct outlined in FIG. 9, ES cells are targeted for the generation of engineered mice. These mice offer several useful advantages. First, RIPK3 is expressed as a RIPK3-YFP fusion since this chimeric protein is fully functional in RIPK3-dependent necrosis in vitro (data not shown). In addition, expression of CRE will delete RIPK3. The generation of this animal will be extremely valuable for the studies that follow. A person of skill in the art will recognize that generating this mouse is routine since the RIPK3 germline deletion is developmentally normal (49). Regardless, alternative approaches are discussed below.

The RIPK3-YFP^{fl/fl} mice are then used to generate RIPK3-YFP^{fl/fl}, caspase-8^{fl/-} mice carrying TIE2-CRE (or TIE1-CRE). One subsequent cross will generate both RIPK3-YFP^{fl/fl}, caspase-8^{fl/fl} and RIPK3-YFP^{fl/fl}, caspase-8^{-/-} embryos. Since the germline DKO is viable, conditional deletion of both caspase-8 and RIPK3 in the same tissues is expected to be nonlethal in all cases. The RIPK3-YFP^{fl/fl}, caspase-8^{-/-} embryos will thereby inform whether deletion of RIPK3 in specific tissues (e.g. driven by TIE2-CRE) rescues (or delays) embryonic lethality seen in mice lacking caspase-8. As noted above, based on limited data from conditional deletion of caspase-8 in non-hematopoietic tissues, in one embodiment the specific deletion of RIPK3 in TIE2- (and/or TIE1-) expressing cells significantly delays or prevents embryonic lethality. In addition, these mice indicate whether YFP⁺ cells (expressing RIPK3-YFP) survive in those settings where caspase-8 is deleted in tissues that do not result in embryonic lethality.

Alternative approaches for generating these mice are also contemplated. In one embodiment, lentiviral transgenic mice are generated in which RIPK3-YFP is expressed as a floxable transgene. This is performed with both generalized expression of RIPK3 and, once characterized, with the minimal RIPK3 promoter. The transgene is introduced into zygotes from the homozygous cross of TIE2-CRE, caspase-8^{fl/fl}, RIPK3^{-/-} and deletion of the RIPK3 transgene is expected to be protective in these animals. Crossing these mice to caspase-8, RIPK3 DKO mice is informative, as the effect of expressing RIPK3 in caspase-8-deficient tissues, other than those affected by TIE2-CRE determines the extent to which those tissues contribute to embryonic lethality.

4. FADD-Caspase-8-FLIP Protective Effect Signaling

It is clear that the embryonic lethality caused by FADD or caspase-8 deletion depends on RIPK3 (and that the lethality caused by FLIP deletion likely depends on caspase-8), as demonstrated in the inventors' published (18) and preliminary findings. However, it is not known what developmental signals actually engage both the protective FADD-caspase-8-FLIP complex and the RIPK1-RIPK3-mediated necrotic effect. Several candidate signals are known (or suggested) to engage such signaling. These include TNF-TNFR (10, 11, 18, 46 and 47), TLR-TRIF (26), and perhaps autophagy (16).

Hedrick and colleagues (51), examining caspase-8-deficient T cells, demonstrated that RIPK-dependent necrosis does not involve autophagy since deletion of the essential component ATG7 did not rescue these cells. The inventors have similarly found that germline ablation of ATG7 does not rescue development in caspase-8 deficient mice (data not shown). Similarly, deletion of TRIF does not seem to rescue the caspase-8-null lethality and the inventors' preliminary crosses support this to date (not shown).

Crosses have been initiated to generate TNFR1-null animals lacking caspase-8 or FLIP. To date, no caspase-8 or FLIP homozygous deleted mice have been weaned in this background. However, two examples of late stage embryos (>e17.5) that were TNFR1-FLIP DKO have been identified. This result is significant since such advanced embryonic development in caspase-8, FADD, or FLIP null animals (which invariably die much earlier) have not previously been observed. Embryogenesis of these genotypes in the TNFR1-null background continues to be explored since preliminary results suggest that development is likely to be significantly sustained. This is further supported by recent studies that examined necrosis and inflammation in animals with conditional deletion of FADD (52) or caspase-8 (47) in the intestinal epithelium. In these studies, deletion of TNFR1 greatly ameliorated (but did not completely eliminate) cell death and disease, suggesting that at least in these cells, TNF signaling is an important component of the necrosis. If preliminary results are confirmed it will verify that TNF signaling contributes significantly to the developmental lethality of mice lacking caspase-8 or FLIP. Once elaborated, this will be confirmed in the FADD-null mice. While this is not a full rescue (due, perhaps, to signaling by other TNFR family members) the identification of even one important signaling component of this lethality will greatly increase the power of in vitro systems (e.g., by TNF signaling, (18)) to predict the pathways involved in the embryonic effects.

In one embodiment, the present invention contemplates that the deubiquitinase CYLD is required for engaging TNF-induced RIPK-dependent necrosis in vitro (53), and it has recently been shown that ablation of CYLD activity in intestinal epithelium prevents necrosis due to conditional deletion of FADD in these cells (52). CYLD functions to deubiquitinate RIPK1; an event required for assembly of signaling complexes that include FADD and caspase-8 following TNFR1 ligation (54). The inventors are currently assessing if CYLD is similarly required for RIPK-dependent necrosis induced by CD95 ligation (10) and by ligation of TLR3 or TLR4 (26). If so, it is likely that ablation of CYLD will have a more penetrant effect on survival of the embryos than does TNFR1 deletion.

For example, CYLD^{fl/fl} mice for conditional deletion of CYLD may be used to generate mice with a germline deletion. Although the CYLD KO is reportedly developmentally normal (55, 56), the original deletion may not have ablated the gene. In the event that deletion of the floxed CYLD is not viable, embryos will be examined to determine the developmental stage at which any defects occur. Based on those findings, caspase-8, FADD, and FLIP deficient animals lacking CYLD will be generated to determine if CYLD deletion sustains embryonic development at least to the point that loss of CYLD affects the embryo. In addition TIE2-CRE, caspase-8^{fl/fl}, CYLD^{fl/fl} mice will be generated, which may be necessary if there is earlier lethality in the full KO.

CYLD is a tumor suppressor in some skin cancers and is thought to regulate NF-kB activation (57). The in vitro finding herein, and the potential for in vivo assessments, may indicate that it also functions to promote RIPK-dependent necrosis when FADD-caspase-8-FLIP activity is limited. In one embodiment, the present invention contemplates that regulation of CYLD expression and/or function impacts oncogenesis through control of RIPK-dependent necrosis. These studies provide an informed understanding of how, and in what tissues, disruption of the regulation of RIPK1-RIPK3 interactions by FADD-caspase-8-FLIP influences development. Should additional important tissues be identified, future studies will explore specific ablation of the key genes in these tissues using available transgenic CRE or, if necessary, generating new transgenic animals.

5. Regulation of RIPK-Dependent Necrosis in Oncogenesis.

While evasion of apoptosis is a well-established hallmark of cancer, the role of RIPK-dependent necrosis in tumor suppression has not been explored. A six-nucleotide deletion polymorphism in the caspase-8 promoter has been identified that destroys an SP1 binding site resulting in decreased caspase-8 expression. In a large case-controlled study this was associated with a dramatic reduction in incidence of a wide variety of cancers, including lung, esophageal, colorectal, breast, and cervical (59). The study also cites unpublished independent results showing reduced risk of cutaneous melanoma and squamous cell carcinoma while a subsequent study found a similar association with head and neck cancers (60). This suggests that reduction in caspase-8 can inhibit oncogenesis in many tissues. In striking contrast, caspase-8, present on human chromosomes 2q33-34, is frequently deleted, mutated, or silenced in neuroblastoma (61), small cell lung carcinoma (62), medulloblastoma (63), some colon carcinomas (64), and in primary glioblastoma cell lines (65). This creates a paradox in which oncogenesis engages RIPK-dependent necrosis that is inhibited by caspase-8, but loss of both caspase-8 (and its associated apoptosis) and the RIPK-dependent necrosis pathway may promote oncogenesis. A survey using Oncomine suggests that decreased RIPK3 expression is associated with poor prognosis in several leukemias (particularly chronic lymphocytic leukemia; CLL) and therefore loss of RIPK3 expression may indeed permit caspase-8 loss with additional effects on apoptosis resistance.

6. Role of the Apoptotic and Anti-Necrotic Functions of the FADD-Caspase-8-FLIP Complex in Oncogenesis or its Suppression

Wallach and colleagues (66) examined the transformation of MEF with SV40, and found that in the absence of caspase-8, cells acquired anchorage independence (growth in soft agar) and tumorgenicity in vivo at a higher rate than wild type cells. In our experience early passage MEF from caspase-8-null mice are sensitive to TNF-induced RIPK3-dependent necrosis (18), however after immortalization with SV40 or E1A/Ras those cells often lose RIPK3 expression along with this sensitivity (data not shown). It is therefore possible that such loss of RIPK3 expression accompanies the transformation (66).

Early passage MEF from caspase-8, FADD, and FLIP single KO embryos are generated and immortalize with SV40 versus E1A/Ras. Colony formation is assessed in soft agar over time and surviving clones are examined for expression of TNFR1, RIPK1, RIPK3, and CYLD and sensitivity to TNF-induced necrosis versus apoptosis (18). Loss of expression of any one of these molecules is likely to prevent RIPK-dependent necrosis, at least in response to TNF. Indeed, a survey of several cell lines has shown that RIPK3 expression is often absent even when the tissues from which the lines are derived express RIPK3.

The role of these important components in the derived cell lines are determined by enforcing expression of those that are lost to determine their effects on TNF-induced, RIPK-dependent necrosis. Retroviral transduction with an IRES YFP reporter is employed to ensure expression. Further, cells that are transduced for loss of YFP are examined for expression in soft agar and in flank tumors. Preliminary studies (not shown) demonstrate that cells with chemically-enforced expression of RIPK3 often lose it over passage. This is explored for RIPK1 and CYLD and it is determined whether this loss of expression is exacerbated in cells lacking caspase-8 or FADD.

It is possible that loss of RIPK-dependent necrosis may occur without loss of those molecules being examined. It is known, for example, that RIPK1 is regulated via complex ubiquitination events (67, 68) and changes in these could have an impact on sensitivity to necrosis (69). Cells for which loss of sensitivity to necrosis is not readily explained are examined to determine if they are capable of undergoing necrosis in response to oligomerized RIPK3 using the inducible RIPK3 system described herein. This determines if resistance is upstream or downstream of RIPK3 activation.

A second approach compares the transformation of MEF from wild type, RIPK3 KO, caspase-8 KO, FADD KO, RIPK3-caspase-8 DKO and RIPK3-FADD DKO embryos. As CYLD KO (and caspase-8-CYLD DKO) embryos are generated, MEF from these animals are employed as well. This identifies the extent to which disruption of RIPK-dependent necrosis facilitates transformation and how loss of FADD or caspase-8 in the absence of such necrosis contributes to the process.

7. Effect of CYLD Loss on Oncogenesis and the Role of RIPK-Dependent Necrosis

CYLD is classified as a tumor suppressor and germline mutations in CYLD are strongly associated with occurrence of human hair follicle tumors in familial cylindromatosis and multiple familial trichoepithelioma (57). Mutations or deletions of CYLD have been observed in multiple myeloma (70) and Hodgkin's Lymphoma (71) as well as in kidney cancer, hepatocellular carcinoma and uterine cervix carcinoma (72-74). In mice, deletion of CYLD does not lead to spontaneous cancer but such mice are susceptible to chemically-induced skin and colon cancers (56, 75). Other cancer models have not been examined to date. It is intriguing, however, that mice lacking CYLD show B cell hyperplasia (76). Aside from its role in promoting RIPK-dependent necrosis CYLD has a variety of other activities, including inhibition of NF-kB activation through its removal of K63-Ub on either RIPK1 (54) or BCL-3 (77). CYLD is also thought to regulate the cell cycle by promoting progression through M phase (78).

The role of CYLD in RIPK-dependent necrosis has, to date, only been examined in response to TNF in vitro (53) and in an in vivo system in which TNF plays a major role (52). It is possible, however, that CYLD plays a more central role in promoting RIPK-dependent necrosis through its deubiquination of K63-Ub-RIPK1 (54). RIPK-dependent necrosis is examined by engagement of TLR3 or TLR4 in macrophages (26). This occurs via TRIF-RIPK1 interaction and is independent of TNF (26). Bone marrow derived macrophages can be routinely generated from wild type and KO mice that are readily susceptible to siRNA-mediated knockdown (79). siRNA is employed to knock down CYLD expression in macrophages and assess necrosis induced by LPS or polyI:C plus zVAD-fmk versus TNF/zVADfmk. If necrosis is dependent on CYLD in both settings, the effect of CYLD knockdown will be examined in TNFR1-null macrophages. The RIPK1 inhibitor nec1 is used as a control.

These results are further extended using the two inducible systems described herein. In these systems, rapid necrosis is induced by addition of dimerizer to cells expressing RIPK1-FRB and RIPK3-FKBP1x (RIPK1 dependent) or expressing RIPK3^{ΔC}-FKBP2x (RIPK1 independent). Wild type with or without CYLD knockdown, as well as CYLD-null MEF (I enforced CYLD) expressing these constructs are subjected to dimerizer and necrosis is assessed. This will determine if deubiquitination of RIPK1 is a general requirement for RIPK1-dependent necrosis.

Together these experiments are informative of the general role of CYLD in RIPK-dependent necrosis. It is possible, for example, that CYLD is only required in settings where RIPK1 associates with receptor complexes (perhaps only TNFR1) and is not a general requirement for RIPK1 function.

As discussed above, transformed caspase-8 and FADD deficient mice are examined in MEF for expression of CYLD (as well as RIPK1 and RIPK3). These studies are extended to determine if MEF lacking CYLD (from our knockout mice) are more readily transformed in vitro. Expression of CYLD (with retroviral IRES YFP, as above) is enforced to determine if this inhibits the transformed phenotype. To determine if any effect of CYLD deletion is via loss of RIPK-dependent necrosis it will be determined whether any loss of expressed CYLD is ameliorated by inhibition of RIPK1 (with necrostatin) or knockdown of RIPK3 (18).

8. FADD-Caspase-8-FLIP Mediated Protection in a Model of B Cell Lymphoma

While investigations of oncogenic transformation in MEF provide some insights into the transformation process it does not model cancer. Therefore, Philadelphia chromosome-positive (Ph⁺) B-cell acute lymphoblastic leukemia (Ph⁺ B-ALL) is employed to investigate the role of protection from RIPK-dependent necrosis in a cancer model. This model employs retroviral transduction of BCR-ABL (p185 or p210) into pre-B cells (80). Wild type pre-B cells transduced with p185 produce lymphoma in immunodeficient mice within 30 days of transfer (p210 is somewhat slower requiring approx 60 days), and dramatic acceleration is observed in some genetic settings (80). Therefore, this model is very useful for in vitro and in vivo studies without requiring extensive additional crosses.

Similar to the MEF studies, the effects of caspase-8 RIPK3 deletion on transformation are investigated in this system. As expected, B cell development is observed in CD19-CRE caspase-8^{fl/fl} mice in the absence of caspase-8 (41). These mice are currently being crossed with the RIPK3 KO. Bone marrow pre-B cells are transduced with BCR-ABL (p180 or p210) and proliferation in vitro is monitored as described (80). Cells are transferred to Rag-null recipients to assess lymphomagenesis. Transformed caspase-8-deficient cells are examined for expression of TNFR1, RIPK1, RIPK3, and CYLD, as outlined above.

Should the status of caspase-8±RIPK3 have an impact on lymphomagenesis in this model, these findings will be extended to FADD and CYLD, as discussed above. As lack of CYLD leads to B cell hyperactivity and hyperplasia (76, 77), these studies will be highly informative.

E. Caspase-8 Loss in Human Neuroblastoma Lines

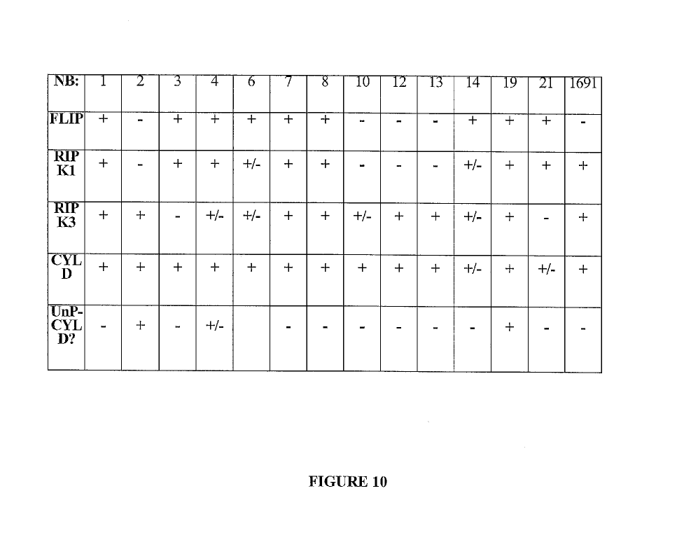

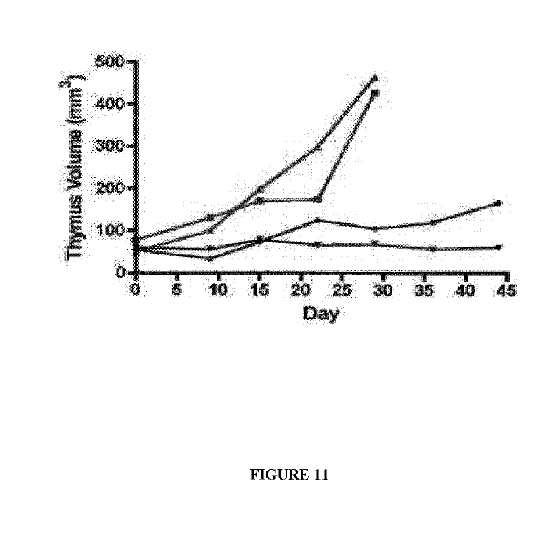

Human neuroblastomas (NB) often delete or silence expression of caspase-8 but NB lines do not show susceptibility to cell death induced by ligation of TNFR or CD95 (61). This presents a paradox when considering the role of caspase-8 (in the FADD-caspase-8-FLIP complex) in protection against RIPK-necrosis. How do these cells avoid such necrotic death? Several NB lines are profiled for expression of RIPK1 and RIPK3 (FIG. 10). Preliminary experiments demonstrate that several cell lines deficient in caspase-8 expression show considerable down regulation of RIPK1, RIPK3, or both. Strikingly, however, some caspase-8 deficient lines (e.g., NB1, NB7, NB8, NB15, NB19) show strong RIPK1 and RIPK3 expression despite being resistant to death receptor ligation (61, and results not shown). These results are repeated and extended to examine protein and mRNA levels of caspase-8, RIPK1, RIPK3, and CYLD as well as sensitivity to TNF, CD95-ligand, and TRAIL±zVAD-fmk (see below).

CYLD is regulated by phosphorylation by IKKε resulting in decreased CYLD deubiquitinase activity (81). In most cell lines only the higher (possibly phosphorylated (81)) form of CYLD is observed. This analysis is extended to assess CYLD phosphorylation, using a commercially available phospho-S418 CYLD antibody (Cell Signal).

As with the MEF studies outlined above, the present data show that chemically-enforced expression of any of the above components of RIPK-necrosis (RIPK1, RIPK3, CYLD) were found to be deficient in the cells to assess if this restores sensitivity to necrosis induced by TNF (as well as CD95-L and TRAIL). In the event that CYLD is found to be phosphorylated in some of these lines the effects of expression of CYLD S418A (preventing such inhibition) in these cells is examined.

Unlike the murine cell lines, human cells have the potential to express caspase-10 (rodents do not have a caspase-10 gene). While caspase-10 is closely related to caspase-8 there is currently no information as to whether or not it can participate in protection from RIPK-dependent necrosis. Cell lines may be found that express RIPK1 and RIPK3 but not caspase-8 that are sensitized by zVAD-fmk to TNF-induced necrosis, where caspase-10 will be knocked down to determine if it is responsible for protecting these cells from RIPK-necrosis. If so, caspase-10 will be expressed and mutated to prevent siRNA knockdown to confirm that it is in fact be responsible.

It should be noted that a mutation family in caspase-8 associated with Acute Lymphoproliferative Syndrome (82) suggests that caspase-8 is not required for human development (30). However, in vitro studies show that caspase-8 appears to be required for proper differentiation of human villous trophoblast (7, 8); a point that should be readdressed in light of the findings on RIPK-dependent necrosis. The human mutation R248W present between β1 and α1 near the substrate (83) is being examined to determine how this affects activity of the caspase-8 homodimer (pro-apoptotic) and the caspase-8-FLIP heterodimer (anti-necrotic). It remains possible that this mutation does not prevent the protective effects. Results will be informative of how important the mutant is for interpreting of the role of caspase-8 (or caspase-10) in human development and cancer.

It should be noted that virtually nothing is known about the role of RIPK-dependent necrosis or its inhibition by FADD-caspase-8-FLIP in any model of cellular transformation. These studies represent the first in-roads towards gaining an understanding in this regard.

F. RIPK-Dependent Necrosis Therapies

Many approaches to cancer therapy seek to promote apoptosis, which may or may not promote ancillary antitumor immunity. In one embodiment, shifting signals to RIPK-necrosis prevents iatrogenic damage in tissues resistant to this form of death (e.g., liver) while promoting an inflammatory mode of tumor cell death. “Pure” RIPK3-induced necrosis versus apoptosis is modeled to examine the anti-tumor consequences and to explore a counter-intuitive approach to triggering RIPK-dependent necrosis in autochthonous and grafted tumors by death receptor ligation in vivo. In another embodiment, studies are directed to determining whether tumor neovasculature is targeted by RIPK-dependent necrosis.

Results demonstrating that the lethal effects of deleting caspase-8, FADD, or FLIP can be explained and rescued by deletion of RIPK3 (in the case of caspase-8 and FADD) or both FADD and RIPK3 (in the case of FLIP) have fundamentally changed previous notions regarding the functions of these molecules in the control of cell death. As such, the studies proposed herein extend to the field of development and cancer.

Some, but not all, tumor cell lines are deficient in RIPK3 expression and therefore resistant to RIPK-dependent necrosis (14). However, whole genome sequencing of 150 pediatric cancers in the Washington University Pediatric Cancer Genome Project has not revealed any examples of RIPK1 or RIPK3 deletion or mutation. This raises the possibility that RIPK-necrosis can be engaged in many tumors provided RIPK1-RIPK3 expression persists or can be induced. Further, because RIPK-dependent necrosis appears to be inflammatory (85) it is possible that induction of RIPK-necrosis in even a subset of tumor cells may promote effective anti-cancer immune responses. In one embodiment, the present invention contemplates exploiting this novel form of cell death as a cancer treatment.

G. RIPK-Dependent Necrosis Advantages Over Apoptosis

While earlier studies suggested that necrosis and apoptosis can be discriminated as “immunogenic” and “non-immunogenic” (or tolerogenic) cell death, respectively, more recent evaluations have shown that in some instances apoptotic cell death can promote anti-tumor immune responses (86). Nevertheless, little is known regarding the inflammatory effects of RIPK-dependent necrosis and whether it can facilitate tumor destruction.

Some of the systems used herein were developed for the induction of “pure” RIPK-dependent necrosis and caspase-8-induced apoptosis in cells (27). Both systems rely on expression of chimeric proteins containing FKBP domains that permit dimerization (or, in the case of multiple FKBP sites, oligomerization) upon addition of a bivalent binding drug (Clontech (87)). These constructs are shown in FIG. 6A. Stable lines of SV40-transformed MEF have also been created expressing each of these constructs. These cells are injected into syngeneic immunodeficient (Rag-2-null) and immunocompetent mice to form flank tumors prior to administration of the dimerizer as described previously (88). The injected dimerizer is titrated to determine the effective dose (in one embodiment 50 μg/mouse or less) (88) and the resulting tumors monitored by ultrasound. Incomplete cell death is modeled by mixing the cells with control transformed MEF (each marked with fluorescent proteins plus luciferase) at frequencies ranging from 1:1 to 1:10. Once tumors are established the animals are intraperitoneally (88) or intravenously (89) infused with the dimerizing agent. If either form of cell death (apoptosis or RIPK-dependent necrosis) engages innate and/or adaptive anti-tumor responses, an effect on tumors that include cells without the constructs is expected. Tumors are monitored using both ultrasound and the luciferase reaction, with the percentage of controls versus engineered cells assessed by flow cytometry using the fluorescent markers. In addition, circulating inflammatory cytokines are monitored (Luminex) and histology is performed on the tumors to assess inflammation.

These studies are extended using B cell lymphoma lines derived from Eμ-myc lymphomas as previously described (90). These cells are transduced with the above constructs and implanted in immunodeficient and syngeneic immunocompetent mice, followed by similar experiments to those described above.

H. RIPK-Dependent Tumor Necrosis

In the original descriptions of cell death induced by ligation (using agonistic antibody) of CD95 a remarkably potent anti-tumor effect was noted in xenograft models (91). Any hopes of using CD95 ligation as an anti-tumor therapy were quickly dashed by the realization that administration of agonistic anti-CD95 antibody (92) is extremely toxic due to fulminant liver destruction. Although it was found that pharmacologic inhibition of caspases protects animals from the toxic effects of CD95 ligation (93), it was assumed that this would similarly prevent any anti-tumor effects and therapeutic use of CD95 agonists for therapy was dismissed.

With the realization that ligation of TNFR-family receptors, including CD95, can engage RIPK-dependent necrosis, the possibility that combined treatment with agonistic anti-CD95 and caspase inhibitors may have some beneficial effects re-emerges. This previously counter-intuitive idea is examined using a mouse oncogene model. Mice with an engineered p53 mutation lacking the proline-rich domain of p53 (94), and harboring an Eμ-myc transgene, develop B cell lymphoma at 100% frequency within 3 months of birth (data not shown). In a pilot experiment, mice with detectable tumors at 5 weeks (determined by ultrasound, and characteristic of this lymphoma) were given a short course (5 days) of agonistic anti-CD95 (Jo2) and zVAD-fmk (FIG. 11). As expected zVAD-fmk completely protected the animals from the lethal effects of anti-CD95 in vivo. Remarkably, however, tumors in these animals did not progress while control mice died. This experiment was performed twice with identical results. That study is repeated and extended using Eμ-myc transgenic mice with the p53^{ΔPP} mutation, as well as mice with the more rapidly oncogenic p53^{+/-} genotype; although tumors in the latter group may be too advanced at the time of weaning for beneficial effects to be seen. Controls using zVAD-fmk alone are also included.

While there are caspase inhibitors in clinical trial (95, 96), studies suggest that these have short half-lives in circulation and localize to the liver (97, 98). Indeed, ablation of caspase-8 specifically in the liver is sufficient to abrogate the lethal effects of CD95 ligation (30). It is therefore possible that any effects of anti-CD95/zVADfmk on tumor growth will not be due to induction of RIPK-dependent necrosis, but rather apoptosis in the tumor. To address this Eμ-myc lymphoma lines are injected into animals with liver specific deletion of caspase-8 (albumin-CRE caspase-8^{fl/fl}). These mice are resistant to the lethal effects of CD95 ligation in vivo. These animals are used to assess the effects of antiCD95 with or without zVAD-fmk (as well as zVAD-fmk alone) to determine if caspase inhibition is required. This will indicate whether apoptosis or RIPK-dependent necrosis (i.e., with zVAD-fmk) is the required mode of killing in the tumor cells. As an alternative approach, the effects of shRNA knockdown of RIPK3 are investigated in these lines upon treatment with ant-CD95/zVAD-fmk.

The Ph⁺ B-ALL model is further exploited to determine if these cells are susceptible to necrotic death induced by anti-CD95/zVAD-fmk in vitro Animals transferred with caspase-8-null, RIPK3-null, or DKO cells transduced with BCR-ABL are treated with anti-CD95/zVAD-fmk. If RIPK-dependent necrosis is required for the effects of this treatment then any should be dependent on the presence of RIPK3 in these leukemias.

It is also possible in at least some cases that the effects of anti-CD95/zVAD-fmk treatment are not on the tumor itself but rather on the neo-vasculature recruited by the tumor. This would be consistent with the reported findings in the developing embryo that are supported by studies suggesting that anti-CD95 mediates its effects via endothelial destruction (99, 100). If this is the case then the effects will depend on the genotype of the host rather than the genotype of the tumor. This is examined with both transformed MEF (as flank tumors) and the Eμ-myc lymphoma lines (above) introduced into wild type, RIPK3 KO, or caspase-8, RIPK3 DKO mice.

### VI. RIPK3 Dimer-Induced Necroptosis

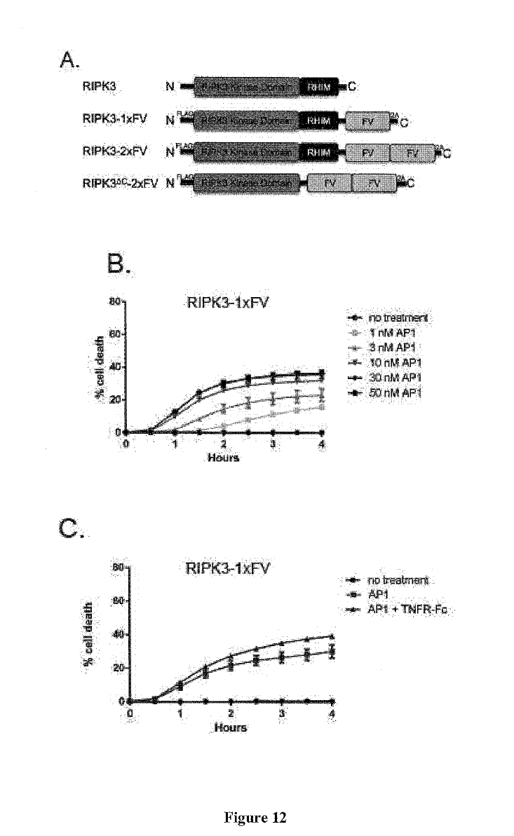

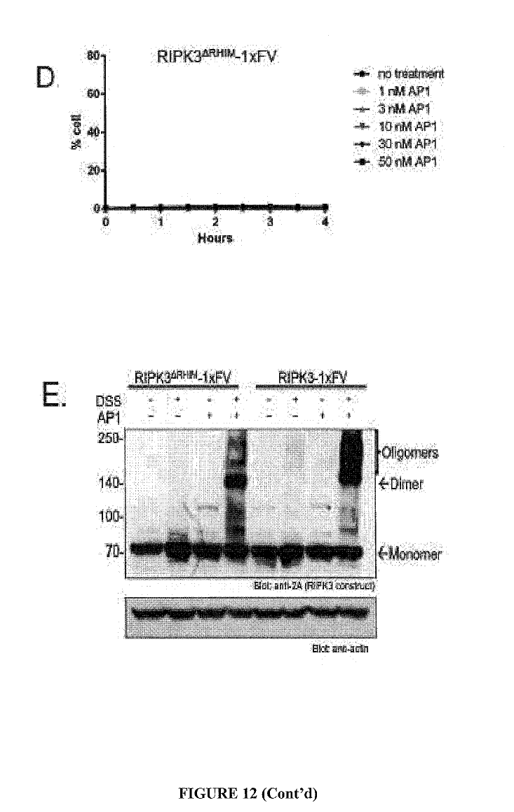

In an effort to gain insight into the mechanism by which RIPK3 is activated, a chimeric protein was created composed of murine RIPK3 fused to a single copy of a mutated FKBP (F36V) (hereafter “Fv domain”), a protein domain that binds with high affinity to a synthetic bivalent homologue of rapamycin, here called “AP1”. FIG. 12A.

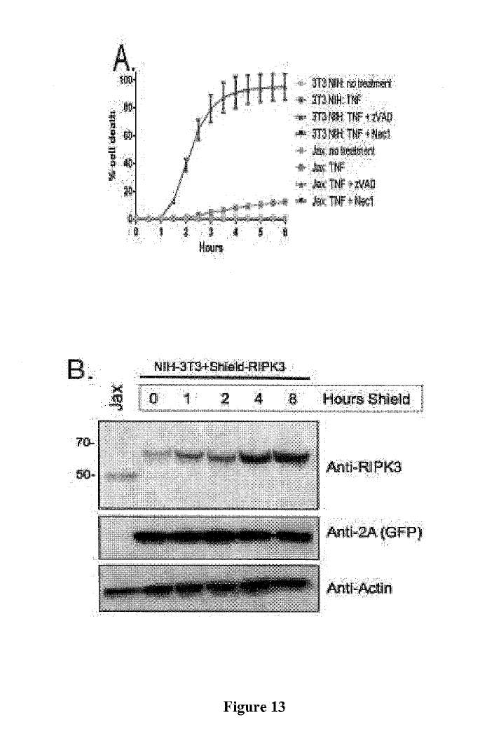

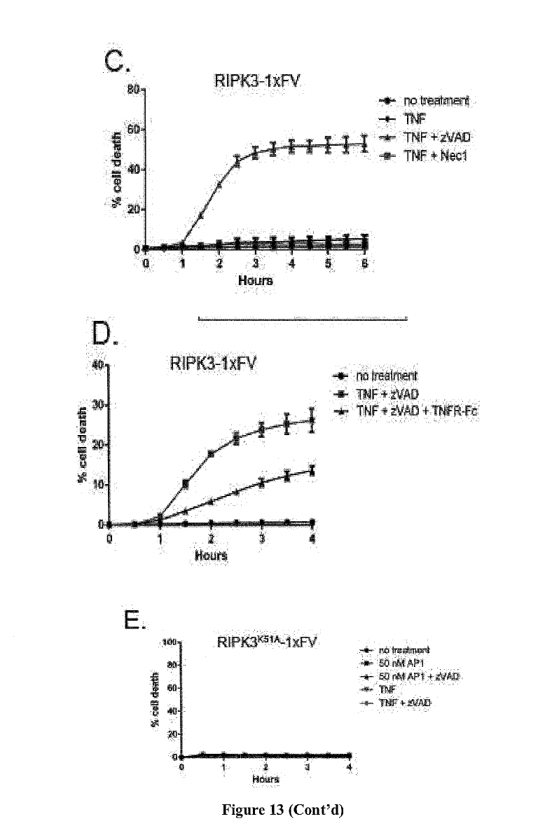

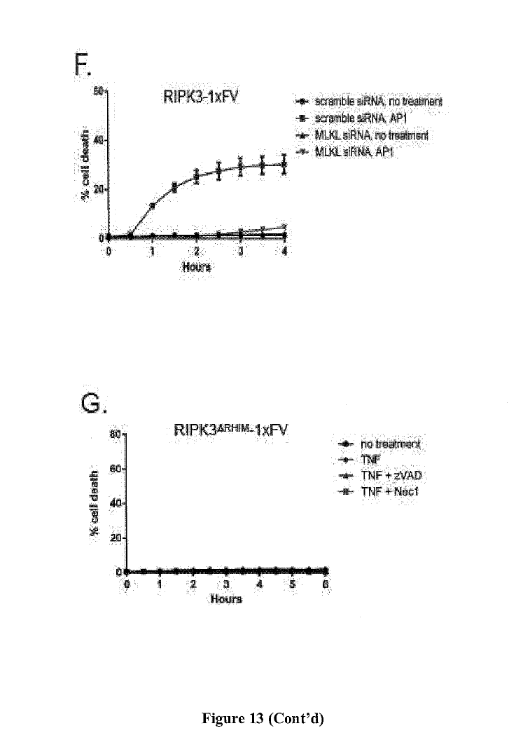

Fv domains rapidly dimerize in response to AP1 treatment that facilitated an investigation of the protein-protein interactions involved in RIPK3 activation and necroptosis (27a). For example, an Fv domain was appended to a C-terminus of RIPK3, that is adjacent to the RHIM domain, in an effort to mimic in situ RHIM-dependent interactions that define RIPK3 activation. RIPK3-1xFv was expressed in NIH-3T3 cells, a cell line that lacks endogenous RIPK3 expression and is therefore unresponsive to TNF-induced necroptosis. See, FIGS. 13A and 13B. Cell death responses of NIH-3T3 cells were quantified by construct expression over time using the IncuCyte imaging system, which allows precise quantification of cell death with high temporal resolution. For example, representative images were acquired from an IncuCyte Imager (data not shown). These images were NIH-3T3 cells stably expressing RIPK3-1xFV, treated with 10 nM AP1 (see FIG. 1B for a quantified graphical representation of this cell death). One set of images show a field of these cells undergoing RIPK3-dependent cell death; the media contains the cell impermeable DNA binding dye Sytox Green, which marks cells that have lost membrane integrity in green. Another set of images depicts the same image set, following analysis by the IncuCyte software package. This software counts each green (dead) cell in each image; counted cells are surrounded by purple halos. Each trace shown in this manuscript depicts data averaged from at least 3 independently treated wells of cells, each of which is imaged in at least 4 separate fields. Each experiment shown is representative of at least 3 separate replicates. All experiments on stably transduced cells is representative of at least three separate replicates performed on each of at least two distinct, independently-derived stable lines.

Expression of RIPK3-1xFv sensitized these cells to necroptosis induced by the combination of TNF and a caspase inhibitor (zVAD) in a manner analogous to that observed in Jax cells, a murine fibroblast line that expresses endogenous RIPK3. See, FIG. 13C. These data indicated that the RIPK3-1xFv construct could be faithfully activated by the well-described pathway of TNF receptor-driven cell death.

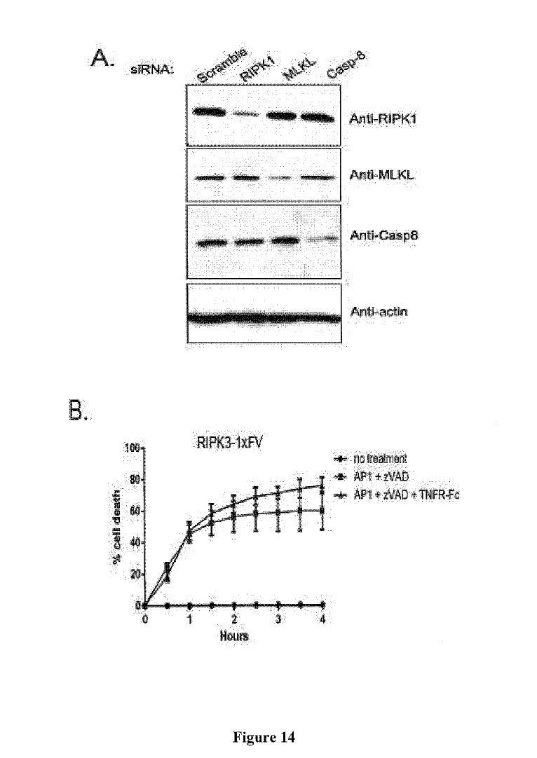

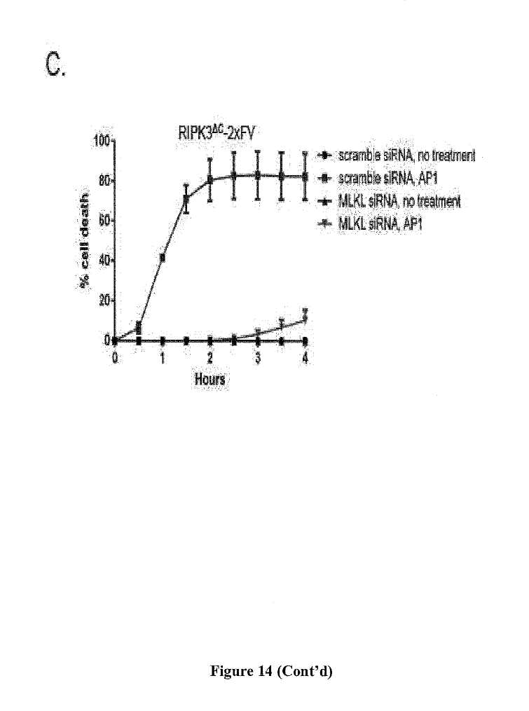

AP1 was then added to these cells in the absence of TNF, to test the effect of chemically-enforced RIPK3 dimerization in the cytosol. Upon AP1 addition, the cells underwent a limited RIPK3 activation and necroptosis in a manner that depended upon the concentration of AP1 added. See FIG. 12B. To ensure that the cell death observed upon dimerizer addition was not influenced by autocrine TNF production, they were treated with the TNF blocking reagent TNFR-Fc. While TNFR-Fc efficiently inhibited TNF-induced necroptosis in these cells, it did not affect AP1-induced cell death. See, FIG. 13D and FIG. 12C. However, dimer-induced cell death did require the kinase activity of RIPK3, as well as a downstream mediator of necroptosis (e.g., MLKL). See, FIG. 12E, FIG. 13F and FIG. 14A. Together, these data confirm that RIPK3 dimerization leads to necroptosis via direct activation of RIPK3. The RIP kinases interact via RHIM domains, and mutation of this domain in RIPK3 renders it unresponsive to receptor-driven necroptosis. See, FIG. 13G, 17a.

The RHIM domain was tested in regards to RIPK3 activation via a dimerization assay. Surprisingly, despite the fact that RIPK3 interaction was being induced via AP1-mediated homodimerization, a mutated version of RIPK3-1xFv, in which an RHIM amino acid sequence VQIG was modified to AAAA (RIPK3^{ΔRHIM}-1xFv) failed to trigger cell death following AP1 treatment. See, FIG. 12D. Recent structural evidence demonstrated that a RHIM domain of RIPK3 forms amyloid-like oligomers during RIPK3 activation. It was therefore hypothesized that while RIPK3 dimerization itself is insufficient for its activation, it may “seed” RHIM oligomers that recruit both RIPK1 and additional molecules of RIPK3, and whose propagation allows RIPK3 activation.

To directly test this hypothesis, and capture evidence of RIPK3 dimers or oligomers, a DSS crosslinking experiment was performed on cells expressing RIPK3^{ΔRHIM}-1xFv or RIPK3-1xFv following dimerizer treatment. Consistent with the hypothesis, RIPK3^{ΔRHIM}-1xFv was predominantly found in a gel-shifted complex in a form consistent with dimer formation, whereas RIPK3-1xFv was present in a combination of dimers and larger oligomeric complexes. See, FIG. 12E.

### VII. RIPK1 and Caspase 8 Control of RHIM-Dependent RIPK3 Complex Formation

Current models of receptor-driven necroptosis involve the scaffolding and activation of RIPK1 at a plasma membrane receptor, followed by its translocation to the cytosol and the recruitment of both caspase-8 and RIPK3 into a “necrosome” complex (1a). It has been reported that RIPK1 can activate RIPK3 in this complex, while caspase-8 can suppress this activation (5a, 25a, and 26a).

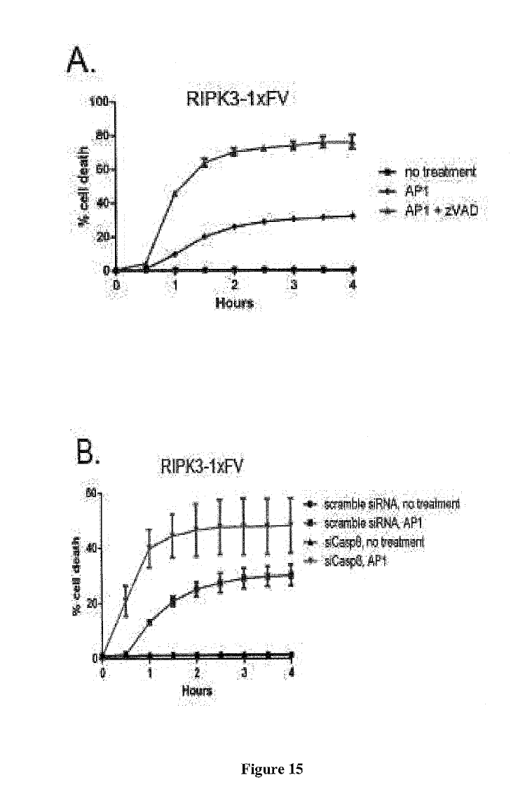

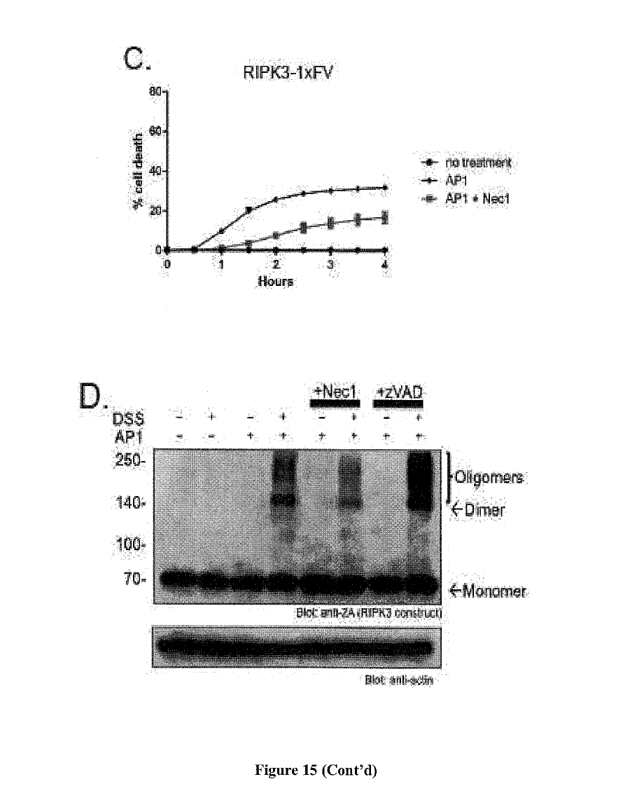

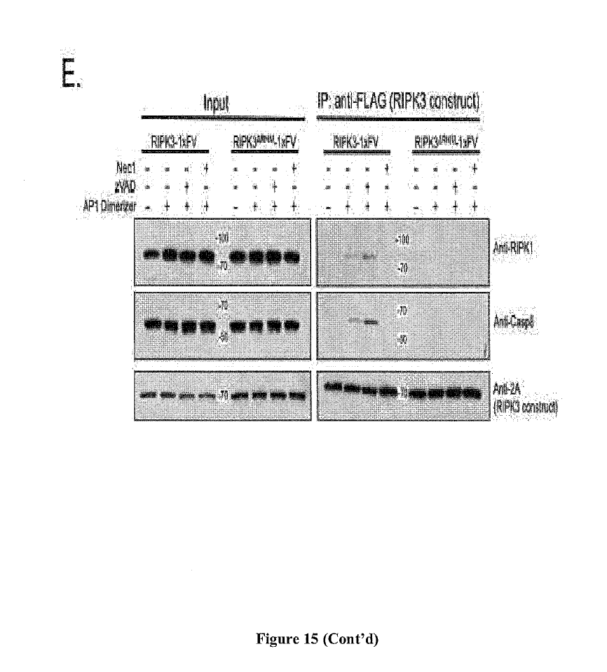

The data herein investigated whether similar dynamics might influence the receptor-independent formation and propagation of RIPK3 complexes triggered by RIPK3 dimerization. Surprisingly, it was found that addition of a caspase inhibitor (zVAD), or siRNA-mediated caspase-8 knockdown notably increased the rate and magnitude of necroptosis triggered by RIPK3-1xFv dimerization. See, FIGS. 15A, 14A and 14B, respectively. These data are analogous to that observed with TNF-driven RIPK3 activation in these cells. See, FIG. 13B.

However, these effects were independent of any TNF receptor engagement, as increased cell death observed upon treatment with AP1 and zVAD were unaffected by the TNF blocking reagent TNFR1-Fc. See, FIG. 14B. Conversely, treatment of cells expressing RIPK3-1xFv with the RIPK1 inhibitor necrostatin-1 (Nec1) (3a), which blocks TNF-induced necroptosis in these cells, suppressed RIPK3 dimerization-induced cell death. See, FIG. 13C and FIG. 15C, respectively. These findings indicate that caspase-8 and RIPK1 may intrinsically regulate the initiation and propagation of RIPK3 oligomers in the cytosol, independent of TNF receptor-mediated signaling pathways.

To more directly test this idea, disuccinimidyl suberate (DSS) crosslinking experiments were performed to visualize the formation and stability of RIPK3 complexes following RIPK3 dimerization, when either caspase-8 or RIPK1 were inhibited. See, FIG. 15D. The data showed that dimerization of RIPK3, in the presence of zVAD, led to an increased shift of RIPK3 into higher molecular weight complexes consistent with RIPK3 oligomers, while RIPK3 dimerization in the presence of Nec1 diminished the appearance of these complexes. As further confirmation, RIPK3-1xFv or RIPK3^{ΔRHIM}-1xFv was immunoprecipitated from cells treated with AP1 in the presence of Nec1 or zVAD.

Previous reports have shown that caspase inhibition can stabilize a caspase-8 and RIPK1 containing necrosome complex following TNF treatment. (25a, 32a) The data presented herein also shows a similar phenomenon following RIPK3 dimerization. See, FIG. 15E. Dimerization itself led to limited recruitment or RIPK1 and caspase-8 to the RIPK3 complex, while addition of zVAD increased association of these proteins. Inhibition of RIPK1 by Nec1, by contrast, eliminated formation of stable RIPK1- and caspase-8 containing complexes. Similarly, mutation of the RHIM domain of RIPK3 prevented necrosome formation upon RIPK3 dimerization (e.g., for example, RIPK3^{ΔRHIM}). Together, these data show that RIPK3 dimerization, in the absence of receptor signaling, is sufficient to nucleate formation of a RHIM-dependent necrosome. Further, necroptotic signaling from this complex is potentiated by the kinase activity of RIPK1, and inhibited by caspase-8 in a manner analogous to that observed during TNF-mediated necroptosis.

### VIII. RIPK3 Oligomerization Triggers Necroptosis

The above data supports the hypothesis that RIPK3 oligomerization plays a role in RIPK3 activation, and that RIPK1 and caspase-8 act by regulating the initiation and propagation of RIPK3 oligomers. Accordingly, chemically-induced oligomerization (rather than dimerization) of RIPK3 should eliminate the ability of RIPK1 and caspase-8 to control this process.

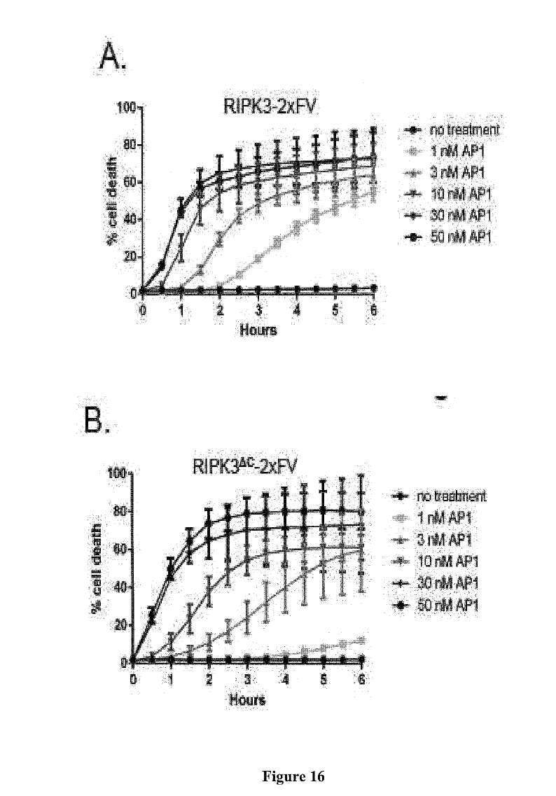

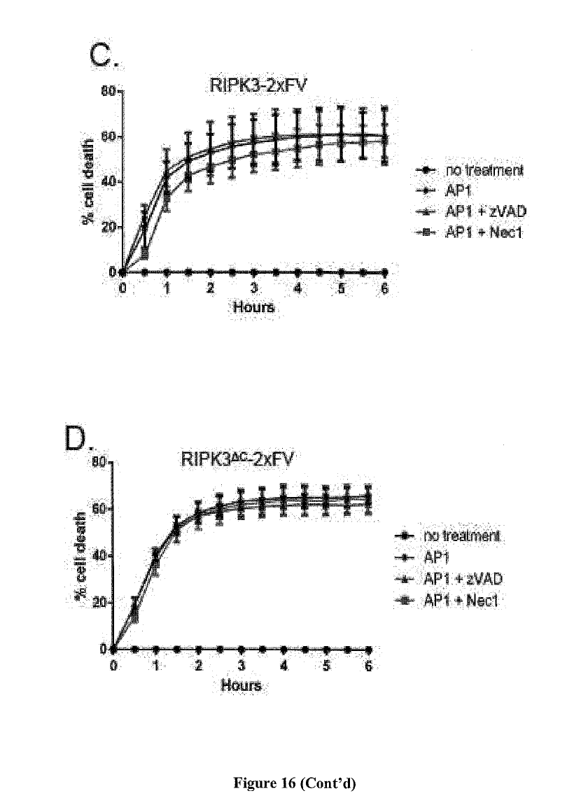

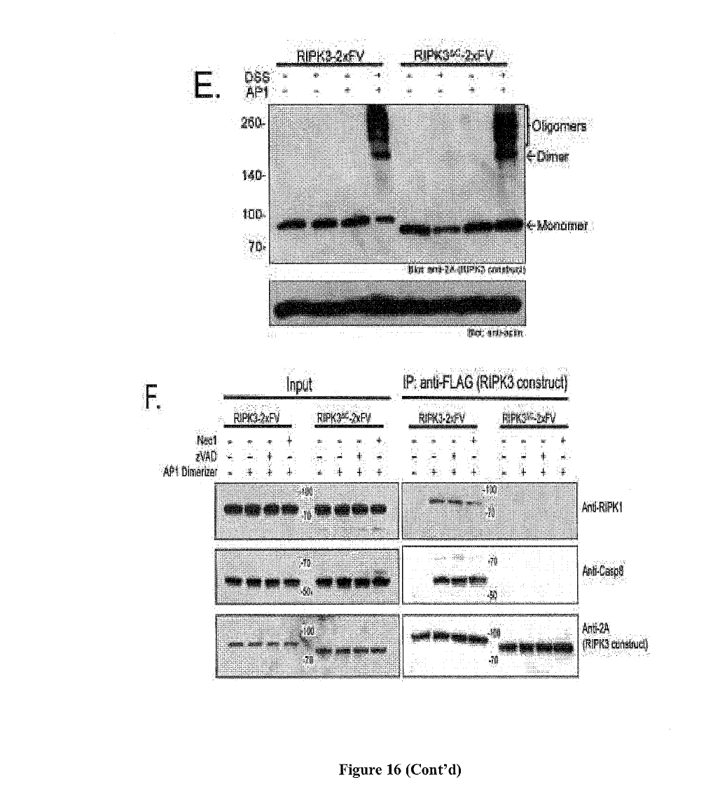

Consequently, two Fv domains were attached to the C-terminus of RIPK3, creating a RIPK3-2xFv construct, with the goal of promoting AP1-induced crosslinking and oligomerization independent of the RHIM domain. See, FIG. 12A. Notably, a faster and more robust necroptotic response was observed in cells expressing RIPK3-2xFv, to a degree comparable to that observed upon dimerization of RIPK3-1xFv following caspase-8 inhibition or knockdown. Compare, FIGS. 16A and 15A, respectively. Strikingly, the presence of two Fv domains permitted necroptosis induction by a version of RIPK3 with either inactivating mutations (data not shown) or even complete deletion of the RHIM domain. See FIG. 12A and FIG. 15B. Cells expressing this RHIM-deficient RIPK3^{ΔC}-2xFv construct were nonresponsive to TNF+zVAD, previously reported to be dependent on RIPK1 RHIM/RIPK3 RHIM interactions. See, FIG. 13G. Furthermore, addition of zVAD or Nec1 to cells expressing RIPK3-2xFv or RIPK3^{ΔC}-2xFv did not alter magnitude or kinetics of cell death. See, FIG. 16C and FIG. 16D, respectively. Consistent with a conclusion of chemically-induced oligomerization, large molecular weight RIPK3 complexes were observed when cells expressing RIPK3-2xFv or RIPK3^{ΔC}-2xFv were exposed to AP1. See, FIG. 16E. Furthermore, immunoprecipitation of RIPK3-2xFv following AP1 addition revealed interaction with RIPK1 and caspase-8, regardless of the presence of zVAD or Nec1. See, FIG. 16F. These data indicate that while RIPK1 and caspase-8 are recruited to the RIPK3 oligomers formed by this construct, chemically-enforced oligomerization of RIPK3 eliminates the ability of RIPK1 or caspase-8 to modulate RIPK3 activation. In conclusion, it appears that oligomerization of the RIPK3 kinase domain is both necessary and sufficient to trigger necroptosis, irrespective of the presence of the RHIM domain. Further, the inability of inhibitors of caspase-8 and RIPK1 to modulate cell death triggered by RIPK3 oligomerization indicates that intrinsic control of this complex by caspase-8 and RIPK1 acts at, or upstream of, the formation of RIPK3 oligomers.

### IX. RIPK1 Protein Inhibits RHIM-Dependent RIPK3 Oligomerization

The above discussed data shows that “seeding” RHIM-dependent oligomers of RIPK3 via dimerization is inhibited by necrostatin 1 (Nec1). See, FIG. 15C. These data imply that RIPK1 kinase activity drives the formation of RIPK3 oligomers, and that the recruitment of RIPK1 to RIPK3 dimers similarly suggests that RIPK1 may play a role in promoting the propagation of RIPK3 oligomers.

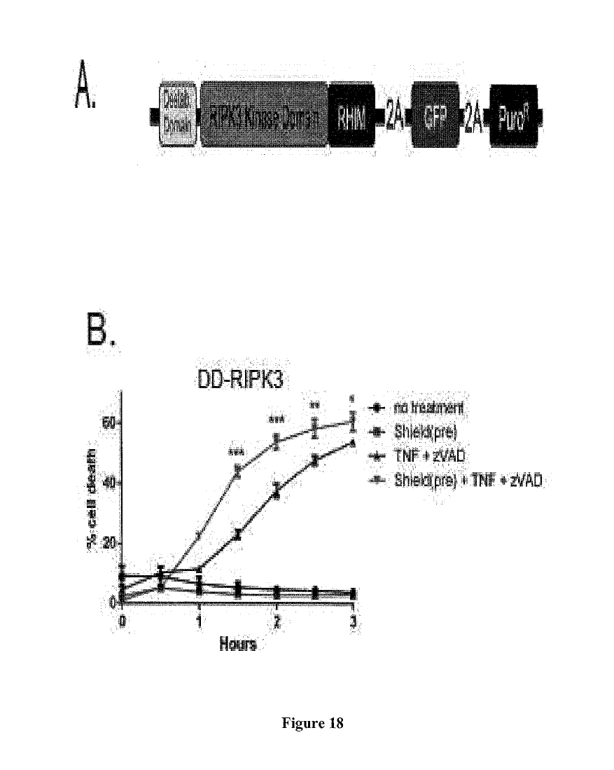

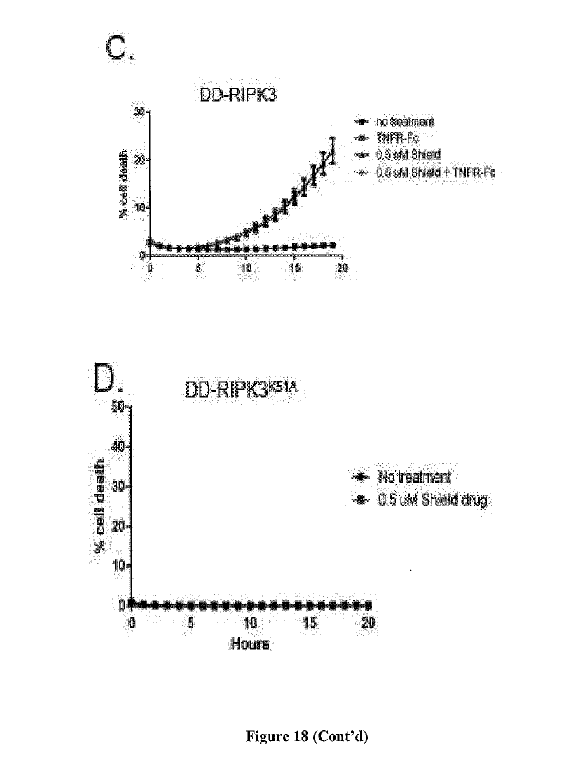

The Nec1 inhibitory effects were further investigated by knocking down RIPK1 using siRNA. See, FIG. 14A. Unexpectedly, and surprisingly, it was found that while Nec1-inhibition of RIPK1 blocked RIPK3 activation, RIPK1 knockdown significantly enhanced RIPK3 activation. See, FIG. 17A. This finding implied that the presence of the RIPK1 protein inhibits receptor-independent oligomerization of RIPK3, and that the inhibitory effects of Nec1 may rely on the scaffolding function of the chemically-inhibited RIPK1 protein. Consistent with this idea, and with an on-target effect of Nec1, that the inhibitory effects of Nec1 on RIPK3 oligomerization were observed to be abrogated upon knockdown of RIPK1. Compare, FIG. 17A and FIG. 15C, respectively. These data imply that while the kinase activity of RIPK1 can potentiate RIPK3 oligomerization, RIPK1 may also be required to exert intrinsic control of RIPK3 activation in the cytosol. Consequently, cells lacking RIPK1 should display reduced sensitivity to TNF-induced RIPK3 activation but increased sensitivity to spontaneous activation of RIPK3. RIPK3 was fused to a destabilization domain (DD) (33a), creating a version of RIPK3 that is rapidly and constitutively degraded, but that accumulates in response to a DD-binding drug, referred to as Shield. See, FIG. 18A. The data confirmed that the DD-RIPK3 fusion protein accumulated in response to Shield administration. See, FIG. 17B. Further, Shield pre-treatment increased the sensitivity of NIH-3T3 cells expressing the RIPK3-DD construct to TNF-induced necroptosis. See, FIG. 18B. RIPK3 accumulation was also sufficient to trigger spontaneous necrosome formation and limited cell death in the absence of exogenous TNF. Consistent with RIPK3 accumulation triggering spontaneous necrosome formation, this cell death was unaffected by the TNF-blocking reagent TNFR1-Fc, and could be abrogated by the K51A insertion mutation of the active site of DD-RIPK3. See, FIG. 18C and FIG. 18D, respectively.

RIPK1 was evaluated as a potential intrinsic inhibitor of RIPK3 activation in the absence of receptor signaling. Consistent with canonical roles of caspase-8 and RIPK1 following TNFR1 ligation, knockdown of caspase-8 greatly sensitized cells expressing low levels of RIPK3 to TNF-induced cell death, while knockdown of RIPK1 reduced cell death in these conditions. See, FIG. 17C. However, when either caspase-8 or RIPK1 expression were silenced in the presence of Shield drug, the stabilization of RIPK3 was sufficient to support spontaneous, TNF-independent activation of DD-RIPK3. See, FIG. 17D. Addition of the RIPK1 inhibitor Nec1 to DD-RIPK3 expressing cells decreased cell death triggered by RIPK3 accumulation, and this effect was eliminated by RIPK1 siRNA-mediated knockdown, demonstrating that the effects of Nec1 are on-target and depend on the presence of the RIPK1 protein. See, FIG. 17E. These data indicate that while RIPK1 can drive receptor-induced RIPK3 activation and necroptosis, it also acts as an intrinsic suppressor of RIPK3 necrosome formation in the absence of receptor signaling. Furthermore, the RIPK3 inhibitor Nec1 potentiates this inhibitory function by creating an inactive form of RIPK1. This indicates that genetic elimination and chemical inhibition of RIPK1 thereby have opposing effects.

The data generated herein has clear implications for RIPK3 signaling under physiological conditions. Consistent with structural studies of the RHIM domains, as well as other amyloid-forming proteins, it is likely that the RHIM domains of RIPK3 proteins in the cytosol of healthy cells are somewhat “sticky,” undergoing limited interactions in the absence of exogenous signals. Because of the self-propagating nature of these structures, these cells may have mechanisms to limit these interactions in the absence of pro-death signaling. Recruitment of the RIPK1 RHIM domain to RIPK3 oligomers, in the absence of other signals, may thereby recruit suppressive proteins to limit oligomer propagation. Consistent with this idea, cells in which RIPK1 were depleted were found resistant to TNF-induced necroptosis when RIPK3 levels were low, but underwent increased spontaneous RIPK3-dependent death when RIPK3 levels were increased.

The data presented herein also indicate that the presence of RIPK1 is a determinant of a cell's ability to tolerate increased RIPK3 levels, and raise the possibility that post natal lethality observed in RIPK1 knockouts may be partially due to unrestrained—and possibly receptor independent—RIPK3 activation. Similarly, there are other implications for pathways such as TRIF and DAI-dependent signaling in which the signaling molecules themselves contain RHIM domains. These molecules can activate RIPK3 independently of RIPK1, and indeed, RIPK1 may act to modify or inhibit RIPK3 signaling in these cases, in a manner analogous to that observed in our dimerization-based system.

Because the data also shows that siRNA knockout depletion and chemical inhibition of RIPK1 had opposing effects on RIPK3 activation, systemic use of RIPK1 inhibitors is likely to yield very different results than murine models in which RIPK1 is genetically ablated.

### X. Pharmaceutical Compositions

The present invention further provides pharmaceutical compositions (e.g., comprising the compounds and/or proteins as described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

### EXPERIMENTAL

In the examples below, the following compounds were used: Recombinant murine TNF (Peprotech 315-01A) was used at 1 ng/ml unless otherwise specified; AP1 (Now commercialized by Clontech as “B/B Homodimerizer,” catalog #635059) was dissolved in ethanol to a concentration of 100 μM, then diluted in culture media to a final concentration of 30 nM unless otherwise indicated. zVAD (SM 13 Biochemicals SMFMK001) was dissolved in DMSO to a concentration of 50 mM, then diluted to 50 μM in culture media; Necrostatin-1(Sigma N9037-10MG) was dissolved in DMSO to a concentration of 30 mM, then diluted to 30 μM in culture media; TNFR-Fc (Fisher 430-RI-050) was used at a final concentration of 200 ng/ml; Shield drug was a kind gift of Dr. Tom Wandless and was used at a final concentration of 1 μM. SiGenome SMARTpool siRNAs were purchased from Dharmacon/Fisher. Pools targeting murine MLKL(M-061420-01), murine RIPK1 (M-040150-01), and murine caspase-8 (M-043044-01), as well as a non-targeting “scramble” pool(D-001206-14), were used. Two microliters of a 50 μM stock of these siRNAs were delivered to cells in 6-well format using Lipofectamine siRNA-Max reagent (Life Technologies 13778150) according to the manufacturer's instructions. Forty-eight hours later, these cells were re-plated into 24-well format for cell death assays, or harvested for western blot analysis of knockdowns.

### Example I

### Vertebrate Animals

This example identifies the strains of mice used herein as an experimental system. Although many of the studies proposed herein are performed in cell lines, other studies require the use of primary cells from animals as well as developmental and tumor studies that can only be performed in animals. The following are examples of transgenic and knockout mice that were used for the experiments described herein.

TABLE 1
Mouse genotypes used in the proposed research

| Line/Cross | Status |
|---|---|
| Casp8^{-/-} RIPK3 DKO | In house, breeding |
| FADD^{-/-} RIPK3 DKO | In house, breeding |
| FLIP^{+/-}RIPK3^{-/-} × FLIP^{+/-}RIPK3^{-/-} | Ongoing cross for study |
| FADD FLIP RIPK3 TKO | In house, breeding |
| Tie2-Cre × Rosa-LSL-eYFP | In house, breeding |
| Tie2-Cre × casp8fl/fl | In house, breeding |
| Tie2-Cre × casp8^{fl/fl} RIPK3^{-/-} | Planned |
| Tie2-Cre × casp8^{fl/-} × LSL-YFP | In progress |
| FLIPfl/fl | In house, Breeding |
| Tie2-Cre × FLIP^{fl/fl} × RIPK3 | Planned |
| TNFR1^{-/-}casp8^{+/-} × TNFR1^{-/-}casp8^{+/-} | Ongoing cross for study |
| CYLDfl/fl | In house, breeding |
| CYLD^{fl/+} × CMV-Cre (deleter strain) | In house, breeding (for |
| | production of CYLD^{-/-}) |
| CYLD^{-/-} casp8^{+/-} × CYLD^{-/-} casp8^{+/-} | Planned |
| Casp8^{fl/fl} CD19-Cre | In house, breeding |
| Casp8^{fl/fl} Albumin-Cre | In house, breeding |
| Rag2^{-/-} | In house, breeding |

As the animals are being bred all ages and sexes are maintained in the facility. For experiments animals of both sexes aged 4-6 weeks are used in most cases. Genotyping for genetic experiments is performed in embryos, at P6, or at weaning.

### Example II

### Constructs and Cell Lines

RIPK3-Fv chimeric proteins were constructed by cloning full-length murine RIPK3, catalytically dead RIPK3^{K51A}, RIPK3 lacking the final 42 amino acids and including the RHIM domain (RIPK3^{ΔC}), or RIPK3 bearing a 4 amino acid substitution in a RHIM motif, for example VQIG→AAAA, (RIPK3^{ΔRHIM}) upstream of either one or two copies of FKBP carrying the F36V mutation, herein called “Fv” domains. One of skill in the art would understand that analogous constructs can be made from human nucleotide sequences where, for example, a human RIPK3^{ΔC} would lack the final 61 amino acids including the RHIM domain. When two Fv domains were used, the first copy contained silent mutations to prevent DNA recombination.

These RIPK3-Fv fusion proteins were cloned into pBabe-Puro retroviral vectors containing T2A ribosome-skipping sequences derived from porcine teschovirus-1 (EGRGSLLTCGDVEENPGP) upstream of eGFP, creating bicistronic constructs in which RIPK3-Fv and GFP are translated from the same mRNA, but separate upon translation to generate distinct proteins. FLAG tags were added to the N-terminus of RIPK3 during this cloning step. The resulting constructs therefore take the general form FLAG-RIPK3-Fv-T2AGFP in pBabe-Puro vectors, and the expressed RIPK3 fusion proteins, once separated from GFP, contain both an N-terminal FLAG and a C-terminal 2A epitope.

These constructs were transduced into NIH-3T3 cells using standard protocols for helper-dependent retroviral transduction. Transduced cells were selected for 5 days in 1 μg/ml puromycin, then grown to confluence and sorted twice for homogenous GFP expression. A minimum of two distinct, separately-derived stable cell lines expressing each of these proteins was generated, and experiments presented are representative of results obtained with both cell lines. All cell lines were maintained in D-MEM (Fisher, SH30022FS) supplemented with 10% FCS (Sigma, 0926-500ML), 29.2 g/L glutamine (Fisher, SH3003402), 10,000 U/mL penicillin and 10,000 μg/mL streptomycin (Fisher, SV30010), and grown at 37 degrees in 5% CO₂.

Chimeric proteins composed of RIPK3 fused to the destabilization domain (DD)(31a) were created via recombinant PCR, to produce a fragment composed of an N-terminal FLAG tag followed by the destabilization domain, then full-length murine RIPK3 or RIPK3^{K51A}. These constructs were cloned into the pRRL lentiviral vector downstream of an MND promoter, and upstream of a T2A-GFP-T2A-Puromycin resistance cassette. Thus, cells transduced with these vectors produce FLAG-DD-RIPK3, GFP, and the puromycin resistance marker as separate proteins from a single mRNA, and both DD-RIPK3 and GFP carry a C terminal 2A epitope. These constructs were expressed in NIH-3T3 cells via standard helper12 dependent lentiviral transduction, and resulting cells were selected in 2 μg/ml puromycin. Stable expression was confirmed by flow cytometric analysis. “Jax” cells are a line of SV40 immortalized C57B1/6 murine embryonic fibroblasts produced by the Jackson Laboratory. These cells express endogenous RIPK3 and undergo rapid necroptosis in response to TNF+zVAD treatment. These cells were a kind gift of Dr. Dan Stetson.

### Example III

### Cell Death Assays

Cell death assays were carried out using a 2-color IncuCyte Zoom in-incubator imaging system (Essen Biosciences.) Briefly, this system allows fully automated imaging of cells at set intervals in phase contrast as well as both red and green fluorescent channels. Cell death assays were carried out by treating cells with death-inducing compounds in 24 well tissue culture vessels (100,000 cells/well), in the presence of 100 nM of the cell-impermeable DNA binding fluorescent dyes Sytox Green (Life Technologies 57020) or Yoyo-3 (Y3606), which are excluded from healthy cells but rapidly enter dying cells upon membrane permeabilization, in a manner analogous to propidium iodide.

Resulting images were analyzed using the software package supplied with the IncuCyte imager, which allows precise analysis of the number of Sytox Green or Yoyo-3 positive cells present in each image. For each experiment, a minimum of three separate wells were treated with each experimental condition, and a minimum of 4 image fields were assessed per well. Percent cell death was calculated by treating a minimum of three distinct wells in each experiment with 100 nM of the cell permeable fluorescent dye Syto Green (S7559), which allows quantification of the total number of cells present in each field. Dead cell events acquired via Sytox Green or Yoyo-3 staining were divided by this total cell number to yield percent cell death at each timepoint. Error bars represent standard deviation from the mean of a minimum of three independent wells. Each result depicted is representative of at least 4 distinct experiments, each of which contained at least 3 technical replicates.

### Example IV

### Antibodies and Immunoprecipitation

Anti-caspase-8 (Enzo, 1G12, ALX-804-447-C100), anti-RIPK1 (BD 610458), anti-RIPK3 (Imgenex IMG-5523-2), rabbit anti-FLAG (Abeam AB1162), anti-actin (Millipore MAB1501) were used in the immunoprecipitation experiments. The anti-2A antibody was a kind gift from Dr. Dario Vignali. The anti-MLKL antibody was a kind gift from. Dr. Warren Alexander (35a).

Secondary antibodies were purchased from Santa Cruz Biotechnology (mouse sc-2005, rat sc-2006, rabbit sc-2313). These antibodies were used for Western Blots of proteins separated using SDS-PAGE pre-cast gels (Invitrogen) and transferred to PVDF membranes. Membranes were incubated with primary and HRP-conjugated secondary antibodies in TBS-T buffer containing 5% non-fat milk, then detected using ECL reagents (Pierce). Detection was accomplished using either standard autoradiography film (Pierce) or an Chemidock electronic luminescence detection platform (ChemiDoc™ XRS+ System, #170-8265).

Immunoprecipitation of necrosome complexes were carried out using rabbit anti-FLAG antibodies, and TruBlot IP reagents (Rockland Immunochemicals) to eliminate non-specific signals from immunoglobulin heavy and light chains. Briefly, cell lines were treated as indicated and lysed in NP40 buffer (30 mM Tris, 150 mM NaCl, and 1% NP40) supplemented with protease inhibitors. Twenty micrograms of each sample were reserved for “input,” while 500 μg of total protein from each sample were immunoprecipitated with 0.5 μg antibody conjugated to 30 μL TruBlot anti-Rabbit IgG beads Immunocomplexes were washed 4 times in NP40 buffer, eluted by boiling, then run on western blot. Western blots were analyzed using standard primary antibodies, but TruBlot anti-Rabbit and anti-Mouse HRP-conjugated 14 secondary antibodies were used. A standard anti-Rat-HRP secondary was used to detect caspase-8.

### Example V

### Disuccinimidyl Suberate (DSS) Crosslinking

A confluent monolayer of the indicated cells was incubated with the appropriate treatment for 30 minutes at 37 degrees. Cells were lysed in a modified, Tris-free NP40 buffer (30 mM HEPES pH 7.4, 150 mM NaCl, and 1% NP40) without protease inhibitors. Protein lysate was quantified according to standard BCA assay (Pierce) and fifty micrograms of each sample was treated with 0.1 mM DSS crosslinking agent (Thermo Scientific, #21658) for 30 minutes at room temperature; the reaction was quenched by the addition of Tris-HCl pH 7.2 to a final concentration of 50 mM for 15 minutes. Crosslinked samples were analyzed by Western blot as described above, using the anti-2A primary antibody for detection.

Particular embodiments of the invention are described above in the Summary and Detailed Description sections. Although the invention has been described in connection with specific embodiments, it should be understood that the invention as claimed should not be unduly limited to such specific embodiments. For example, the compositions and methods of the present invention are described in connection with particular homodimers, heterodimers and oligomers capable of inducing necrosis in a cell. The invention finds use with a broad array of in vivo and in vitro applications, both clinical and research related.

### REFERENCE LIST 1

1. Green, D. R. 2011. Means to an End: Apoptosis and Other Cell Death Mechanisms. Cold Spring Harbor Press, Cold Spring Harbor.
2. Varfolomeev, E. E., Schuchmann, M., Luria, V., Chiannilkulchai, N., Beckmann, J. S., Mett, I. L., Rebrikov, D., Brodianski, V. M., Kemper, O. C., Kollet, O., Lapidot, T., Soffer, D., Sobe, T., Avraham, K. B., Goncharov, T., Holtmann, H., Lonai, P., and Wallach, D. 1998. Targeted disruption of the mouse Caspase 8 gene ablates cell death induction by the TNF receptors, Fas/Apo1, and DR3 and is lethal prenatally. Immunity 9, 267-276. 9729047
3. Yeh, W. C., Pompa, J. L., McCurrach, M. E., Shu, H. B., Elia, A. J., Shahinian, A., Ng, M., Wakeham, A., Khoo, W., Mitchell, K., El-Deiry, W. S., Lowe, S. W., Goeddel, D. V., and Mak, T. W. 1998. FADD: essential for embryo development and signaling from some, but not all, inducers of apoptosis. Science 279, 1954-1958. 9506948
4. Zhang, J., Cado, D., Chen, A., Kabra, N. H., and Winoto, A. 1998. Fas-mediated apoptosis and activation-induced T-cell proliferation are defective in mice lacking FADD/Mort1. Nature 392, 296-300. 9521326
5. Kabra, N. H., Kang, C., Hsing, L. C., Zhang, J., and Winoto, A. 2001. T cell-specific FADD-deficient mice: FADD is required for early T cell development. Proc Natl Acad Sci USA 98, 6307-6312. 11353862
6. Salmena, L., Lemmers, B., Hakem, A., Matysiak-Zablocki, E., Murakami, K., Au, P. Y., Berry, D. M., Tamblyn, L., Shehabeldin, A., Migon, E., Wakeham, A., Bouchard, D., Yeh, W. C., McGlade, J. C., Ohashi, P. S., and Hakem, R. 2003. Essential role for caspase 8 in T-cell homeostasis and T-cellmediated immunity. Genes Dev 17, 883-895. 12654726
7. Black, S., Kadyrov, M., Kaufmann, P., Ugele, B., Emans, N., and Huppertz, B. 2004. Syncytial fusion of human trophoblast depends on caspase 8. Cell Death Differ 11, 90-98. 12970672
8. Rote, N. S., Wei, B. R., Xu, C., and Luo, L. 2010. Caspase 8 and human villous cytotrophoblast differentiation. Placenta 31, 89-96. 20080300
9. Green, D. R., Oberst, A., Dillon, C. P., Weinlich, R., and Salvesen, G. S. 2011. RIPK-dependent necrosis and its regulation by caspases: A mystery in five acts. Mol Cell in press.
10. Holler, N., Zaru, R., Micheau, O., Thorne, M., Attinger, A., Valitutti, S., Bodmer, J. L., Schneider, P., Seed, B., and Tschopp, J. 2000. Fas triggers an alternative, caspase-8-independent cell death pathway using the kinase RIP as effector molecule. Nat Immunol 1, 489-495. 11101870
11. Hitomi, J., Christofferson, D. E., Ng, A., Yao, J., Degterev, A., Xavier, R. J., and Yuan, J. 2008. Identification of a molecular signaling network that regulates a cellular necrotic cell death pathway. Cell 135, 1311-1323. 19109899
12. Degterev, A., Hitomi, J., Germscheid, M., Ch'en, I. L., Korkina, O., Teng, X., Abbott, D., Cuny, G. D., Yuan, C., Wagner, G., Hedrick, S. M., Gerber, S. A., Lugovskoy, A., and Yuan, J. 2008. Identification of RIP1 kinase as a specific cellular target of necrostatins. Nat Chem Biol 4, 313-321. 18408713
13. Zhang, D. W., Shao, J., Lin, J., Zhang, N., Lu, B. J., Lin, S. C., Dong, M. Q., and Han, J. 2009. RIP3, an energy metabolism regulator that switches TNF-induced cell death from apoptosis to necrosis. Science 325, 332-336. 19498109
14. He, S., Wang, L., Miao, L., Wang, T., Du, F., Zhao, L., and Wang, X. 2009. Receptor interacting protein kinase-3 determines cellular necrotic response to TNF-alpha. Cell 137, 1100-1111. 19524512
15. Cho, Y. S., Challa, S., Moquin, D., Genga, R., Ray, T. D., Guildford, M., and Chan, F. K. 2009. Phosphorylation-driven assembly of the RIP1-RIP3 complex regulates programmed necrosis and virusinduced inflammation. Cell 137, 1112-1123. 19524513
16. Bell, B. D., Leverrier, S., Weist, B. M., Newton, R. H., Arechiga, A. F., Luhrs, K. A., Morrissette, N. S., and Walsh, C. M. 2008. FADD and caspase-8 control the outcome of autophagic signaling in proliferating T cells. Proc Natl Acad Sci USA 105, 16677-16682. 18946037
17. Ch'en, I. L., Beisner, D. R., Degterev, A., Lynch, C., Yuan, J., Hoffmann, A., and Hedrick, S. M. 2008. Antigen-mediated T cell expansion regulated by parallel pathways of death. Proc Natl Acad Sci USA 105, 17463-17468. 18981423
18. Oberst, A., Dillon, C. P., Weinlich, R., McCormick, L. L., Fitzgerald, P., Pop, C., Hakem, R., Salvesen, G. S., and Green, D. R. 2011. Catalytic activity of the caspase-8-FLIP(L) complex inhibits RIPK3-dependent necrosis. Nature 471, 363-367. 21368763
19. Kaiser, W. J., Upton, J. W., Long, A. B., Livingston-Rosanoff, D., Daley-Bauer, L. P., Hakem, R., Caspary, and Mocarski, E. S. 2011. RIP3 mediates the embryonic lethality of caspase-8-deficient mice. Nature 471, 368-372. 21368762
20. Zhang, H., Zhou, X., McQuade, T., Li, J., Chan, F. K., and Zhang, J. 2011. Functional complementation between FADD and RIP1 in embryos and lymphocytes. Nature 471, 373-376. 21368761
21. Kelliher, M. A., Grimm, S., Ishida, Y., Kuo, F., Stanger, B. Z., and Leder, P. 1998. The death domain kinase RIP mediates the TNF-induced NF-kappaB signal. Immunity 8, 297-303. 9529147
22. Bidere, N., Su, H. C., and Lenardo, M. J. 2006. Genetic disorders of programmed cell death in the immune system. Annu Rev Immunol 24, 321-352. 16551252
23. Weinlich, R., Dillon, C. P., and Green, D. R. 2011. Ripped to death. Trends Cell Biol. in press
24. Oberst, A., and Green, D. R. 2011. It cuts both ways: reconciling the dual roles of caspase-8 in cell death and survival. Nat Rev Mol Cell Biol in press.
25. Upton, J. W., Kaiser, W. J., and Mocarski, E. S. 2010. Virus inhibition of RIP3-dependent necrosis. Cell Host Microbe 7, 302-313. 20413098
26. Feoktistova, M., Geserick, P., Kellert, B., Dimitrova, D. P., Langlais, C., Hupe, M., Cain, K., MacFarlane, M., Hacker, G., and Leverkus, M. 2011. cIAPs block Ripoptosome formation, a RIP1/caspase-8 containing intracellular cell death complex differentially regulated by cFLIP isoforms. Mol Cell 43, 449-463. 21737330
27. Oberst, A., Pop, C., Tremblay, A. G., Blais, V., Denault, Salvesen, G. S., and Green, D. R. 2011. Inducible dimerization and inducible cleavage reveal a requirement for both processes in caspase-8 activation. J Biol Chem 285, 16632-16642. 20308068
28. Pop, C., Oberst, A., Drag, M., Van Raam, B. J., Riedl, S. J., Green, D. R., and Salvesen, G. S. 2011. FLIP(L) induces caspase 8 activity in the absence of interdomain caspase 8 cleavage and alters substrate specificity. Biochem J 433, 447-457. 21235526
29. Kang, T. B., Oh, G. S., Scandella, E., Bolinger, B., Ludewig, B., Kovalenko, A., and Wallach, D. 2008. Mutation of a self-processing site in caspase-8 compromises its apoptotic but not its nonapoptotic functions in bacterial artificial chromosome-transgenic mice. J Immunol 181, 2522-2532. 18684943
30. Kang, T. B., Ben-Moshe, T., Varfolomeev, E. E., Pewzner-Jung, Y., Yogev, N., Jurewicz, A., Waisman, A., Brenner, O., Haffner, R., Gustafsson, E., Ramakrishnan, P., Lapidot, T., and Wallach, D. 2004. Caspase-8 serves both apoptotic and nonapoptotic roles. J Immunol 173, 2976-2984. 15322156
31. Sakamaki, K., Inoue, T., Asano, M., Sudo, K., Kazama, H., Sakagami, J Sakata, S., Ozaki, M., Nakamura, S., Toyokuni, S., Osumi, N., Iwakura, Y., and Yonehara, S. 2002. Ex vivo whole-embryo culture of caspase-8-deficient embryos normalize their aberrant phenotypes in the developing neural tube and heart. Cell Death Differ 9, 1196-1206. 12404118
32. Yeh, W. C., Itie, A., Elia, A. J., Ng, M., Shu, H. B., Wakeham, A., Mirtsos, C., Suzuki, N., Bonnard, M., Goeddel, D. V., and Mak, T. W. 2000. Requirement for Casper (c-FLIP) in regulation of death receptorinduced apoptosis and embryonic development. Immunity 12, 633-642. 10894163
33. Boisset, J. C., van Cappellen, W., Andrieu-Soler, C., Galjart, N., Dzierzak, E., and Robin, C. 2010. In vivo imaging of haematopoietic cells emerging from the mouse aortic endothelium. Nature 464, 116-120. 20154729
34. de Bruijn, M. F., Ma, X., Robin, C., Ottersbach, K., Sanchez, M J., and Dzierzak, E. 2002. Hematopoietic stem cells localize to the endothelial cell layer in the midgestation mouse aorta. Immunity 16, 673-683. 12049719
35. Dzierzak, E. 2005. The emergence of definitive hematopoietic stem cells in the mammal. Curr Opin Hematol 12, 197-202. 15867575
36. Dzierzak, E., and Speck, N. A. 2008. Of lineage and legacy: the development of mammalian hematopoietic stem cells. Nat Immunol 9, 129-136. 18204427
37. Sanchez, M. J., Holmes, A., Miles, C., and Dzierzak, E. 1996. Characterization of the first definitive hematopoietic stem cells in the AGM and liver of the mouse embryo. Immunity 5, 513-525. 8986712
38. Yokomizo, T., and Dzierzak, E. 2010. Three-dimensional cartography of hematopoietic clusters in the vasculature of whole mouse embryos. Development 137, 3651-3661. 20876651
39. Yokomizo, T., Ng, C. E., Osato, M., and Dzierzak, E. 2011. Three-dimensional imaging of whole midgestation murine embryos shows an intravascular localization for all hematopoietic clusters. Blood 117, 6132-6134. 21505195
40. Dumont, D. J., Gradwohl, G., Fong, G. H., Puri, M. C., Gertsenstein, M., Auerbach, A., and Breitman, M. L. 1994. Dominant-negative and targeted null mutations in the endothelial receptor tyrosine kinase, tek, reveal a critical role in vasculogenesis of the embryo. Genes Dev 8, 1897-1909. 7958865
41. Sato, T. N., Tozawa, Y., Deutsch, U., Wolburg-Buchholz, K., Fujiwara, Y., Gendron-Maguire, M., Gridley, T., Wolburg, H., Risau, W., and Qin, Y. 1995. Distinct roles of the receptor tyrosine kinases Tie-1 and Tie-2 in blood vessel formation. Nature 376, 70-74. 7596437
42. Kisanuki, Y. Y., Hammer, R. E., Miyazaki, J., Williams, S. C., Richardson, J. A., and Yanagisawa, M. 2001. Tie2-Cre transgenic mice: a new model for endothelial cell-lineage analysis in vivo. Dev Biol 230, 230-242. 11161575
43. Tang, Y., Harrington, A., Yang, X., Friesel, R. E., and Liaw, L. 2010. The contribution of the Tie2+ lineage to primitive and definitive hematopoietic cells. Genesis 48, 563-567. 20645309
44. North, T. E., de Bruijn, M. F., Stacy, T., Talebian, L., Lind, E., Robin, C., Binder, M., Dzierzak, E., and Speck, N. A. 2002. Run×1 expression marks long-term repopulating hematopoietic stem cells in the midgestation mouse embryo. Immunity 16, 661-672. 12049718
45. Srinivasan, A., Roth, K. A., Sayers, R. O., Shindler, K. S., Wong, A. M., Fritz, L. C., and Tomaselli, K. J. 1998. In situ immunodetection of activated caspase-3 in apoptotic neurons in the developing nervous system. Cell Death Differ 5, 1004-1016. 9894607
46. Kovalenko, A., Kim, J. C., Kang, T. B., Rajput, A., Bogdanov, K., Dittrich-Breiholz, O., Kracht, M., Brenner, O., and Wallach, D. 2009. Caspase-8 deficiency in epidermal keratinocytes triggers an inflammatory skin disease. J Exp Med 206, 2161-2177. 19720838
47. Gunther, C., Martini, E., Wittkopf, N., Amann, K., Weigmann, B., Neumann, H., Waldner, M. J., Hedrick, S. M., Tenzer, S., Neurath, M. F., and Becker, C. 2011. Caspase-8 regulates TNF-alpha-induced epithelial necroptosis and terminal ileitis. Nature 477, 335-339. 21921917
48. Beisner, D. R., Ch'en, I. L., Kolla, R. V., Hoffmann, A., and Hedrick, S. M. 2005. Cutting edge: innate immunity conferred by B cells is regulated by caspase-8. J Immunol 175, 3469-3473. 16148088
49. Newton, K., Sun, X., and Dixit, V. M. 2004. Kinase RIP3 is dispensable for normal NF-kappa Bs, signaling by the B-cell and T-cell receptors, tumor necrosis factor receptor 1, and Toll-like receptors 2 and 4. Mol Cell Biol 24, 1464-1469. 14749364
50. Tenev, T., Bianchi, K., Darding, M., Broemer, M., Langlais, C., Wallberg, F., Zachariou, A., Lopez, J., MacFarlane, M., Cain, K., and Meier, P. 2011. The Ripoptosome, a signaling platform that assembles in response to genotoxic stress and loss of IAPs. Mol Cell 43, 432-448. 21737329
51. Ch'en, I. L., Tsau, J. S., Molkentin, J. D., Komatsu, M., and Hedrick, S. M. 2011. Mechanisms of necroptosis in T cells. J Exp Med 208, 633-641. 21402742
52. Welz, P. S., Wullaert, A., Vlantis, K., Kondylis, V., Fernandez-Majada, V., Ermolaeva, M., Kirsch, P., Sterner-Kock, A., van Loo, G., and Pasparakis, M. 2011. FADD prevents RIP3-mediated epithelial cell necrosis and chronic intestinal inflammation. Nature 477, 330-334. 21804564
53. O'Donnell, M. A., Perez-Jimenez, E., Oberst, A., Ng, A., Massoumi, R., Xavier, R., Green, D. R., and Ting, A. T. 2011. Caspase-8 inhibits programmed necrosis by processing CYLD. Nat. Cell Biol. in press
54. Wang, L., Du, F., and Wang, X. 2008. TNF-alpha induces two distinct caspase-8 activation pathways. Cell 133, 693-703. 18485876
55. Reiley, W. W., Zhang, M., Jin, W., Losiewicz, M., Donohue, K. B., Norbury, C. C., and Sun, S. C. 2006. Regulation of T cell development by the deubiquitinating enzyme CYLD. Nat Immunol 7, 411-417. 16501569
56. Massoumi, R., Chmielarska, K., Hennecke, K., Pfeifer, A., and Fassler, R. 2006. Cyld inhibits tumor cell proliferation by blocking Bcl-3-dependent NF-kappaB signaling. Cell 125, 665-677. 16713561
57. Sun, S. C. 2010. CYLD: a tumor suppressor deubiquitinase regulating NF-kappaB activation and diverse biological processes. Cell Death Differ 17, 25-34. 19373246
58. Medvinsky, A., Rybtsov, S., and Taoudi, S. 2011. Embryonic origin of the adult hematopoietic system: advances and questions. Development 138, 1017-1031. 21343360
59. Sun, T., Gao, Y., Tan, W., Ma, S., Shi, Y., Yao, J., Guo, Y., Yang, M., Zhang, X., Zhang, Q., Zeng, C., and Lin, D. 2007. A six-nucleotide insertion-deletion polymorphism in the CASP8 promoter is associated with susceptibility to multiple cancers. Nat Genet 39, 605-613. 17450141
60. Li, C., Lu, J., Liu, Z., Wang, L. E., Zhao, H., El-Naggar, A. K., Sturgis, E. M., and Wei, Q. 2011. The sixnucleotide deletion/insertion variant in the CASP8 promoter region is inversely associated with risk of squamous cell carcinoma of the head and neck. Cancer Prev Res (Phila) 3, 246-253. 20086182
61. Teitz, T., Wei, T., Valentine, M. B., Vanin, E. F., Grenet, J., Valentine, V. A., Behm, F. G., Look, A. T., Lahti, J. M., and Kidd, V. J. 2000. Caspase 8 is deleted or silenced preferentially in childhood neuroblastomas with amplification of MYCN. Nat Med 6, 529-535. 10802708
62. Shivapurkar, N., Toyooka, S., Eby, M. T., Huang, C. X., Sathyanarayana, U. G., Cunningham, H. T., Reddy, J. L., Brambilla, E., Takahashi, T., Minna, J. D., Chaudhary, P. M., and Gazdar, A. F. 2002. Differential inactivation of caspase-8 in lung cancers. Cancer Biol Ther 1, 65-69. 12170765
63. Pingoud-Meier, C., Lang, D., Janss, A. J., Rorke, L. B., Phillips, P. C., Shalaby, T., and Grotzer, M. A. 2003. Loss of caspase-8 protein expression correlates with unfavorable survival outcome in childhood medulloblastoma. Clin Cancer Res 9, 6401-6409. 14695141
64. Kim, H. S., Lee, J. W., Soung, Y. H., Park, W. S., Kim, S. Y., Lee, J. H., Park, J. Y., Cho, Y. G., Kim, C. J., Jeong, S. W., Nam, S. W., Kim, S. H., Lee, J. Y., Yoo, N. J., and Lee, S. H. 2003. Inactivating mutations of caspase-8 gene in colorectal carcinomas. Gastroenterology 125, 708-715. 12949717
65. Qi, L., Bellail, A. C., Rossi, M. R., Zhang, Z., Pang, H., Hunter, S., Cohen, C., Moreno, C. S., Olson, J. J., Li, S., and Hao, C. 2011. Heterogeneity of primary glioblastoma cells in the expression of caspase-8 and the response to TRAIL-induced apoptosis. Apoptosis 21877214
66. Krelin, Y., Zhang, L., Kang, T. B., Appel, E., Kovalenko, A., and Wallach, D. 2008. Caspase-8 deficiency facilitates cellular transformation in vitro. Cell Death Differ 15, 1350-1355. 18566604
67. Gerlach, B., Cordier, S. M., Schmukle, A. C., Emmerich, C. H., Rieser, E., Haas, T. L., Webb, A. I., Rickard, J. A., Anderton, H., Wong, W. W., Nachbur, U., Gangoda, L., Warnken, U., Purcell, A. W., Silke, J., and Walczak, H. 2011. Linear ubiquitination prevents inflammation and regulates immune signalling. Nature 471, 591-596. 21455173
68. Shembade, N., Ma, A., and Harhaj, E. W. 2010. Inhibition of NF-kappaB signaling by A20 through disruption of ubiquitin enzyme complexes. Science 327, 1135-1139. 20185725
69. Vandenabeele, P., Declercq, W., Van Herreweghe, F., and Vanden Berghe, T. 2010. The role of the kinases RIP1 and RIP3 in TNF-induced necrosis. Sci Signal 3, re4. 20354226
70. Annunziata, C. M., Davis, R. E., Demchenko, Y., Bellamy, W., Gabrea, A., Zhan, F., Lenz, G., Hanamura, I., Wright, G., Xiao, W., Dave, S., Hurt, E. M., Tan, B., Zhao, H., Stephens, O., Santra, M., Williams, D. R., Dang, L., Barlogie, B., Shaughnessy, J. D., Jr., Kuehl, W. M., and Staudt, L. M. 2007. Frequent engagement of the classical and alternative NF-kappaB pathways by diverse genetic abnormalities in multiple myeloma. Cancer Cell 12, 115-130. 17692804
71. Schmidt, A., Schmitz, R., Giefing, M., Martin-Subero, J. I., Gesk, S., Vater, I., Massow, A., Maggio, E., Schneider, M., Hansmann, M. L., Siebert, R., and Kuppers, R. 2010. Rare occurrence of biallelic CYLD gene mutations in classical Hodgkin lymphoma. Genes Chromosomes Cancer 49, 803-809. 20607853
72. Strobel, P., Zettl, A., Ren, Z., Starostik, P., Riedmiller, H., Storkel, S., Muller-Hermelink, H. K., and Marx, A. 2002. Spiradenocylindroma of the kidney: clinical and genetic findings suggesting a role of somatic mutation of the CYLD1 gene in the oncogenesis of an unusual renal neoplasm. Am J Surg Pathol 26, 119-124. 11756779
73. Hashimoto, K., Mori, N., Tamesa, T., Okada, T., Kawauchi, S., Oga, A., Furuya, T., Tangoku, A., Oka, M., and Sasaki, K. 2004. Analysis of DNA copy number aberrations in hepatitis C virus-associated hepatocellular carcinomas by conventional CGH and array CGH. Mod Pathol 17, 617-622. 15133472
74. Hirai, Y., Kawamata, Y., Takeshima, N., Furuta, R., Kitagawa, T., Kawaguchi, T., Hasumi, K., Sugai, S., and Noda, T. 2004. Conventional and array-based comparative genomic hybridization analyses of novel cell lines harboring HPV18 from glassy cell carcinoma of the uterine cervix. Int J Oncol 24, 977-986. 15010838
75. Zhang, J., Stirling, B., Temmerman, S. T., Ma, C. A., Fuss, I. J., Deny, J. M., and Jain, A. 2006. Impaired regulation of NF-kappaB and increased susceptibility to colitis-associated tumorigenesis in CYLDdeficient mice. J Clin Invest 116, 3042-3049. 17053834
76. Jin, W., Reiley, W. R., Lee, A. J., Wright, A., Wu, X., Zhang, M., and Sun, S. C. 2007. Deubiquitinating enzyme CYLD regulates the peripheral development and naive phenotype maintenance of B cells. J Biol Chem 282, 15884-15893. 17392286
77. Hovelmeyer, N., Wunderlich, F. T., Massoumi, R., Jakobsen, C. G., Song, J., Worns, M. A., Merkwirth, C., Kovalenko, A., Aumailley, M., Strand, D., Bruning, J. C., Galle, P. R., Wallach, D., Fassler, R., and Waisman, A. 2007. Regulation of B cell homeostasis and activation by the tumor suppressor gene CYLD. J Exp Med 204, 2615-2627. 17923499
78. Stegmeier, F., Sowa, M. E., Nalepa, G., Gygi, S. P., Harper, J. W., and Elledge, S. J. 2007. The tumor suppressor CYLD regulates entry into mitosis. Proc Natl Acad Sci USA 104, 8869-8874. 17495026
79. Martinez, J., Almendinger, J., Oberst, A., Ness, R., Dillon, C. P., Fitzgerald, P., Hengartner, M. O., and Green, D. R. 2011. Microtubule-associated protein 1 light chain 3 alpha (LC3)-associated phagocytosis is required for the efficient clearance of dead cells. Proc. Natl. Acad. Sci. USA in press.
80. Williams, R. T., Roussel, M. F., and Sherr, C. J. 2006. Arf gene loss enhances oncogenicity and limits imatinib response in mouse models of Bcr-Abl-induced acute lymphoblastic leukemia. Proc Natl Acad Sci USA 103, 6688-6693. 16618932
81. Hutti, J. E., Shen, R. R., Abbott, D. W., Zhou, A. Y., Sprott, K. M., Asara, J. M., Hahn, W. C., and Cantley, L. C. 2009. Phosphorylation of the tumor suppressor CYLD by the breast cancer oncogene IKKepsilon promotes cell transformation. Mol Cell 34, 461-472. 19481526
82. Chun, H. J., Zheng, L., Ahmad, M., Wang, J., Speirs, C. K., Siegel, R. M., Dale, J. K., Puck, J., Davis, J., Hall, C. G., Skoda-Smith, S., Atkinson, T. P., Straus, S. E., and Lenardo, M. J. 2002. Pleiotropic defects in lymphocyte activation caused by caspase-8 mutations lead to human immunodeficiency. Nature 419, 395-399. 12353035
83. Blanchard, H., Kodandapani, L., Mittl, P. R., Marco, S. D., Krebs, J. F., Wu, J. C., Tomaselli, K. J., and Grutter, M. G. 1999. The three-dimensional structure of caspase-8: an initiator enzyme in apoptosis. Structure 7, 1125-1133. 10508784
84. Adams, J. M., Harris, A. W., Pinkert, C. A., Corcoran, L. M., Alexander, W. S., Cory, S., Palmiter, R. D., and Brinster, R. L. 1985. The c-myc oncogene driven by immunoglobulin enhancers induces lymphoid malignancy in transgenic mice. Nature 318, 533-538. 3906410
85. Wallach, D., Kovalenko, A., and Kang, T. B. 2011. ‘Necrosome’-induced inflammation: must cells die for it? Trends Immunol 21890409
86. Green, D. R., Ferguson, T., Zitvogel, L., and Kroemer, G. 2009. Immunogenic and tolerogenic cell death. Nat Rev Immunol 9, 353-363. 19365408
87. Amara, J. F., Clackson, T., Rivera, V. M., Guo, T., Keenan, T., Natesan, S., Pollock, R., Yang, W., Courage, N. L., Holt, D. A., and Gilman, M. 1997. A versatile synthetic dimerizer for the regulation of protein-protein interactions. Proc Natl Acad Sci USA 94, 10618-10623. 9380684
88. Straathof, K. C., Pule, M. A., Yotnda, P., Dotti, G., Vanin, E. F., Brenner, M. K., Heslop, H. E., Spencer, D. M., and Rooney, C. M. 2005. An inducible caspase 9 safety switch for T-cell therapy. Blood 105, 4247-4254. 15728125
89. Burnett, S. H., Beus, B. J., Avdiushko, R., Qualls, J., Kaplan, A. M., and Cohen, D. A. 2006. Development of peritoneal adhesions in macrophage depleted mice. J Surg Res 131, 296-301. 16289593
90. Corcoran, L. M., Tawfilis, S., and Barlow, L. J. 1999. Generation of B lymphoma cell lines from knockout mice by transformation in vivo with an Emu-myc transgene. J Immunol Methods 228, 131-138. 10556550
91. Trauth, B. C., Klas, C., Peters, A. M., Matzku, S., Moller, P., Falk, W., Debatin, K. M., and Krammer, P. H. 1989. Monoclonal antibody-mediated tumor regression by induction of apoptosis. Science 245, 301-305. 2787530
92. Ogasawara, J., Watanabe-Fukunaga, R., Adachi, M., Matsuzawa, A., Kasugai, T., Kitamura, Y., Itoh, N., Suda, T., and Nagata, S. 1993. Lethal effect of the anti-Fas antibody in mice. Nature 364, 806-809. 7689176
93. Rodriguez, I., Matsuura, K., Ody, C., Nagata, S., and Vassalli, P. 1996. Systemic injection of a tripeptide inhibits the intracellular activation of CPP32-like proteases in vivo and fully protects mice against Fas-mediated fulminant liver destruction and death. J Exp Med 184, 2067-2072. 8920897
94. Toledo, F., Krummel, K. A., Lee, C. J., Liu, C. W., Rodewald, L. W., Tang, M., and Wahl, G. M. 2006. A mouse p53 mutant lacking the proline-rich domain rescues Mdm4 deficiency and provides insight into the Mdm2-Mdm4-p53 regulatory network. Cancer Cell 9, 273-285. 16616333

### REFERENCE LIST 2

Akalu, A., and J. K. Reichardt. 2002. Increasing PCR sensitivity for amplification from paraffin-embedded tissues. Methods Mol Biol 192:67-74.
Barth, B. M., S. J. Gustafson, and T. B. Kuhn. 2011. Neutral sphingomyelinase activation precedes NADPH oxidase-dependent damage in neurons exposed to the proinflammatory cytokine tumor necrosis factor-alpha. J Neurosci Res
Bell, B. D., S. Leverrier, B. M. Weist, R. H. Newton, A. F. Arechiga, K. A. Luhrs, N. S. Morrissette, and C. M. Walsh. 2008. FADD and caspase-8 control the outcome of autophagic signaling in proliferating T cells. Proc Natl Acad Sci USA 105:16677-16682.
Bidere, N., H. C. Su, and M. J. Lenardo. 2006. Genetic disorders of programmed cell death in the immune system. Anna Rev Immunol 24:321-352.
Black, S., M. Kadyrov, P. Kaufmann, B. Ugele, N. Emans, and B. Huppertz. 2004. Syncytial fusion of human trophoblast depends on caspase 8. Cell Death Differ 11:90-98.
Cauwels, A., B. Janssen, A. Waeytens, C. Cuvelier, and P. Brouckaert. 2003. Caspase inhibition causes hyperacute tumor necrosis factor-induced shock via oxidative stress and phospholipase A2. Nat Immunol 4:387-393.
Ch'en, I. L., D. R. Beisner, A. Degterev, C. Lynch, J. Yuan, A. Hoffmann, and S. M. Hedrick. 2008. Antigen-mediated T cell expansion regulated by parallel pathways of death. Proc Natl Acad Sci USA 105:17463-17468.
Ch'en, I. L., J. S. Tsau, J. D. Molkentin, M. Komatsu, and S. M. Hedrick. 2011. Mechanisms of necroptosis in T cells. J Exp Med 208:633-641.
Cho, Y. S., S. Challa, D. Moquin, R. Genga, T. D. Ray, M. Guildford, and F. K. Chan. 2009. Phosphorylation-driven assembly of the RIP1-RIP3 complex regulates programmed necrosis and virus-induced inflammation. Cell 137:1112-1123.
Degterev, A., J. Hitomi, M. Germscheid, I. L. Ch'en, O. Korkina, X. Teng, D. Abbott, G. D. Cuny, C. Yuan, G. Wagner, S. M. Hedrick, S. A. Gerber, A. Lugovskoy, and J. Yuan. 2008. Identification of RIP1 kinase as a specific cellular target of necrostatins. Nat Chem Biol 4:313-321.
Feng, S., Y. Yang, Y. Mei, L. Ma, D. E. Zhu, N. Hoti, M. Castanares, and M. Wu. 2007. Cleavage of RIP3 inactivates its caspase-independent apoptosis pathway by removal of kinase domain. Cell Signal 19:2056-2067.
Feoktistova, M., P. Geserick, B. Kellert, D. P. Dimitrova, C. Langlais, M. Hupe, K. Cain, M. MacFarlane, G. Hacker, and M. Leverkus. 2011. cIAPs block Ripoptosome formation, a RIP1/caspase-8 containing intracellular cell death complex differentially regulated by cFLIP isoforms. Mol Cell 43:449-463.
Green, D. R. 2011. Means to an End: Apoptosis and Other Cell Death Mechanisms. Cold Spring Harbor Press, Cold Spring Harbor.
Green, D. R., A. Oberst, C. P. Dillon, R. Weinlich, and G. S. Salvesen. 2011. RIPK-dependent necrosis and its regulation by caspases: A mystery in five acts. Mol Cell in press:
Gunther, C., E. Martini, N. Wittkopf, K. Amann, B. Weigmann, H. Neumann, M. J. Waldner, S. M. Hedrick, S. Tenzer, M. F. Neurath, and C. Becker. 2011. Caspase-8 regulates TNF-alpha-induced epithelial necroptosis and terminal ileitis. Nature 477:335-339.
He, S., L. Wang, L. Miao, T. Wang, F. Du, L. Zhao, and X. Wang. 2009. Receptor interacting protein kinase-3 determines cellular necrotic response to TNF-alpha. Cell 137:1100-1111.
Hitomi, J., D. E. Christofferson, A. Ng, J. Yao, A. Degterev, R. J. Xavier, and J. Yuan. 2008. Identification of a molecular signaling network that regulates a cellular necrotic cell death pathway. Cell 135:1311-1323.
Holler, N., R. Zaru, O. Micheau, M. Thorne, A. Attinger, S. Valitutti, J. L. Bodmer, P. Schneider, B. Seed, and J. Tschopp. 2000. Fas triggers an alternative, caspase-8-independent cell death pathway using the kinase RIP as effector molecule. Nat Immunol 1:489-495.
Hughes, M. A., N. Harper, M. Butterworth, K. Cain, G. M. Cohen, and M. MacFarlane. 2009. Reconstitution of the death-inducing signaling complex reveals a substrate switch that determines CD95-mediated death or survival. Mol Cell 35:265-279.
Jost, P. J., S. Grabow, D. Gray, M. D. McKenzie, U. Nachbur, D. C. Huang, P. Bouillet, H. E. Thomas, C. Borner, J. Silke, A. Strasser, and T. Kaufmann. 2009. XIAP discriminates between type I and type II FAS-induced apoptosis. Nature 460:1035-1039.
Kabra, N. H., C. Kang, L. C. Hsing, J. Zhang, and A. Winoto. 2001. T cell-specific FADD-deficient mice: FADD is required for early T cell development. Proc Natl Acad Sci USA 98:6307-6312.
Kaiser, W. J., J. W. Upton, A. B. Long, D. Livingston-Rosanoff, L. P. Daley-Bauer, R. Hakem, T. Caspary, and E. S. Mocarski. 2011. RIPS mediates the embryonic lethality of caspase-8-deficient mice. Nature 471:368-372.
Kang, T. B., T. Ben-Moshe, E. E. Varfolomeev, Y. Pewzner-Jung, N. Yogev, A. Jurewicz, A. Waisman, O. Brenner, R. Haffner, E. Gustafsson, P. Ramakrishnan, T. Lapidot, and D. Wallach. 2004. Caspase-8 serves both apoptotic and nonapoptotic roles. J Immunol 173:2976-2984.
Kang, T. B., G. S. Oh, E. Scandella, B. Bolinger, B. Ludewig, A. Kovalenko, and D. Wallach. 2008. Mutation of a self-processing site in caspase-8 compromises its apoptotic but not its nonapoptotic functions in bacterial artificial chromosome-transgenic mice. J Immunol 181:2522-2532.
Kelliher, M. A., S. Grimm, Y. Ishida, F. Kuo, B. Z. Stanger, and P. Leder. 1998. The death domain kinase RIP mediates the TNF-induced NF-kappaB signal Immunity 8:297-303.
Kim, Y. S., M. J. Morgan, S. Choksi, and Z. G. Liu. 2007. TNF-induced activation of the Noxi NADPH oxidase and its role in the induction of necrotic cell death. Mol Cell 26:675-687.
Kovalenko, A., J. C. Kim, T. B. Kang, A. Rajput, K. Bogdanov, O. Dittrich-Breiholz, M. Kracht, O. Brenner, and D. Wallach. 2009. Caspase-8 deficiency in epidermal keratinocytes triggers an inflammatory skin disease. J Exp Med 206:2161-2177.
Li, H., H. Zhu, C. J. Xu, and J. Yuan. 1998. Cleavage of BID by caspase 8 mediates the mitochondrial damage in the Fas pathway of apoptosis. Cell 94:491-501.
Luo, X., I. Budihardjo, H. Zou, C. Slaughter, and X. Wang. 1998. Bid, a Bcl2 interacting protein, mediates cytochrome c release from mitochondria in response to activation of cell surface death receptors. Cell 94:481-490.
McStay, G. P., G. S. Salvesen, and D. R. Green. 2008. Overlapping cleavage motif selectivity of caspases: implications for analysis of apoptotic pathways. Cell Death Differ 15:322-331.
Nakagawa, T., S. Shimizu, T. Watanabe, O. Yamaguchi, K. Otsu, H. Yamagata, H. Inohara, T. Kubo, and Y. Tsujimoto. 2005. Cyclophilin D-dependent mitochondrial permeability transition regulates some necrotic but not apoptotic cell death. Nature 434:652-658.
O'Donnell, M. A., E. Perez-Jimenez, A. Oberst, A. Ng, R. Massoumi, R. Xavier, D. R. Green, and A. T. Ting. 2011. Caspase-8 inhibits programmed necrosis by processing CYLD. Nat. Cell Biol. in press:
Oberst, A., C. P. Dillon, R. Weinlich, L. L. McCormick, P. Fitzgerald, C. Pop, R. Hakem, G. S. Salvesen, and D. R. Green. 2011a. Catalytic activity of the caspase-8-FLIP(L) complex inhibits RIPK3-dependent necrosis. Nature 471:363-367.
Oberst, A., and D. R. Green. 2011. It cuts both ways: reconciling the dual roles of caspase-8 in cell death and survival. Nat Rev Mol Cell Biol in press:
Oberst, A., C. Pop, A. G. Tremblay, V. Blais, J. B. Denault, G. S. Salvesen, and D. R. Green. 2011b. Inducible dimerization and inducible cleavage reveal a requirement for both processes in caspase-8 activation. J Biol Chem 285:16632-16642.
Pop, C., A. Oberst, M. Drag, B. J. Van Raam, S. J. Riedl, D. R. Green, and G. S. Salvesen. 2011. FLIP(L) induces caspase 8 activity in the absence of interdomain caspase 8 cleavage and alters substrate specificity. Biochem J 433:447-457.
Premsrirut, P. K., L. E. Dow, S. Y. Kim, M. Camiolo, C. D. Malone, C. Miething, C. Scuoppo, J. Zuber, R. A. Dickins, S. C. Kogan, K. R. Shroyer, R. Sordella, G. J. Hannon, and S. W. Lowe. 2011. A rapid and scalable system for studying gene function in mice using conditional RNA interference. Cell 145:145-158.
Rote, N. S., B. R. Wei, C. Xu, and L. Luo. 2010. Caspase 8 and human villous cytotrophoblast differentiation. Placenta 31:89-96.
Sakamaki, K., T. Inoue, M. Asano, K. Sudo, H. Kazama, J. Sakagami, S. Sakata, M. Ozaki, S. Nakamura, S. Toyokuni, N. Osumi, Y. Iwakura, and S. Yonehara. 2002. Ex vivo whole-embryo culture of caspase-8-deficient embryos normalize their aberrant phenotypes in the developing neural tube and heart. Cell Death Differ 9:1196-1206.
Salmena, L., B. Lemmers, A. Hakem, E. Matysiak-Zablocki, K. Murakami, P. Y. Au, D. M. Berry, L. Tamblyn, A. Shehabeldin, E. Migon, A. Wakeham, D. Bouchard, W. C. Yeh, J. C. McGlade, P. S. Ohashi, and R. Hakem. 2003. Essential role for caspase 8 in T-cell homeostasis and T-cell-mediated immunity. Genes Dev 17:883-895.
Scaffidi, C., I. Schmitz, J. Zha, S. J. Korsmeyer, P. H. Krammer, and M. E. Peter. 1999. Differential modulation of apoptosis sensitivity in CD95 type I and type II cells. J Biol Chem 274:22532-22538.
Smith, K. G., A. Strasser, and D. L. Vaux. 1996. CrmA expression in T lymphocytes of transgenic mice inhibits CD95 (Fas/APO-1)-transduced apoptosis, but does not cause lymphadenopathy or autoimmune disease. EMBO J 15:5167-5176.
Strelow, A., K. Bernardo, S. Adam-Klages, T. Linke, K. Sandhoff, M. Kronke, and D. Adam. 2000. Overexpression of acid ceramidase protects from tumor necrosis factor-induced cell death. J Exp Med 192:601-612.
Tenev, T., K. Bianchi, M. Darding, M. Broemer, C. Langlais, F. Wallberg, A. Zachariou, J. Lopez, M. MacFarlane, K. Cain, and P. Meier. 2011. The Ripoptosome, a signaling platform that assembles in response to genotoxic stress and loss of IAPs. Mol Cell 43:432-448.
Upton, J. W., W. J. Kaiser, and E. S. Mocarski. 2010. Virus inhibition of RIP3-dependent necrosis. Cell Host Microbe 7:302-313.
Varfolomeev, E. E., M. Schuchmann, V. Luria, N. Chiannilkulchai, J. S. Beckmann, I. L. Mett, D. Rebrikov, V. M. Brodianski, O. C. Kemper, O. Kollet, T. Lapidot, D. Soffer, T. Sobe, K. B. Avraham, T. Goncharov, H. Holtmann, P. Lonai, and D. Wallach. 1998. Targeted disruption of the mouse Caspase 8 gene ablates cell death induction by the TNF receptors, Fas/Apo1, and DR3 and is lethal prenatally. Immunity 9:267-276.
Ventura, J. J., P. Cogswell, R. A. Flavell, A. S. Baldwin, Jr., and R. J. Davis. 2004. JNK potentiates TNF-stimulated necrosis by increasing the production of cytotoxic reactive oxygen species. Genes Dev 18:2905-2915.
Villena, J., M. Henriquez, V. Torres, F. Moraga, J. Diaz-Elizondo, C. Arredondo, M. Chiong, C. Olea-Azar, A. Stutzin, S. Lavandero, and A. F. Quest. 2008. Ceramide-induced formation of ROS and ATP depletion trigger necrosis in lymphoid cells. Free Radic Biol Med 44:1146-1160.
Wang, C. Y., M. W. Mayo, and A. S. Baldwin, Jr. 1996. TNF- and cancer therapy-induced apoptosis: potentiation by inhibition of NF-kappaB. Science 274:784-787.
Wang, L., F. Du, and X. Wang. 2008. TNF-alpha induces two distinct caspase-8 activation pathways. Cell 133:693-703.
Weinlich, R., C. P. Dillon, and D. R. Green. 2011. Ripped to death. Trends Cell Biol. in press:
Wertz, I. E., K. M. O'Rourke, H. Zhou, M. Eby, L. Aravind, S. Seshagiri, P. Wu, C. Wiesmann, R. Baker, D. L. Boone, A. Ma, E. V. Koonin, and V. M. Dixit. 2004. De-ubiquitination and ubiquitin ligase domains of A20 downregulate NF-kappaB signalling. Nature 430:694-699.
Yeh, W. C., A. Itie, A. J. Elia, M. Ng, H. B. Shu, A. Wakeham, C. Mirtsos, N. Suzuki, M. Bonnard, D. V. Goeddel, and T. W. Mak. 2000. Requirement for Casper (c-FLIP) in regulation of death receptor-induced apoptosis and embryonic development. Immunity 12:633-642.
Yeh, W. C., J. L. Pompa, M. E. McCurrach, H. B. Shu, A. J. Elia, A. Shahinian, M. Ng, A. Wakeham, W. Khoo, K. Mitchell, W. S. El-Deiry, S. W. Lowe, D. V. Goeddel, and T. W. Mak. 1998. FADD: essential for embryo development and signaling from some, but not all, inducers of apoptosis. Science 279:1954-1958.
Zhang, D. W., J. Shao, J. Lin, N. Zhang, B. J. Lu, S. C. Lin, M. Q. Dong, and J. Han. 2009. RIP3, an energy metabolism regulator that switches TNF-induced cell death from apoptosis to necrosis. Science 325:332-336.
Zhang, H., X. Zhou, T. McQuade, J. Li, F. K. Chan, and J. Zhang. 2011. Functional complementation between FADD and RIP1 in embryos and lymphocytes. Nature 471:373-376.
Zhang, J., D. Cado, A. Chen, N. H. Kabra, and A. Winoto. 1998. Fas-mediated apoptosis and activation-induced T-cell proliferation are defective in mice lacking FADD/Mort1. Nature 392:296-300.

### REFERENCE LIST 3

1a. Vandenabeele, P., Galluzzi, L., Vanden Berghe, T. & Kroemer, G. Molecular mechanisms of necroptosis: an ordered cellular explosion. Nat Rev Mol Cell Biol 11, 700-714, doi:10.1038/nrm2970 (2010).
2a. Vercammen, D., Vandenabeele, P., Beyaert, R., Declercq, W. & Fiers, W. Tumour necrosis factor-induced necrosis versus anti-Fas-induced apoptosis in L929 cells. Cytokine 9, 801-808, doi:S1043-4666(97)90252-0 [pii] 10.1006/cyto.1997.0252 (1997).
3a. Degterev, A. et al. Identification of RIP1 kinase as a specific cellular target of necrostatins. Nature chemical biology 4, 313-321, doi:nchembio.83 [pii] 10.1038/nchembio.83 (2008).
4a. Lin, Y. et al. Tumor necrosis factor-induced nonapoptotic cell death requires receptor-interacting protein-mediated cellular reactive oxygen species accumulation. The Journal of biological chemistry 279, 10822-10828, doi:10.1074/jbc.M313141200 (2004).
5a. Cho, Y. S. et al. Phosphorylation-driven assembly of the RIP1-RIP3 complex regulates programmed necrosis and virus-induced inflammation. Cell 137, 1112-1123, doi:S0092-8674(09)00642-4 [pii] 10.1016/j.cell.2009.05.037 (2009).
6a. Zhang, D. W. et al. RIP3, an energy metabolism regulator that switches TNF-induced cell death from apoptosis to necrosis. Science 325, 332-336, doi:1172308 [pii] 10.1126/science. 1172308 (2009).
7a. He, S. et al. Receptor interacting protein kinase-3 determines cellular necrotic response to TNF-alpha. Cell 137, 1100-1111, doi:S0092-8674(09)00578-9 [pii] 10.1016/j.cell.2009.05.021 (2009).
8a. Vanden Berghe, T. et al. Necroptosis, necrosis and secondary necrosis converge on similar cellular disintegration features. Cell Death Differ 17, 922-930, doi:10.1038/cdd.2009.184 (2010).
9a. Thapa, R. J. et al. Interferon-induced RIP1/RIP3-mediated necrosis requires PKR and is licensed by FADD and caspases. Proc Natl Acad Sci USA 110, E3109-3118, doi:10.1073/pnas.1301218110 (2013).
10a. He, S., Liang, Y., Shao, F. & Wang, X. Toll-like receptors activate programmed necrosis in macrophages through a receptor-interacting kinase-3-mediated pathway. Proc Natl Acad Sci USA 108, 20054-20059, doi:10.1073/pnas.1116302108 (2011).
11a. Upton, J. W., Kaiser, W. J. & Mocarski, E. S. DAI/ZBP1/DLM-1 complexes with RIP3 to mediate virus-induced programmed necrosis that is targeted by murine cytomegalovirus vIRA. Cell host & microbe 11, 290-297, doi:10.1016/j.chom.2012.01.016 (2012).
12a. Upton, J. W., Kaiser, W. J. & Mocarski, E. S. Virus inhibition of RIP3-dependent necrosis. Cell host & microbe 7, 302-313, doi:10.1016/j.chom.2010.03.006 (2010).
13a. Robinson, N. et al. Type I interferon induces necroptosis in macrophages during infection with Salmonella enterica serovar Typhimurium. Nat Immunol 13, 954-962, doi:10.1038/ni.2397 (2012).
14a. Duprez, L. et al. RIP kinase-dependent necrosis drives lethal systemic inflammatory response syndrome. Immunity 35, 908-918, doi:10.1016/j.immuni.2011.09.020 (2011).
15a. Kelliher, M. A. et al. The death domain kinase RIP mediates the TNF-induced NF-kappaB signal. Immunity 8, 297-303 (1998).
16a. Wang, L., Du, F. & Wang, X. TNF-alpha induces two distinct caspase-8 activation pathways. Cell 133, 693-703, doi:10.1016/j.cell.2008.03.036 (2008).
17a. Li, J. et al. The RIP1/RIP3 necrosome forms a functional amyloid signaling complex required for programmed necrosis. Cell 150, 339-350, doi:10.1016/j.cell.2012.06.019 (2012).
18a. Feoktistova, M. et al. cIAPs Block Ripoptosome Formation, a RIP1/Caspase-8 Containing Intracellular Cell Death Complex Differentially Regulated by cFLIP Isoforms. Molecular cell, doi:10.1016/j.molcel.2011.06.011 (2011).
19a. Tenev, T. et al. The Ripoptosome, a Signaling Platform that Assembles in Response to Genotoxic Stress and Loss of IAPB. Molecular cell, doi:10.1016/j.molcel.2011.06.006 (2011).
20a. Chen, W. et al. Diverse sequence determinants control human and mouse receptor interacting protein 3 (RIP3) and mixed lineage kinase domain-like (MLKL) interaction in necroptotic signaling. J Biol Chem 288, 16247-16261, doi:10.1074/jbc.M112.435545 (2013).
21a. Wu, J. et al. Mlkl knockout mice demonstrate the indispensable role of Mlkl in necroptosis. Cell Res 23, 994-1006, doi:10.1038/cr.2013.91 (2013).
22a. Sun, L. et al. Mixed lineage kinase domain-like protein mediates necrosis signaling downstream of RIP3 kinase. Cell 148, 213-227, doi:10.1016/j.cell.2011.11.031 (2012).
23a. Zhao, J. et al. Mixed lineage kinase domain-like is a key receptor interacting protein 3 downstream component of TNF-induced necrosis. Proc Natl Acad Sci USA 109, 5322-5327, doi:10.1073/pnas.1200012109 (2012).
24a. Geserick, P. et al. Cellular IAPB inhibit a cryptic CD95-induced cell death by limiting RIP1 kinase recruitment. J Cell Biol 187, 1037-1054, doi:jcb.200904158 [pii] 10.1083/jcb.200904158 (2009).
25a. Oberst, A. et al. Catalytic activity of the caspase-8-FLIP(L) complex inhibits RIPK3-dependent necrosis. Nature 471, 363-367, doi:10.1038/nature09852 (2011).
26a. Kaiser, W. J. et al. RIP3 mediates the embryonic lethality of caspase-8-deficient mice. Nature 471, 368-372, doi:10.1038/nature09857 (2011).
27a. Clackson, T. et al. Redesigning an FKBP-ligand interface to generate chemical dimerizers with novel specificity. Proc Natl Acad Sci USA 95, 10437-10442 (1998).
28a. Oberst, A. et al. Inducible dimerization and inducible cleavage reveal a requirement for both processes in caspase-8 activation. J Biol Chem 285, 16632-16642, doi:10.1074/jbc.M109.095083 (2010).
29a. Pop, C. et al. FLIP(L) induces caspase 8 activity in the absence of interdomain caspase 8 cleavage and alters substrate specificity. The Biochemical journal 433, 447-457, doi:10.1042/BJ20101738 (2011).
30a. Chang, D. W. & Yang, X. Activation of procaspases by FK506 binding protein-mediated oligomerization. Sci STKE 2003, PL1 (2003).
31a. Vanden Berghe, T. et al. Differential signaling to apoptotic and necrotic cell death by Fas-associated death domain protein FADD. J Biol Chem 279, 7925-7933, doi:10.1074/jbc.M307807200 M307807200 [pii] (2004).
32a. Dillon, C. P. et al. Survival function of the FADD-CASPASE-8-cFLIP(L) complex. Cell reports 1, 401-407, doi:10.1016/j.celrep.2012.03.010 (2012).
33a. Banaszynski, L. A., Chen, L. C., Maynard-Smith, L. A., Ooi, A. G. & Wandless, T. J. A rapid, reversible, and tunable method to regulate protein function in living cells using synthetic small molecules. Cell 126, 995-1004 (2006).
34a. Kaiser, W. J. et al. Toll-like Receptor 3-mediated Necrosis via TRIF, RIP3, and MLKL. J Biol Chem 288, 31268-31279, doi:10.1074/jbc.M113.462341 (2013).
35a. Murphy, J. M. et al. The pseudokinase MLKL mediates necroptosis via a molecular switch mechanism. Immunity 39, 443-453, doi:10.1016/j.immuni.2013.06.018 (2013).

1-5. (canceled)

6. A fusion protein comprising a truncated RIPK3 and at least two binding proteins.

7. The fusion protein of claim 6, wherein said truncated RIPK3 lacks a RHIM domain.

8. The fusion protein of claim 7, wherein the amino acid sequence of said RHIM domain is selected from the group consisting of SEQ ID NO:4 and SEQ ID NO: 5.

9. The fusion protein of claim 6, wherein said truncated RIPK3 lacks a C-terminal domain.

10. The fusion protein of claim 6, wherein one of said at least two binding proteins is selected from the group consisting of an Fv domain, an FK506 binding protein and an FRB binding protein.

11. (canceled)

12. An oligomer comprising at least one fusion protein comprising a truncated RIPK3 and at least two binding proteins.

13. The oligomer of claim 12, wherein said at least one fusion protein is attached at one said at least two binding proteins.

14. The oligomer of claim 12, wherein said at least one fusion protein is selected from the group consisting of at least three fusion proteins, at least four fusion proteins, at least five fusion proteins and at least six fusion proteins.

15-16. (canceled)

17. The oligomer of claim 12, wherein said oligomer is selected from the group consisting of a homodimer and a heterodimer.

18. (canceled)

19. The oligomer of claim 18, wherein said heterodimer comprises a first fusion protein comprising a truncated RIPK3 and a first binding protein, and a second fusion protein comprising RIPK1 and a second binding protein.

20. The oligomer of claim 19, wherein said first binding protein is a FK506 binding protein and said second binding protein is a FRB binding protein.

21. The oligomer of claim 19, wherein the amino acid sequence of said truncated RIPK3 is SEQ ID NO:2 and the amino acid sequence of said RIPK1 is SEQ ID NO: 3.

22. The oligomer of claim 19, wherein said first fusion protein is attached to said second fusion protein via said first and second binding proteins.

23-42. (canceled)

43. A method of inducing necroptosis, comprising:
a) providing:
i) a biological cell,
ii) a vector comprising a nucleic acid sequence encoding a fusion protein comprising a truncated RIPK3 and at least two binding proteins, and
iii) a dimerizing agent;
b) introducing said vector into said biological cell under conditions such that said fusion protein is expressed; and
c) contacting said biological cell with said dimerizing agent under conditions such that said expressed fusion proteins form a truncated RIPK3 oligomer;
d) inducing necroptosis in said biological cell with said truncated RIPK3 oligomer.

44. The method of claim 43, wherein said truncated RIPK3 lacks a RHIM domain.

45. The method of claim 43, wherein said truncated RIPK3 lacks a C-terminal domain.

46. The method of claim 43, wherein the amino acid sequence of said truncated RIPK3 is SEQ ID NO: 2.

47. The method of claim 44, wherein the amino acid sequence of said RHIM domain is selected from the group consisting of SEQ ID NO:4 and SEQ ID NO:5.

48. The method of claim 43, wherein said truncated RIPK3 oligomer a homodimer and a heterodimer.

49. (canceled)

50. The method of claim 43, wherein one said at least two binding proteins are selected from the group consisting of an Fv domain, an FK506 binding protein and an FRB binding protein.

51. The method of claim 43, wherein said dimerizing agent is rapamycin or a derivative thereof.

52. The method of claim 43, wherein said biological cell is a tumor cell.

53. The method of claim 52, wherein said tumor cell is derived from a patient diagnosed with cancer.

54. The method of claim 43, wherein said introducing comprises administering said vector into said patient.

55. The method of claim 54, wherein administering is selected from the group consisting of an intravenous injection, an intramuscular injection, a subcutaneous injection and an intratumoral injection.

56. The method of claim 43, wherein said contacting comprises administering said dimerizing agent to said patient.

57-78. (canceled)
